(19) United States
(12) Patent Application Publication
(10) Pub. No.: US 2026/0069193 A1
(43) Pub. Date: Mar. 12, 2026

(54) METHODS AND SYSTEMS FOR ASSESSING VISUAL PROCESSING SPEED AND ACCURACY IN VIRTUAL ENVIRONMENTS

(71) Applicant: Zenni Optical, Inc., Novato, CA (US)

(72) Inventors: Steven LEE, Barrington, IL (US); Julia ZHEN, Novato, CA (US); ChyrSong TING, Novato, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 18/827,588

(22) Filed: Sep. 6, 2024

(51) Int. Cl. A61B 5/00; G06T 7/20

(52) U.S. Cl. CPC A61B 5/4064; A61B 5/0059; A61B 5/4088; A61B 5/7445; G06T 7/20; A61B 2560/0487; G06T 2207/10016; G06T 2207/30041

(57) ABSTRACT

A vision test can be implemented based on a user's head orientation in a virtual environment. An electronic device, such as a head-mounted display, can execute a visual assessment application and can display a user interface to create a 3D virtual environment. While displaying a sequence of visual stimuli on the user interface, the electronic device can obtain a sequence of eye images of two eyes of a user associated with the electronic device, and the sequence of visual stimuli can correspond to a sequence of stimulus positions in the 3D virtual environment. The electronic device can determine a sequence of 3D gaze positions of the eyes in the 3D virtual environment based on the sequence of eye images. Further, the electronic device can determine a visual processing performance factor for the user based on the sequence of stimulus positions and the sequence of 3D gaze positions.

### TECHNICAL FIELD

The present disclosure relates to vision test technology. More specifically, methods, systems, devices, and non-statutory computer-readable storage media can be applied to enable vision testing associated with a user's visual processing speed and accuracy in an extended reality environment.

### BACKGROUND

Traditional methods for visual acuity assessment do not allow for dynamic adjustment of test parameters, leading to less accurate assessments, nor can they be implemented to test eyes and vision at home using household devices in a very environment locked manner.

### SUMMARY

The present disclosure relates to innovative methods and systems that can revolutionize vision care, making vision testing and other exams more accessible and affordable for patients. Additionally, it is contemplated that the principles and features of the present disclosure can be implemented in numerous other applications of display technology, including headsets, heads-up displays, and other microdisplays (e.g., microLED and microOLED) to address challenges and limitations inherent in such products and their uses.

Some implementations of the present disclosure are directed to a method for testing vision. The method is implemented at an electronic device having a head-mounted display (HMD), one or more processors, and memory. The method includes executing a visual assessment application (e.g., by displaying a user interface to create a 3D virtual environment). The method further includes while displaying a sequence of visual stimuli on the user interface, obtaining a sequence of eye images of two eyes of a user associated with the electronic device. The sequence of visual stimuli corresponds to a sequence of stimulus positions in the 3D virtual environment. The method further includes determining a sequence of 3D gaze positions of the eyes in the 3D virtual environment based on the sequence of eye images and determining a visual processing performance factor for the user based on the sequence of stimulus positions and the sequence of 3D gaze positions.

Some implementations of the present disclosure are directed to a method for testing vision. The method is implemented at an electronic device having a HMD, one or more processors, and memory. The method includes executing a visual assessment application (e.g., by displaying a user interface to create a 3D virtual environment. The method further includes: while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment; and applying a visual processing assessment model to receive the sequence of eye images and the sequence of stimulus positions as inputs and generate a visual processing performance factor for the user.

Some implementations of the present disclosure are directed to a method for testing vision. The method is implemented at an electronic device having a HMD, one or more processors, and memory. The method includes establishing a communication link between the electronic device and a controller held by a user associated with the electronic device; executing a user application configured to enable the vision test; displaying a VR user interface based on a driver license issuing requirement to create a 3D virtual environment, the VR user interface including a moving traffic scene on which one or more visual stimuli are displayed; and driving one or more actuators of a controller in synchronization with displaying the VR user interface.

Some implementations of the present disclosure are directed to a method for testing vision. The method is implemented at an electronic device having a HMD, one or more processors, and memory. The method includes establishing a communication link between the electronic device and a controller held by a user associated with the electronic device; executing a media play application to enable a 3D user interface; displaying media content on the 3D user interface; obtaining media metadata associated with the media content; generating a controller instruction based on the media metadata; and applying the controller instruction to drive one or more actuators of a controller in synchronization with the media content.

Some implementations of the present disclosure are directed to a method for testing vision. The method is implemented at an electronic device having a HMD, an infrared camera, one or more processors, and memory. The method includes executing a visual assessment application (e.g., by displaying a user interface to create a 3D virtual environment; displaying a body of text on the user interface for an extended duration of time; obtaining a sequence of eye images, each eye image including a respective infrared image of a region of interest (ROI) corresponding to at least one eye; based on the sequence of eye images, determining an eye endurance level of the at least one eye of a user associated with the electronic device.

Some implementations of the present disclosure are directed to a method for testing vision. The method is implemented at an electronic device having a HMD, an infrared camera, one or more processors, and memory. The method includes displaying a visual pattern on the user interface for an extended duration of time; obtaining a sequence of eye images from an eye-tracking camera, each eye diagram including a sclera area; and applying an eye endurance model to process the sequence of eye images and generate a model output including a dry eye indicator associated with a dry eye condition.

Some implementations of the present disclosure are directed to a method for testing vision. The method is implemented at an electronic device having a HMD, one or more processors, and memory. The method includes executing a visual assessment application (e.g., by displaying a user interface to create a 3D virtual environment; displaying a sequence of visual stimuli on the user interface, wherein the sequence of visual stimuli corresponds to a plurality of stimulus positions distributed in the 3D virtual environment; obtaining a sequence of eye images of two eyes of a user associated with the electronic device; determining a sequence of eye focal positions of the eyes in the sequence of eye images; and determining a convergence performance indicator for the two eyes of the user based on at least the sequence of eye focal positions.

Some implementations of the present disclosure are directed to a method for testing vision. The method is implemented at an electronic device having a HMD, one or more processors, and memory. The method includes executing a visual assessment application (e.g., by displaying a user interface to create a 3D virtual environment; displaying a sequence of visual stimuli on the user interface; obtaining a sequence of eye images of two eyes of a user associated with the electronic device; determining a sequence of eye focal positions of the two eyes in the sequence of eye images; and generating a map of convergence angles of the two eyes based on at least the sequence of eye focal positions.

Some implementations of the present disclosure are directed to a method for testing vision. The method is implemented at an electronic device having a HMD, one or more sensors, one or more processors, and memory. The method includes executing a visual assessment application (e.g., by displaying a user interface to create a 3D virtual environment; while displaying a sequence of visual hallucination patterns, obtaining a stream of sensor data from the one or more sensors; determining a plurality of user responses to the sequence of visual hallucination patterns based on the stream of sensor data; and determining a type and a severity level of a first visual hallucination condition of a user associated with the electronic device.

Some implementations of the present disclosure are directed to a method for testing vision. The method is implemented at an electronic device having a HMD, one or more sensors, one or more processors, and memory. The method includes executing a visual assessment application (e.g., by displaying a user interface to create a 3D virtual environment. The method further includes while displaying a sequence of visual hallucination patterns, obtaining a stream of sensor data from the one or more sensors; extracting a plurality of spontaneous response feature vectors from the sensor data; applying a hallucination diagnosis model to process at least the plurality of spontaneous response feature vectors and generate an output vector; and determining a type and a severity level of a first visual hallucination condition of a user associated with the electronic device.

Some implementations of the present disclosure are directed to a method for testing vision. The method is implemented at an electronic device having a HMD, one or more sensors, one or more processors, and memory. The method includes displaying a plurality of visual stimuli concurrently in a 3D virtual environment, each visual stimulus being displayed at a position in the 3D virtual environment according to a display scheme; obtaining a stream of sensor data measured by the one or more sensors; determining a plurality of sequential user responses to the plurality of visual stimuli based on the stream of sensor data; and based on the plurality of sequential user responses, determining an attention indicator indicating an attention capability of the user associated with the electronic device to different visual stimuli.

Some implementations of the present disclosure are directed to a method for testing vision. The method is implemented at an electronic device having a HMD, an infrared eye tracking camera, one or more processors, and memory. The method includes while displaying a plurality of visual stimuli concurrently in a 3D virtual environment, obtaining infrared video data recorded by the infrared eye tracking camera; determining a plurality of sequential user responses to the plurality of visual stimuli based on the infrared video data; and determining a severity level and a type of an attention deficiency condition for the user associated with the electronic device based on the plurality of sequential user responses.

Some implementations of the present disclosure are directed to a method for testing vision. The method is implemented at an electronic device having a HMD, one or more motion sensors, one or more processors, and memory. The method includes displaying a destination and a target path leading to the destination in a 3D virtual environment, the target path following at least one direction; rendering a request for a user associated with the electronic device to follow the target path to reach the destination; obtaining a stream of sensor data from the one or more motion sensors, the stream of sensor data being collected from the one or more motion sensors while the user moves along the target path; and based on the stream of sensor data, determining a directionality indicator of the user's visual system quantitatively representing a capability of the user's visual system following the at least one direction.

Some implementations of the present disclosure are directed to a method for testing vision. The method is implemented at an electronic device having a head-mounted display (HMD), one or more motion sensors, one or more processors, and memory. The method includes executing a sport training application for athlete training; displaying a destination and a target path leading to the destination in a 3D virtual environment; obtaining a stream of sensor data from the one or more motion sensors, the stream of sensor data being collected while the user moves along the target path; and based on the stream of sensor data, determining a directionality indicator of the user's visual system quantitatively representing a direction managing capability of the user's visual system.

In some embodiments, a user application can be implemented by an electronic device including a HMD and configured to create a customized extended reality (XR) environment for a user engaged on an XR information platform. Products may be rendered for the user in a three-dimension format in the XR environment, thereby facilitating eyewear selection and fitting. The XR can be an umbrella term encapsulating Augmented Reality (AR), Virtual Reality (VR), Mixed Reality (MR), and everything in between. In this application, any embodiments that apply a VR system can be implemented using an AR or MR system as well.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE FIGURES

Various features of illustrative embodiments of the inventions are described below with reference to the drawings. The illustrated embodiments are intended to illustrate, but not to limit, the inventions.

FIG. 1 is an example data processing environment having one or more servers communicatively coupled to one or more computer devices (e.g., a headset device), in accordance with some embodiments.

FIG. 2 is an environment in which a computer device (e.g., a headset device) is applied to facilitate visual assessment or eyewear fitting, in accordance with some embodiments.

FIG. 3 is a block diagram of a computer system (e.g., including a headset device) configured to implement vision assessment or eyewear fitting, in accordance with some embodiments.

FIG. 4 is a block diagram of a machine learning system for training and applying machine learning models (e.g., for glass making), in accordance with some embodiments.

FIG. 5A is a structural diagram of an example neural network applied to process input data in a machine learning model, and FIG. 5B is an example node in the neural network, in accordance with some embodiments.

FIG. 6A is an example tumbling E chart applied in a visual acuity test, and FIGS. 6B-6E are example patterns applied in an astigmatism test, a stereopsis test, a visual field test, and a color blindness test, in accordance with some embodiments.

FIG. 7 is another example visual pattern applied to test visual acuity and astigmatism, in accordance with some embodiments.

FIGS. 8A-8D include four diagrams of example graphical user interfaces rendered to determine a visual acuity score in a virtual environment created by a headset device, in accordance with some embodiments.

FIGS. 9A-9C include three diagrams of example graphical user interfaces rendered to determine a nearsighted or farsighted power in a virtual environment created by a headset device, in accordance with some embodiments.

FIGS. 10A-10F include six diagrams of example graphical user interfaces rendered to determine eye stigmatism in a virtual environment created by a headset device, in accordance with some embodiments.

FIG. 11 is a flow diagram of an example vision test process for determining visual processing speed and accuracy of a user's visual system, in accordance with some embodiments.

FIGS. 12A and 12B are flow diagrams of two example vision test processes for determining a visual processing performance factor of eyes of a user, in accordance with some embodiments, respectively.

FIG. 13 is a diagram of an example gaze path on which a gaze of a user's eyes approaches a visual stimulus, in accordance with some embodiments.

FIG. 14 is a flow diagram of an example process for actuating controllers in synchronization with a vision test, in accordance with some embodiments.

FIG. 15 is a flow diagram of an example vision test process for facilitating a vision test with a controller in a 3D virtual environment, in accordance with some embodiments.

FIG. 16 is an example traffic scene enabled in a virtual environment for a vision test facilitated by the controller 390, in accordance with some embodiments.

FIG. 17 is a flow diagram of an example vision test process for assessing visual endurance of a user's visual system, in accordance with some embodiments.

FIG. 18A is a flow diagram of an example vision test process for assessing an eye endurance level in a 3D virtual environment, in accordance with some embodiments.

FIG. 18B is a flow diagram of an example vision test process for assessing a dry eye condition in a 3D virtual environment, in accordance with some embodiments.

FIG. 19 is a flow diagram of an example vision test process for assessing convergence performance of a user's visual system, in accordance with some embodiments.

FIG. 20 is a diagram illustrating a convergence error of a user's eyes, in accordance with some embodiments.

FIG. 21 is a flow diagram of an example vision test process for assessing convergence performance of a user's eyes in a 3D virtual environment, in accordance with some embodiments.

FIG. 22 is a flow diagram of an example vision test process for assessing a hallucination condition of a user's visual system, in accordance with some embodiments.

FIG. 23 is a flow diagram of an example vision test process for assessing a hallucination condition of a user's visual system in a 3D virtual environment, in accordance with some embodiments.

FIG. 24 is a flow diagram of an example vision test process for determining visual processing speed and accuracy of a user's visual system, in accordance with some embodiments.

FIG. 25A is a flow diagram of an example vision test process for assessing a user's visual attention in a 3D virtual environment, in accordance with some embodiments.

FIG. 25B is a flow diagram of another example vision test process for assessing a user's visual attention in a 3D virtual environment, in accordance with some embodiments.

FIG. 26 is a flow diagram of an example vision test process for assessing spatial awareness and balance associated with a user's visual system, in accordance with some embodiments.

FIG. 27 is a diagram of a 3D visual environment including a target path 2702, in accordance with some embodiments.

FIG. 28 is a flow diagram of an example vision test process for assessing spatial awareness of a user's visual system in a 3D virtual environment, in accordance with some embodiments.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Moreover, various aspects of the present disclosure can be implemented in combination with aspects of other virtual-reality technology developed by the present applicant, for example, in copending U.S. Patent App. Nos. 63/560,623 (137034-5002), filed on Mar. 1, 2024, 63/569,095 (137034-5005), filed on Mar. 23, 2024, 63/642,571 (137034-5007), filed on May 3, 2024, 63/642,583 (137034-5009), filed on May 3, 2024, 63/642,593 (137034-5010), filed on May 3, 2024, 63/642,604 (137034-5011), filed on May 3, 2024, 63/644,457 (137034-5012), filed on May 8, 2024, Ser. No. 18/759,641 (137034-5018), filed on Jun. 28, 2024, Ser. No. 18/791,203 (137034-5036), filed on Jul. 31, 2024, and ZEN-8014-US/137034-5050, filed Sep. 6, 2024, the entireties of each of which is incorporated herein by reference. Aspects of these copending cases can be implemented in combination with some embodiments disclosed herein, whether in addition to features thereof or as an alternative to a particular feature of an embodiment disclosed herein.

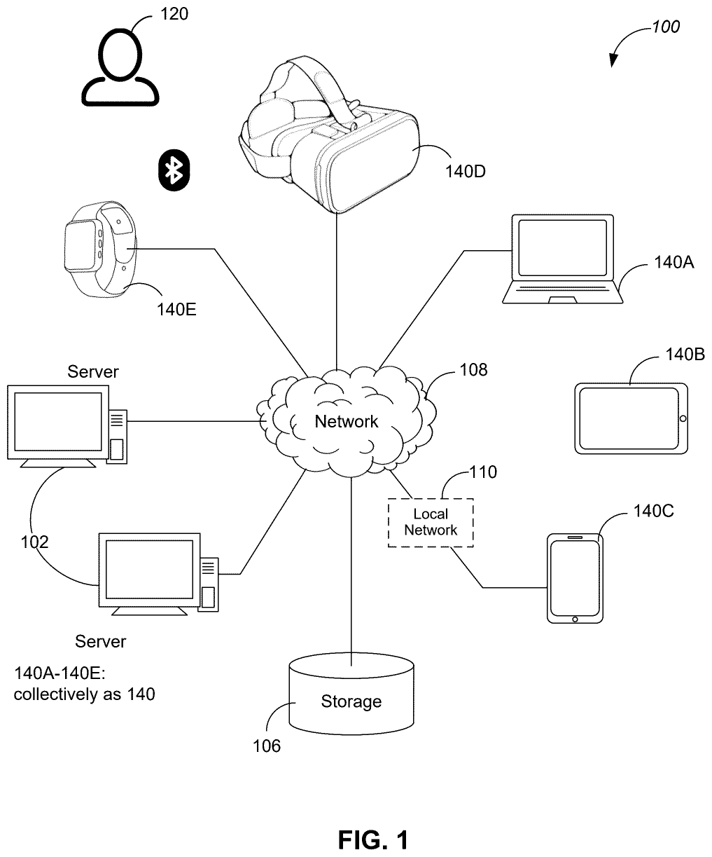

Referring now to the figures, FIG. 1 is an example data processing environment 100 having one or more servers 102 communicatively coupled to one or more computer devices 140 (e.g., a headset device 140D), in accordance with some embodiments. The one or more computer devices 140 are electronic devices having computational capabilities, and may be, for example, desktop computers 140A, tablet computers 140B, mobile phones 140C, or intelligent, multi-sensing, network-connected home devices (e.g., a depth camera, a visible light camera).

In some implementations, the one or more computer devices 140 can include a headset device 140D configured to render extended reality content. In some implementations, the one or more computer devices 140 can include a wireless wearable device 140E (e.g., a smart watch, a fitness band) configured to track health data (e.g., heart rate, quality of sleep) and activity data (e.g., steps walked, stairs climbed) of a user wearing the device 140E. Each computer device 140 can collect data or user inputs, executes user applications, and present outputs on its user interface. The collected data or user inputs can be processed locally at the computer device 140 and/or remotely by the server(s) 102. The one or more servers 102 can provide system data (e.g., boot files, operating system images, and user applications) to the computer devices 140, and in some embodiments, processes the data and user inputs received from the computer device(s) 140 when the user applications are executed on the computer devices 140. In some embodiments, the data processing environment 100 can further include a storage 106 for storing data related to the servers 102, computer devices 140, and applications executed on the computer devices 140. For example, storage 106 may store video content, static visual content, and/or audio data.

The one or more servers 102 can enable real time data communication with the computer devices 140 that can be remote from each other or from the one or more servers 102. Further, in some embodiments, the one or more servers 102 can implement data processing tasks that are not completed locally by the computer devices 140. For example, the computer devices 140 can include a game console (e.g., the headset device 140D) that executes an interactive online gaming application (e.g., for visual assessment or eyewear fitting). The game console receives a user instruction and sends it to a server 102 with user data. The server 102 generates a stream of video data based on the user instruction and user data, and provides the stream of video data for display on the game console and other computer devices that can be engaged in the same session with the game console.

The one or more servers 102, one or more computer devices 140, and storage 106 can be communicatively coupled to each other via one or more communication networks 108, which are the medium used to provide communications links between these devices and computers connected together within the data processing environment 100. The one or more communication networks 108 may include connections, such as wire, wireless communication links, or fiber optic cables. Examples of the one or more communication networks 108 include local area networks (LAN), wide area networks (WAN) such as the Internet, or a combination thereof. The one or more communication networks 108 are, optionally, implemented using any known network protocol includes various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocol. A connection to the one or more communication networks 108 may be established either directly (e.g., using 1G/4G connectivity to a wireless carrier), or through a network interface 110 (e.g., using a router, switch, gateway, hub, or an intelligent, dedicated whole-home control node), or through any combination thereof. As such, the one or more communication networks 108 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the headset device 140D can be communicatively coupled to a data processing environment 100. The headset device 140D includes one or more cameras (e.g., a visible light camera, a depth camera), a microphone, a speaker, one or more inertial sensors (e.g., gyroscope, accelerometer), and a display. In some embodiments, the camera may capture hand gestures of a user wearing the headset device 140D. In some embodiments, the microphone records ambient sound includes user's voice commands.

In some embodiments, the headset device 140D may be communicatively coupled to one or more servers 102 and enables a centralized vision test management platform with the one or more servers 102. This vision test management platform may aggregate data (e.g., visual stimuli 338, sensor data 342, vision test results 344) from a plurality of user accounts associated with a plurality of users, analyze the aggregated data, and track vision health trends for individual users or user groups. In some embodiments, data may be communicated between a headset device 140D and a server 102 in an encrypted format. In some embodiments, the vision test management platform is coupled to a global health database storing epidemiological data. The vision test management platform can be configured to cross-reference the data collected from its user accounts with the epidemiological data to identify an emerging pattern and a public health concern. For example, a teenager's vision data may be collected and analyzed during an extended duration of time (e.g., 10 years) to identify an individual vision development trend and was cross-referenced with an average vision development trend extracted from the global health database. A doctor can rely on a cross-referencing result to determine whether the individual vision development trend is normal or whether the teenager's eyesight drops faster than average teenagers. As such, various embodiments of the vision test management platform may integrate biometric data and global health analytics and provides a secure, personalized, and interactive environment for vision testing, which can improve precision and user experience of vision assessments and contributes to broader public health monitoring and research initiatives.

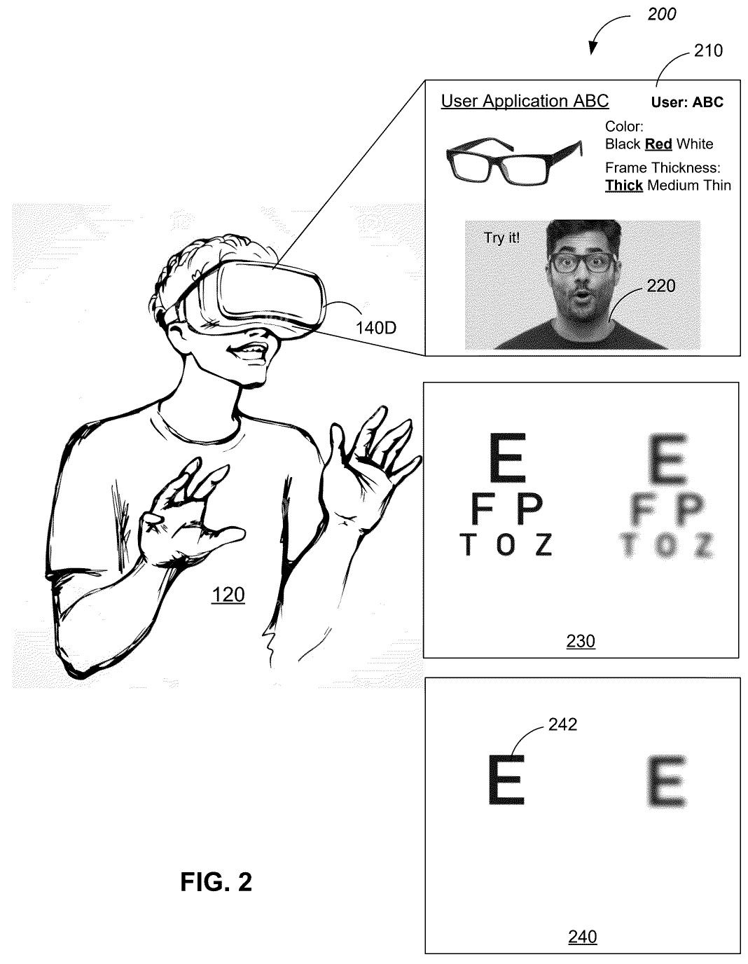

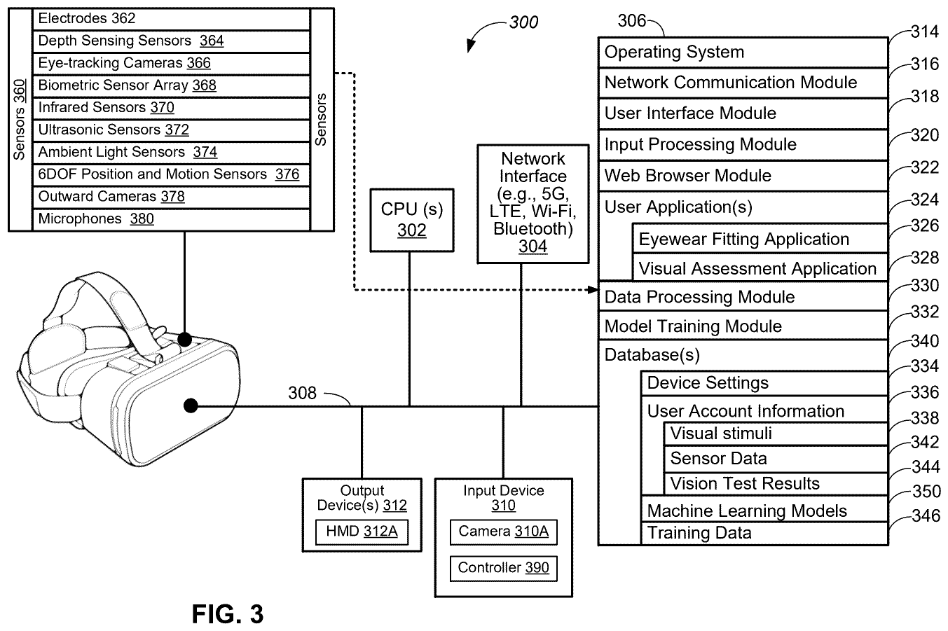

FIG. 2 is an environment 200 in which a computer device 140 (e.g., a headset device 140D) is applied to facilitate visual assessment or eyewear fitting, in accordance with some embodiments. The XR headset device 140D may be communicatively coupled within the data processing environment 100. The XR headset device 140D may include one or more cameras (e.g., a visible light camera, a depth camera), a microphone, a speaker, one or more inertial sensors (e.g., gyroscope, accelerometer), and a display. In some embodiments, the camera may capture hand gestures of a user wearing the XR headset device 140D. In some embodiments, the microphone may record ambient sound includes user's voice commands. The XR headset device 140D may execute a client-side eyewear fitting application 326 or a client-side visual assessment application 328 (FIG. 3) via a user account associated with a user 120 (e.g., an optometrist user, an optician user, a patient user). In some implementations, a computer device 140 (e.g., a mobile phone 140C) distinct from the XR headset device 140D can be used to implement the client-side eyewear fitting application 326 or visual assessment application 328 (FIG. 3).

In some embodiments, a first user interface 210 can be displayed on a computer device 140 (e.g., the headset device 140D) associated with the user 120. In some embodiments, an eyewear can be tried on or displayed as being worn by a 2D or 3D image 220 of the user 120. The server 102 or computer device 140 may receive from the first user interface 210, a user feedback message indicating an issue, requesting further improvement, or confirming a fit. In some embodiments, a second user interface 230 can be displayed on a computer device 140 associated with the user 120. The second user interface 230 may include a plurality of optotypes (e.g., six optotypes E, F, P, T, O, and Z) having different sizes. In some embodiments, a third user interface 240 can be displayed on a computer device 140 associated with the user 120. The second user interface 230 can display a temporal sequence of optotypes having respective sizes. Each optotype of a corresponding size can be displayed at one time.

FIG. 3 is a block diagram of a computer system 300 (e.g., including a headset device 140D, a server, or a combination thereof) configured to implement vision assessment or eyewear fitting, in accordance with some embodiments. The computer system 300 can include one or more processing units (CPUs) 302, one or more network interfaces 304, memory 306, and one or more communication buses 308 for interconnecting these components (sometimes called a chipset). The computer system 300 may include one or more input devices 310 that facilitate user input, such as a keyboard, a mouse, a voice-command input unit or microphone, a touch screen display, a touch-sensitive input pad, a gesture capturing camera, a controller 390, or other input buttons or controls. Furthermore, in some embodiments, the computer device 140 of the computer system 300 may use a microphone for voice recognition or an eye tracking camera 366 for tracking eyeball movement. In some implementations, the computer device 140 may include one or more optical cameras (e.g., an RGB camera), scanners, or photo sensor units for capturing images. The computer system 300 may also include one or more output devices 312 that enable presentation of user interfaces 210 and media content. The one or more output devices 312 may include one or more speakers and/or one or more visual displays.

The computer system 300 may include one or more sensors 360, which further may include one or more of: a plurality of electrodes 362, one or more depth sensing sensors 364, one or more eye tracking cameras 366, a biometric sensor array 368, one or more infrared sensors 370, one or more ultrasonic sensors 372, one or more ambient sensors 374, one or more motion sensors (e.g., six degree of freedom (6DOF) position and motion sensors 376), one or more outward camera 378, and one or more microphones 380. It is noted that the one or more sensors 360 can also be included in the input device 310 and used to collect data to the computer system 300.

Memory 306 may include high-speed random-access memory, such as DRAM, SRAM, DDR RAM, or other random-access solid state memory devices; and, optionally, may include non-volatile memory, such as one or more magnetic disk storage devices, one or more optical disk storage devices, one or more flash memory devices, or one or more other non-volatile solid state storage devices. Memory 306, optionally, may include one or more storage devices remotely located from one or more processing units 302. Memory 306, or alternatively the non-volatile memory within memory 306, may include a non-transitory computer readable storage medium. In some implementations, memory 306, or the non-transitory computer readable storage medium of memory 306, may store the following programs, modules, and data structures, or a subset or superset thereof:
Operating system 314 including procedures for handling various basic system services and for performing hardware dependent tasks;
Network communication module 316 for connecting each server 102 or computer device 140 to other devices (e.g., server 102, computer device 140, or storage 106) via one or more network interfaces 304 (wired or wireless) and one or more communication networks 108, such as the Internet, other wide area networks, local area networks, metropolitan area networks, and so on;
User interface module 318 for enabling presentation of information (e.g., a graphical user interface for application(s) 324, widgets, websites and web pages thereof, and/or games, audio and/or video content, text, etc.) at each computer device 140 via one or more output devices 312 (e.g., displays, speakers, etc.);
Input processing module 320 for detecting one or more user inputs or interactions from one of the one or more input devices 310 and interpreting the detected input or interaction;
Web browser module 322 for navigating, requesting (e.g., via HTTP), and displaying websites and web pages thereof may include a web interface for logging into a user account associated with a computer device 140 or another electronic device, controlling the computer device if associated with the user account, and editing and reviewing settings and data that are associated with the user account;
One or more user applications 324 for execution by the computer system 300 (e.g., games, social network applications, smart home applications, extended reality application, and/or other web or non-web-based applications for controlling another electronic device and reviewing data captured by such devices), where in some embodiments, an eyewear fitting application 326 can be executed to implement eyewear fitting, and has a plurality of user accounts associated with a plurality of users 120 (e.g., technician users and eyewear users), and in some embodiments, a visual assessment application 328 can be executed to evaluate eyesight of a patient user, and has a plurality of user accounts associated with a plurality of users 120 (e.g., an optometrist user, a patient user);
Data processing module 330 for processing data associated with the user applications 324, e.g., using machine learning models 350;
Model training module 332 for obtaining training data 346 and training machine learning models 350; and
One or more databases 340 for storing at least data including one or more of:
   Device settings 334 including common device settings (e.g., service tier, device model, storage capacity, processing capabilities, communication capabilities, etc.) of the computer system 300;
   User account information 336 for the one or more user applications 324, e.g., user names, security questions, account history data, user preferences, and predefined account settings, where in some embodiments, the user account information 336 may include facial measurements and one or more virtual fitting parameters associated with associated with a user account of an eye fitting application 326, and in some embodiments, the user account information 336 may include visual stimuli 338, sensor data 342, and vision test results 344 associated with a user account of a visual assessment application 328; and
   Machine learning models 350 including parameters (e.g., weights, biases) used to implement vision test or select eyewear for eyewear users.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in some embodiments. In some embodiments, memory 306, optionally, stores a subset of the modules and data structures identified above. Furthermore, memory 306, optionally, stores additional modules and data structures not described above.

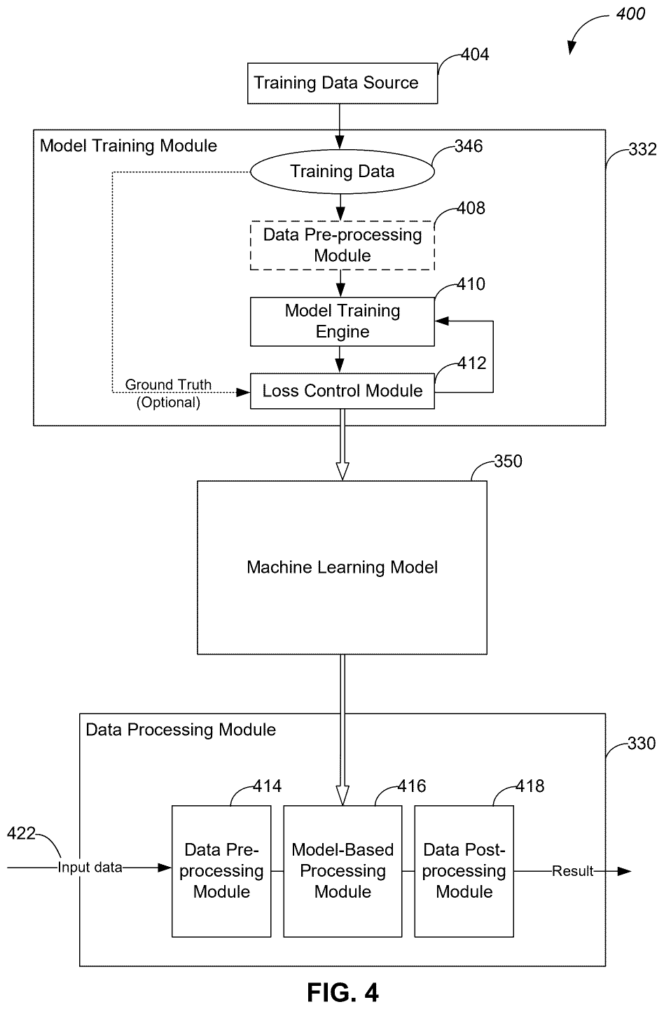

FIG. 4 is a block diagram of a machine learning system 400 for training and applying machine learning models 350 (e.g., for glass making), in accordance with some embodiments. The machine learning system 400 may include a model training module 332 establishing one or more machine learning models 350 and a data processing module 330 for processing input data 422 using the machine learning model 350. In some embodiments, both the model training module 332 and the data processing module 330 may be located within a computer device 140 (e.g., a VR headset), while a training data source 404 provides training data 346 to the computer device 140. In some embodiments, the training data source 404 may include the data obtained from the computer device 140 itself, from a server 102, from storage 106, or from another electronic device or computer device 140. Alternatively, in some embodiments, the model training module 332 may be located at a server 102, and the data processing module 330 may be located in a computer device 140. The server 102 can train the machine learning model 350 and provide the trained models 350 to the computer device 140 to process real time input data 422 detected by the computer device 140. In some embodiments, the training data 346 provided by the training data source 404 may include a standard dataset widely used to train machine learning models 350. The input data 422 further may include sensor data. Further, in some embodiments, a subset of the training data 346 may be modified to augment the training data 346. The subset of modified training data may be used in place of or jointly with the subset of training data 346 to train the machine learning models 350.

In some embodiments, the model training module 332 may include a model training engine 410, and a loss control module 412. Each machine learning model 350 may be trained by the model training engine 410 to process corresponding input data 422 and implement a respective task. Specifically, the model training engine 410 may receive the training data 346 corresponding to a machine learning model 350 to be trained and process the training data to build the machine learning model 350. In some embodiments, during this process, the loss control module 412 can monitor a loss function comparing the output associated with the respective training data item to a ground truth of the respective training data item. In these embodiments, the model training engine 410 may modify the machine learning models 350 to reduce the loss, until the loss function satisfies a loss criteria (e.g., a comparison result of the loss function is minimized or reduced below a loss threshold). The machine learning models 350 may thereby be trained and provided to the data processing module 330 of a computer device 140 to process real time input data 422 from the computer device 140.

In some embodiments, the model training module 402 may further include a data pre-processing module 408 configured to pre-process the training data 346 before the training data 346 is used by the model training engine 410 to train a machine learning model 350. For example, an image pre-processing module 408 is configured to format patients' eye images in the training data 346 into a predefined image format. For example, the preprocessing module 408 may normalize the images to a fixed size, resolution, or contrast level. In another example, an image pre-processing module 408 extracts a region of interest (ROI) corresponding to an eye area.

In some embodiments, the model training module 332 can use supervised learning in which the training data 346 may be labelled and include a desired output for each training data item (also called the ground truth, in some embodiments). In some embodiments, the desirable output may be labelled manually by people or automatically by the model training module 332 before training. In some embodiments, the model training module 332 may use unsupervised learning in which the training data 346 is not labelled. The model training module 332 is configured to identify previously undetected patterns in the training data 346 without pre-existing labels and with little or no human supervision. Additionally, in some embodiments, the model training module 332 may use partially supervised learning in which the training data is partially labelled.

In some embodiments, the data processing module 330 may include a data pre-processing module 414, a model-based processing module 416, and a data post-processing module 418. The data pre-processing modules 414 may pre-process input data 422 based on the type of the input data 422. In some embodiments, functions of the data pre-processing modules 414 are consistent with those of the pre-processing module 408. The data pre-processing modules 414 can convert the input data 422 into a predefined data format that is suitable for the inputs of the model-based processing module 416. The model-based processing module 416 may apply the trained machine learning model 350 provided by the model training module 332 to process the pre-processed input data 422. In some embodiments, the model-based processing module 416 can also monitor an error indicator to determine whether the input data 422 has been properly processed in the machine learning model 350. In some embodiments, the processed input data may be further processed by the data post-processing module 418 to create a preferred format or to provide additional information that can be derived from the processed input data. The data processing module 330 may use the processed input data to make eyewear glasses for a patient user.

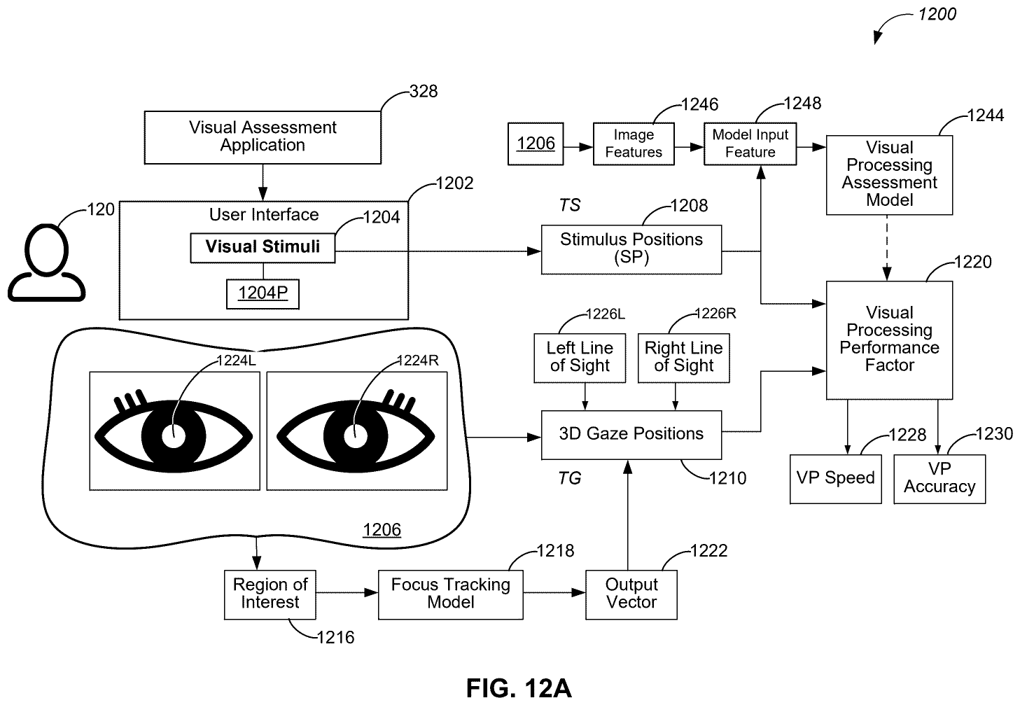

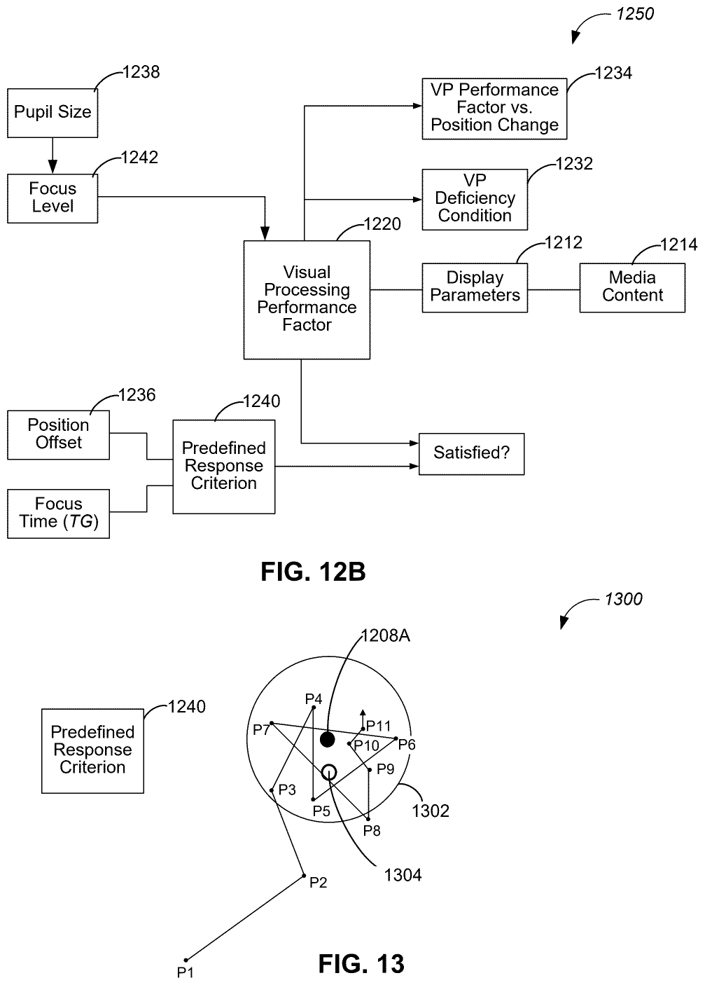

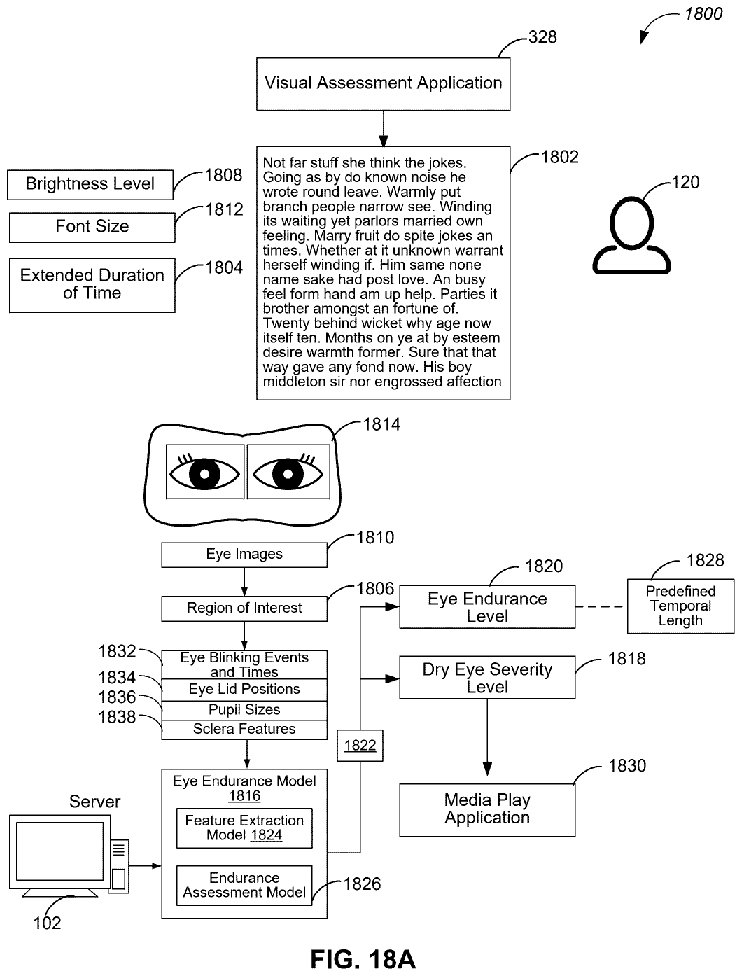

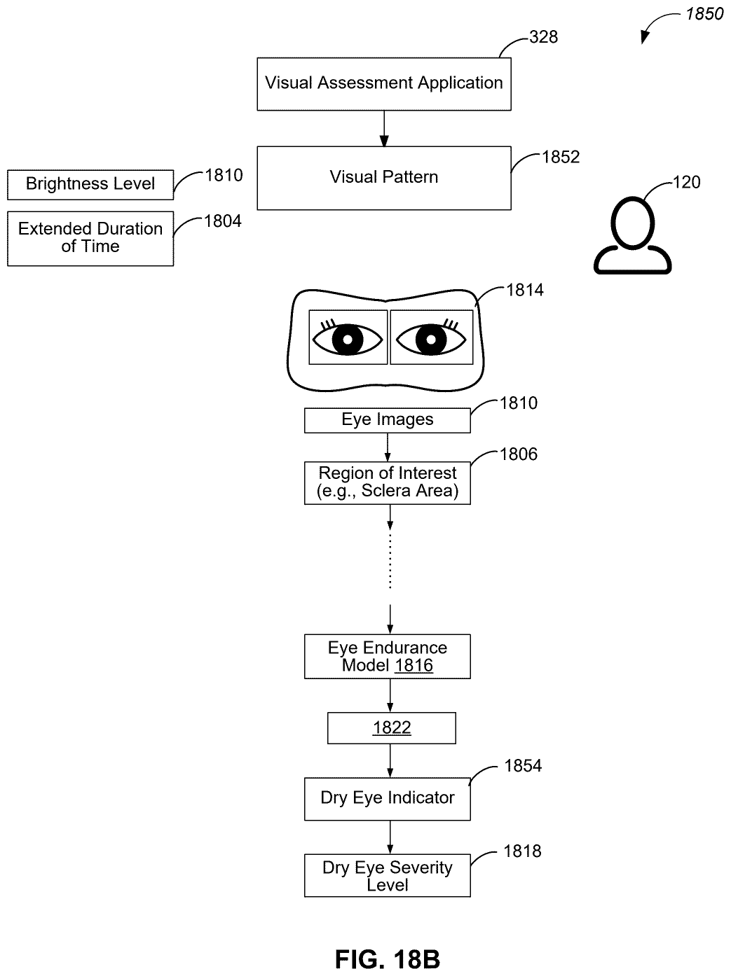

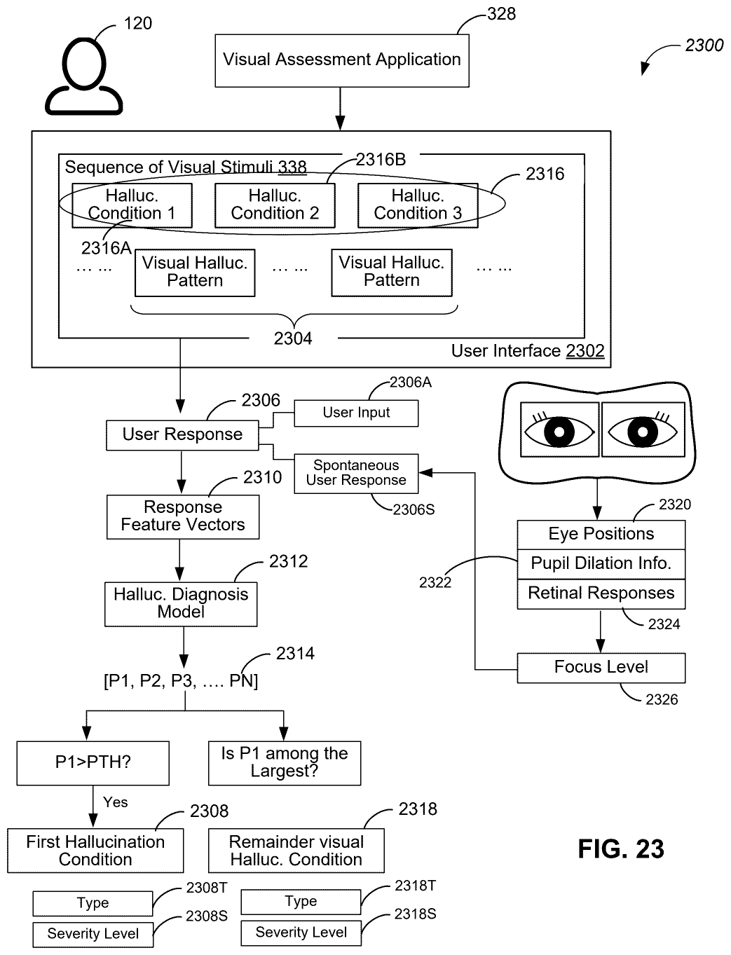

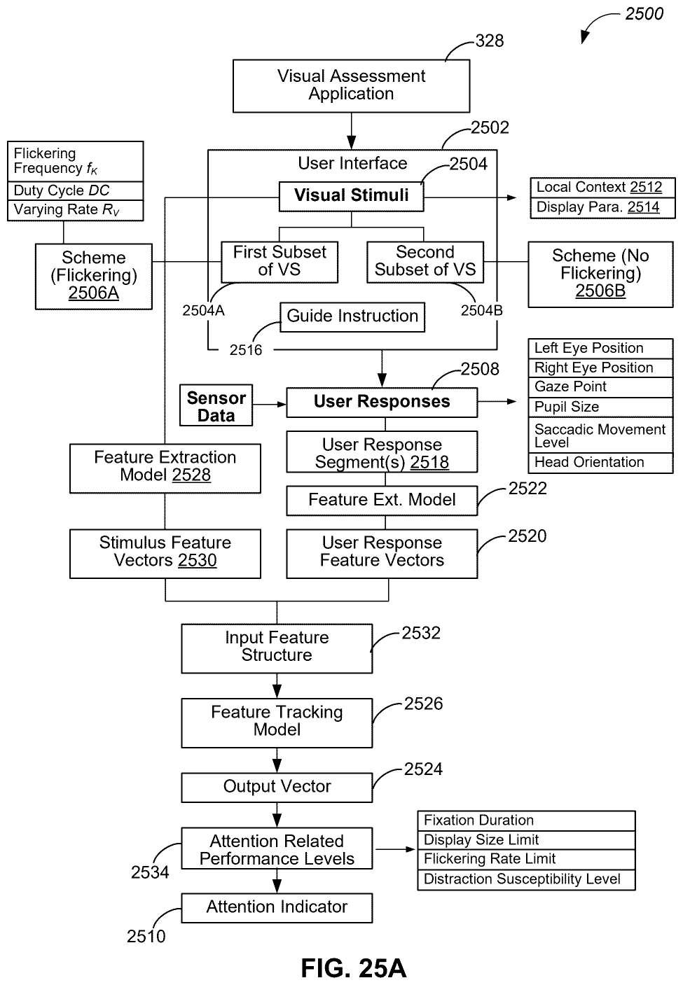

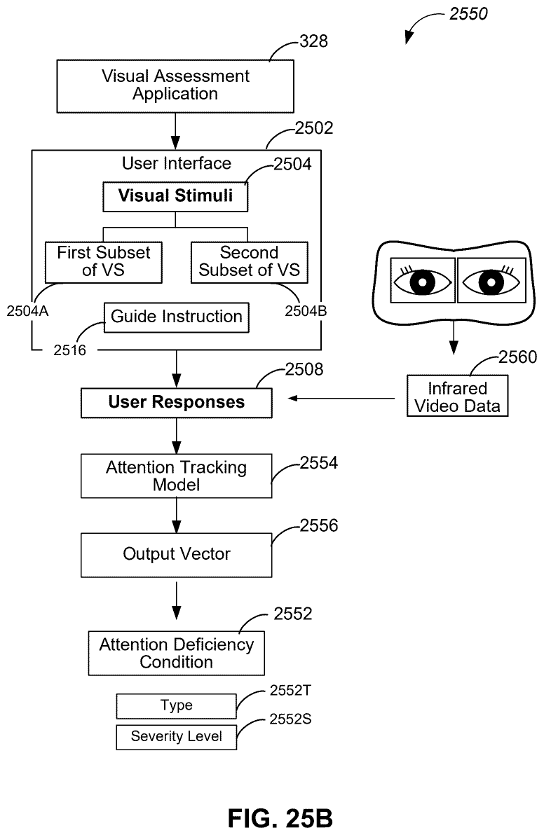

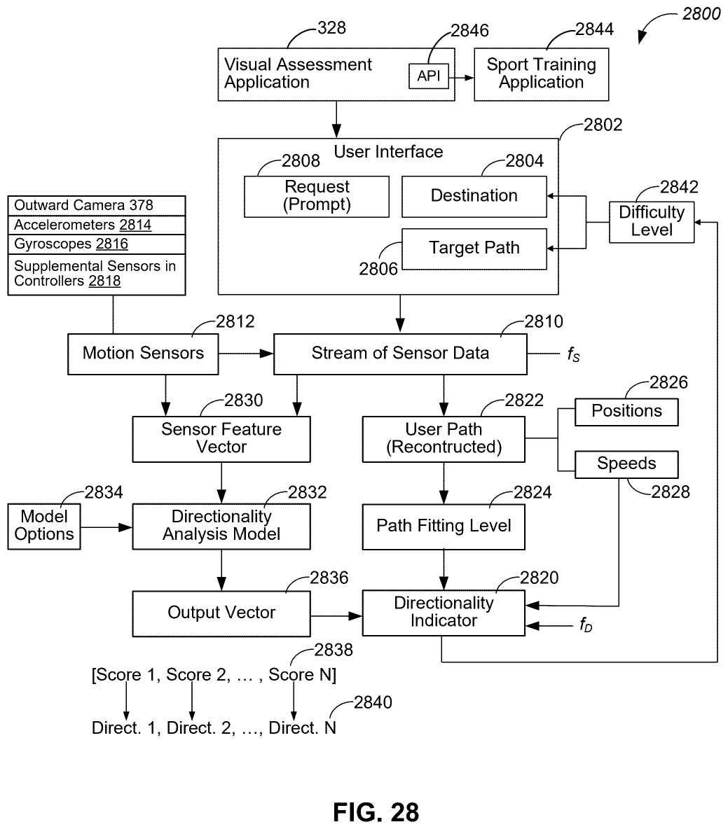

Examples of the machine learning model 350 include, but are not limited to, a focus tracking model 1218 (FIG. 12), a visual processing assessment model 1244 (FIG. 12), an eye endurance model 1816 (FIG. 18A), a feature extraction model 1824 (FIG. 18A), an endurance assessment model 1826 (FIG. 18A), a convergence angle model, a hallucination diagnosis model 2312 (FIG. 23), an attention tracking model 2554 (FIG. 25B), a feature tracking model 2526 (FIG. 25A), and a directionality analysis model 2832 (FIG. 28).

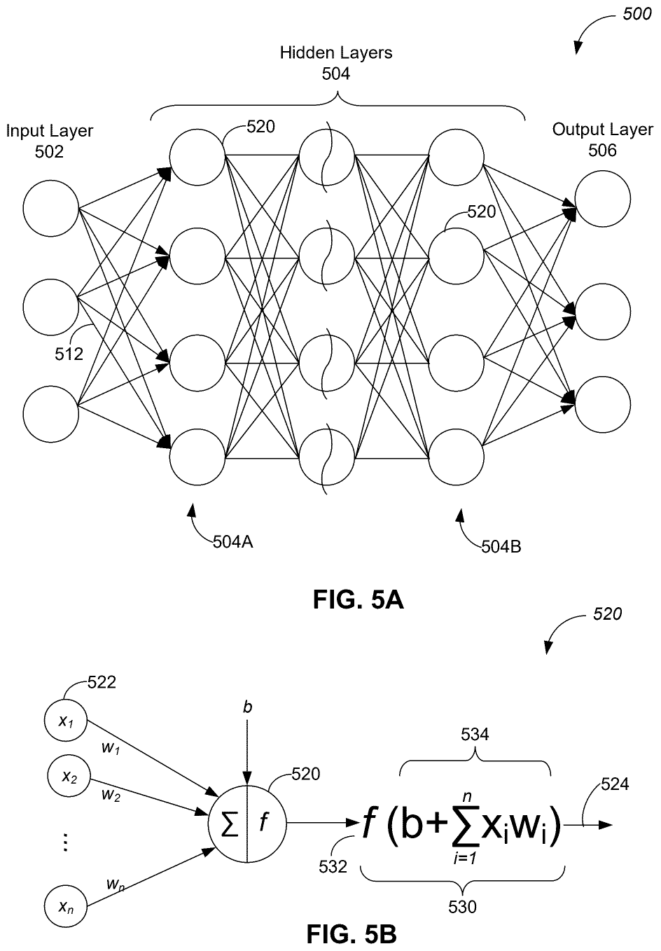

FIG. 5A is a structural diagram of an example neural network 500 applied to process input data in a machine learning model 350, in accordance with some embodiments. Further, FIG. 5B is an example node 520 in the neural network 500, in accordance with some embodiments. It should be noted that this description is used as an example only, and other types or configurations may be used to implement the embodiments described herein. The machine learning model 350 may be established based on the neural network 500. A corresponding model-based processing module 416 may apply the machine learning model 350 including the neural network 500 to process input data 422 that has been converted to a predefined data format. The neural network 500 may include a collection of nodes 520 that may be connected by links 512. Each node 520 may receive one or more node inputs 522 and applies a propagation function 530 to generate a node output 524 from the one or more node inputs. As the node output 524 is provided via one or more links 512 to one or more other nodes 520, a weight w associated with each link 512 may be applied to the node output 524. Likewise, the one or more node inputs 522 may be combined based on corresponding weights w₁, w₂, w₃, and w₄ according to the propagation function 530. In an example, the propagation function 530 is computed by applying a non-linear activation function 532 to a linear weighted combination 534 of the one or more node inputs 522.

The collection of nodes 520 may be organized into layers in the neural network 500. In general, the layers may include an input layer 502 for receiving inputs, an output layer 506 for providing outputs, and one or more hidden layers 504 (e.g., layers 504A and 504B) between the input layer 502 and the output layer 506. A deep neural network has more than one hidden layer 504 between the input layer 502 and the output layer 506. In the neural network 500, each layer may only be connected with its immediately preceding and/or immediately following layer. In some embodiments, a layer may be a fully connected layer because each node in the layer is connected to every node in its immediately following layer. In some embodiments, a hidden layer 504 may include two or more nodes that may be connected to the same node in its immediately following layer for down sampling or pooling the two or more nodes. In particular, max pooling may use a maximum value of the two or more nodes in the layer for generating the node of the immediately following layer.

In some embodiments, a convolutional neural network (CNN) may be applied in a machine learning model 350 to process input data. The CNN employs convolution operations and belongs to a class of deep neural networks. The hidden layers 504 of the CNN include convolutional layers. Each node in a convolutional layer may receive inputs from a receptive area associated with a previous layer (e.g., nine nodes). Each convolution layer may use a kernel to combine pixels in a respective area to generate outputs. For example, the kernel may be to a 3×3 matrix including weights applied to combine the pixels in the respective area surrounding each pixel. Video or image data can be pre-processed to a predefined video/image format corresponding to the inputs of the CNN. In some embodiments, the pre-processed video or image data may be abstracted by the CNN layers to form a respective feature map. In this way, video and image data can be processed by the CNN for video and image recognition or object detection.

In some embodiments, a recurrent neural network (RNN) is applied in the machine learning model 350 to process input data 422. Nodes in successive layers of the RNN follow a temporal sequence, such that the RNN exhibits a temporal dynamic behavior. In an example, each node 520 of the RNN has a time-varying real-valued activation. It is noted that in some embodiments, two or more types of input data may be processed by the data processing module 330, and two or more types of neural networks (e.g., both a CNN and an RNN) may be applied in the same machine learning model 350 to process the input data jointly.

The training process is a process for calibrating all of the weights w₁ for each layer of the neural network 500 using training data 346 that is provided in the input layer 502. The training process typically may include two steps, forward propagation and backward propagation, which may be repeated multiple times until a predefined convergence condition is satisfied. In the forward propagation, the set of weights for different layers may be applied to the input data and intermediate results from the previous layers. In the backward propagation, a margin of error of the output (e.g., a loss function) is measured (e.g., by a loss control module 412), and the weights may be adjusted accordingly to decrease the error. The activation function 532 can be linear, rectified linear, sigmoidal, hyperbolic tangent, or other types. In some embodiments, a network bias term b may be added to the sum of the weighted outputs 534 from the previous layer before the activation function 532 is applied. The network bias b may provide a perturbation that helps the neural network 500 avoid over fitting the training data. In some embodiments, the result of the training may include a network bias parameter b for each layer.

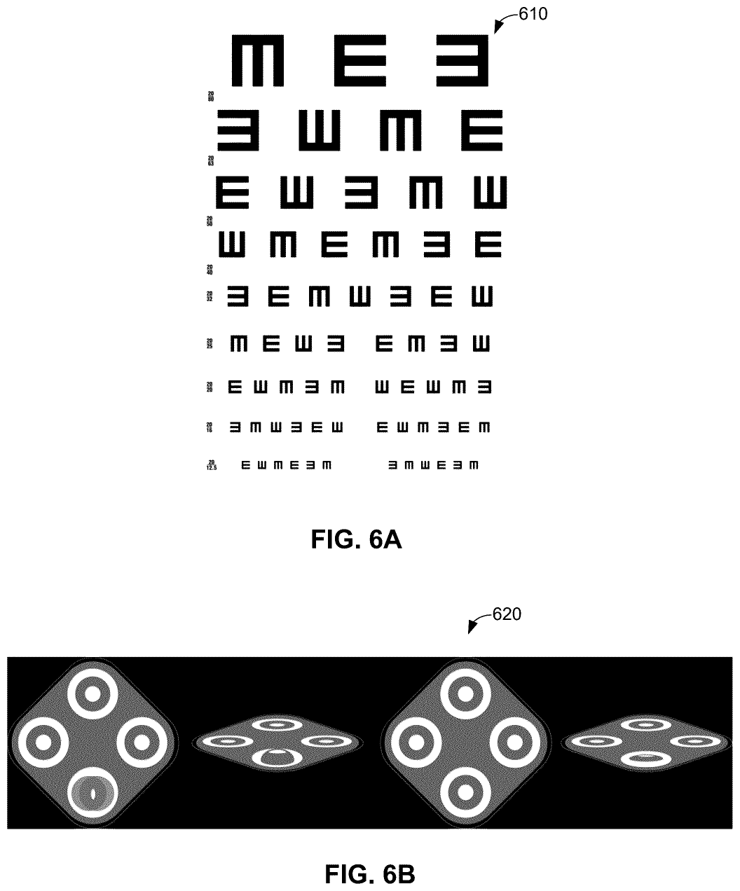

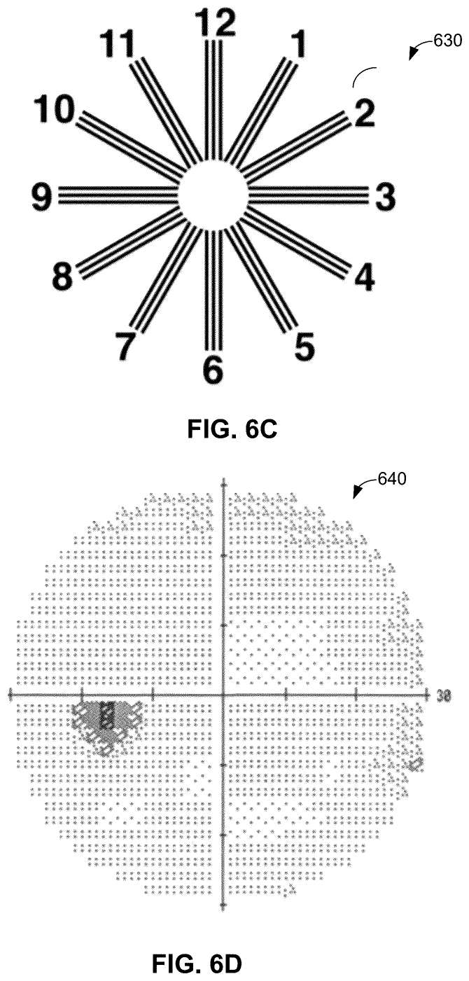

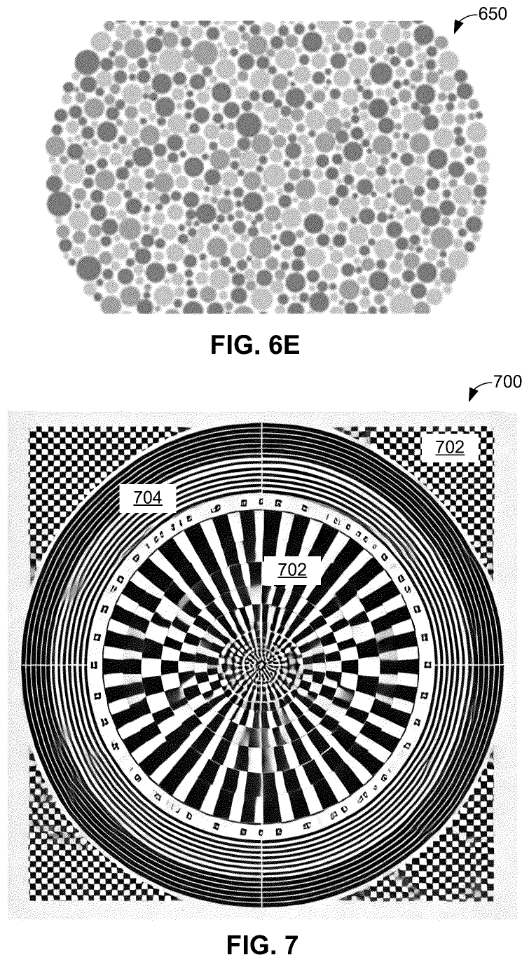

In some embodiments of the present disclosure, a vision test is implemented in a headset device 140D configured to display a user interface creating a three-dimensional (3D) virtual environment. Examples of a vision test implemented in the 3D virtual environment include, but are not limited to a visual acuity test, a visual field test, a visual depth test, a color blindness test, a retinoscopy, a test for stereopsis, a refraction test, an astigmatism test, and a contact lens exam. FIG. 6A is an example tumbling E chart 610 applied in a visual acuity test, in accordance with some embodiments. FIGS. 6B, 6C, 6D, and 6E are example patterns 620, 630, 640, and 650 applied in a stereopsis test, an astigmatism test, a visual field test, and a color blindness test, in accordance with some embodiments.

FIG. 7 is another example visual pattern 700 applied to test visual acuity and astigmatism, in accordance with some embodiments. The visual pattern 700 integrates a grid pattern 702 and concentric rings 704. The grid pattern 702 may include evenly spaced horizontal and vertical lines, creating a checkerboard pattern. The grid pattern 702 may be configured to identify distortions in straight lines, which can indicate issues with visual acuity and astigmatism. The concentric rings 704 may expand outward from a center of the visual pattern 700 and can assist in detecting radial distortions, which are common indicators of astigmatism. The visual pattern 700 may be depicted in high-contrast black and white, which ensures maximum clarity and reduces the potential for color-related distortions, making it easier to detect any visual impairment or defect.

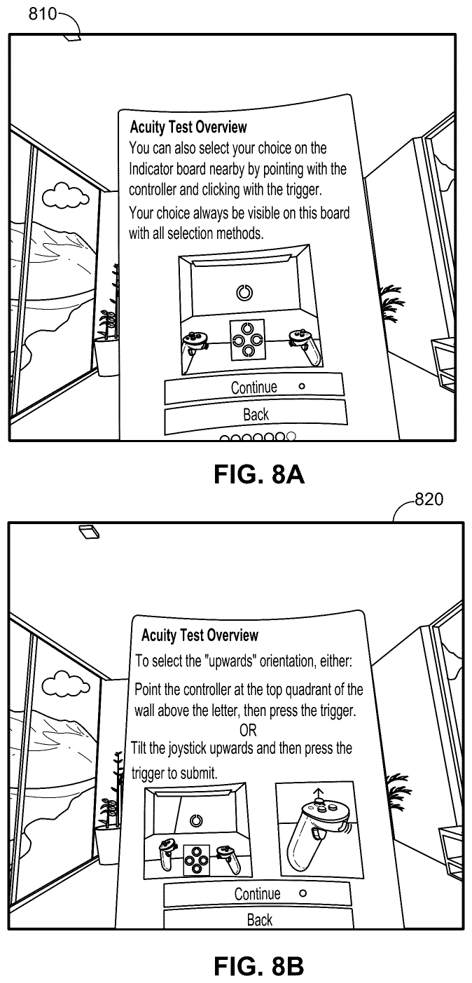

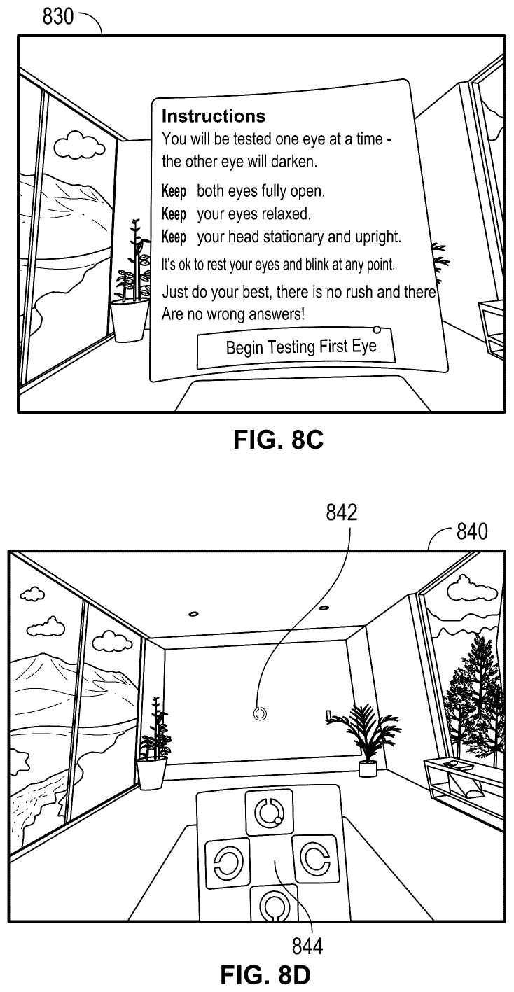

FIGS. 8A-8D include four diagrams of example graphical user interfaces 810, 820, 830, and 840 rendered to determine a visual acuity score in a virtual environment created by a headset device 140D, in accordance with some embodiments. The user interface 810 may display an information page including instructions on controlling a headset device 140D to select one of a plurality of optotype candidates to match a target optotype displayed in the virtual environment. The user interface 820 may display an information page including two optional ways of using a controller 390 (FIG. 3) to select the one of the plurality of optotype candidates. The user interface 830 may display an information page including general guidelines on a visual acuity assessment process. The user interface 840 may display an optotype 842 that is projected on a screen that has a first distance L1 from a user's position in the virtual environment. In a second distance L2 near the user, a selection panel 844 including a plurality of optotype candidates may be displayed, prompting the user to select one of the optotype candidates that matches the optotype 842. In some embodiments, in response to a user selection of the one of the optotype candidates, the optotype 842 displayed in the first distance L1 may be updated with a new optotype 842. Further, in some embodiments, the new optotype 842 may spin at a fast rate for a shortened duration of time (e.g., 2 seconds), before it settles in place of the original optotype 842. In an example, the optotype 842 may spin and gradually shrink in size during the shortened duration of time.

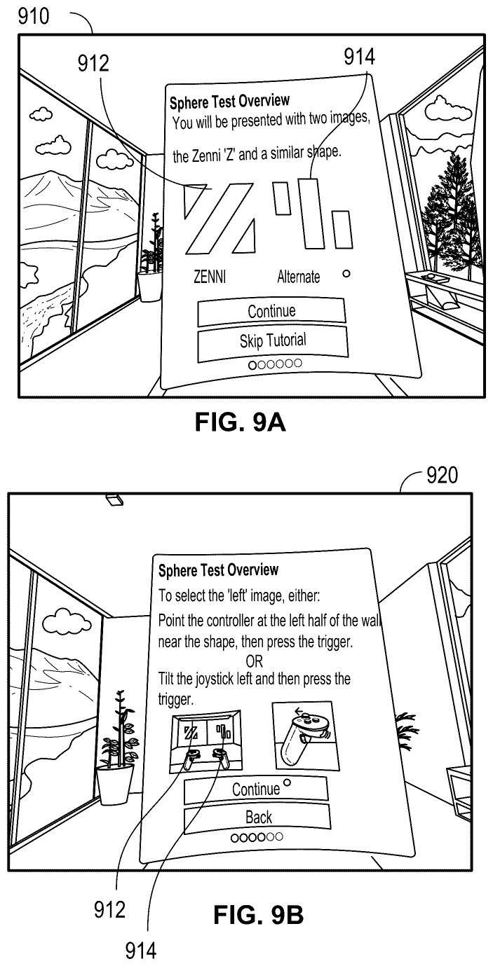

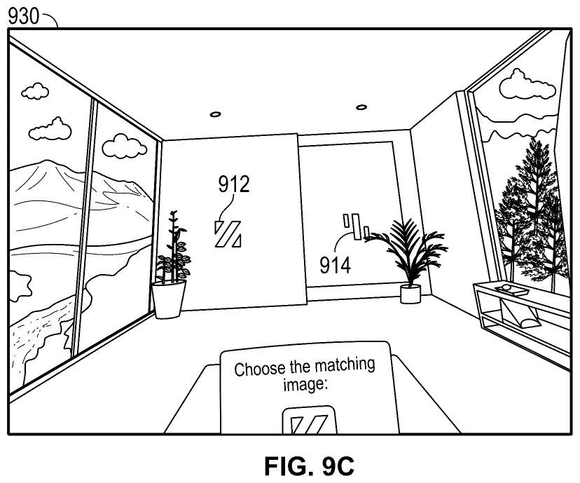

FIGS. 9A-9C include three diagrams of example graphical user interfaces 910, 920, and 930 rendered to determine a nearsighted or farsighted power in a virtual environment created by a headset device 140D, in accordance with some embodiments. The user interface 910 may display an information page explaining that two target optotypes 912 and 914 may be displayed in the virtual environment. The user interface 920 may display an information page including two optional ways of using a controller 390 (FIG. 3) to select one of the two target optotypes 912 and 914. The user interface 930 may display two target optotypes 912 and 914 that may be projected on a screen that has a first distance L1 from a user's position in the virtual environment. In this example, the target optotype 912 located on the left is highlighted (e.g., by being displayed in a colored background). In a second distance L2 near the user, a confirmation panel may be displayed, prompting the user to select one of the two target optotypes 912 and 914. In some embodiments, in response to a user selection of the one of the two target optotypes 912 and 914, the two target optotypes 912 and 914 displayed in the first distance L1 may be updated with a new pair of two target optotypes 912 and 914. Further, in some embodiments, each optotype 912 or 914 may spin at a fast rate for a shortened duration of time (e.g., 2 seconds), before it settles in place of the original optotype 912 or 914. In an example, the optotype 912 or 914 may spin and gradually shrink in size during the shortened duration of time.

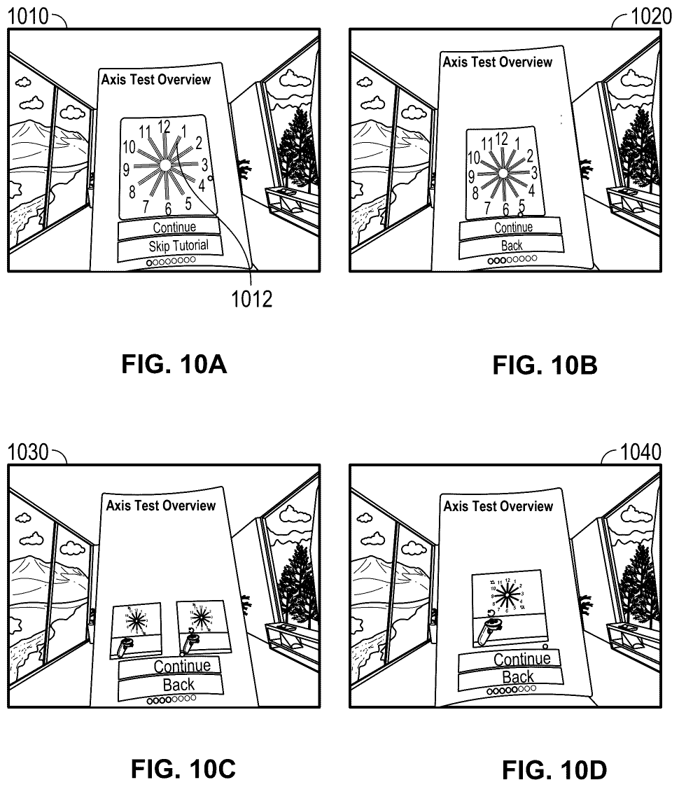

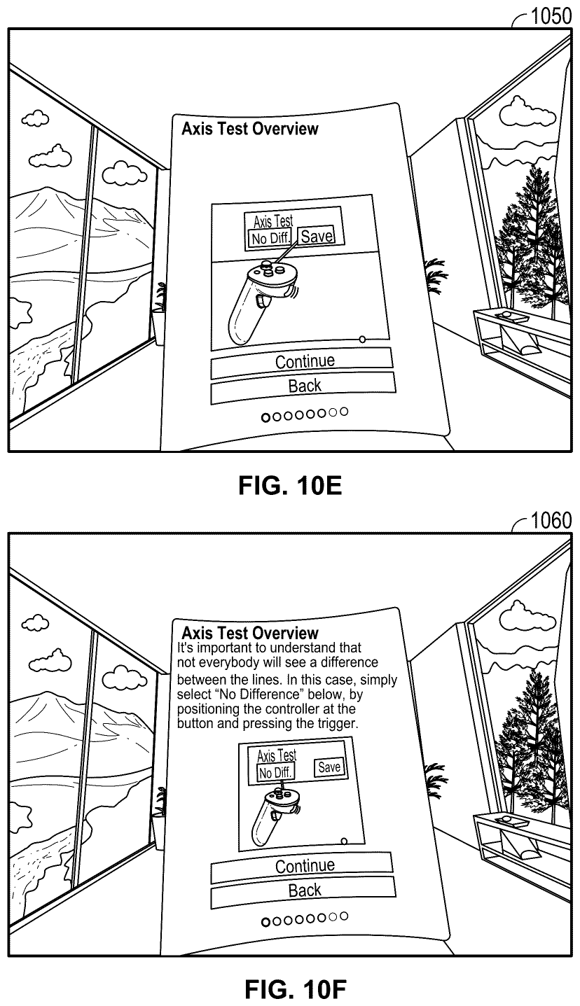

FIGS. 10A-10F include six diagrams of example graphical user interfaces 1010, 1020, 1030, 1040, 1050, and 1060 rendered to determine eye stigmatism in a virtual environment created by a headset device 140D, in accordance with some embodiments. The user interface 1010 may display an information page explaining that a clock diagram of converging numbered lines 1012 (which is a type of optotype) is displayed in the virtual environment. For example, the user interface 1010 may include a message, e.g., “You will be presented with a clock diagram of converging numbered lines. ” The user interface 1020 may display an information page explaining what is selected on the clock diagram of converging numbered lines 1012 displayed in the virtual environment. For example, the user interface 1010 may include a message, e.g., “Your task is to identify if any of these sets of lines appear clearer, crisper, or darker than other.” The user interface 1030 may display an information page including two optional ways of using a controller 390 (FIG. 3) to select lines on the clock diagram of converging numbered lines 1012. For example, the user interface 1010 may include a message, e.g., “Make a selection by either pointing the controller 390 at the lines on the clock, then pressing the trigger and Rotating the joystick to move the indicator arrows around the clock. ” The user interface 1040 may display an information page illustrating an embodiment having equally clear lines on the clock diagram of converging numbered lines 1012. For example, the user interface 1010 may include a message, e.g., “If two sets of neighboring lines seem to both stand out as equally clear, you can move the indicator arrows to a halfway point between those lines.” Referring to FIG. 10E, the user interface 1050 may display an information page including an instruction using the controller 390 to submit a selection. For example, the user interface 1010 may include a message, e.g., “After selecting a set of lines, submit your choice with the ‘Done’ button below by pointing to the controller 390 at the button and pressing the trigger.” Further, referring to FIG. 10F, the user interface 1060 may display an information page including an instruction using the controller 390 to indicate that no difference is observed on the clock diagram of converging numbered lines 1012. For example, the user interface 1010 may include a message, e.g., “It's important to understand that not everybody will see a difference between the lines and In this case, simply select ‘No Difference’ below, by positioning the controller at the button and pressing the trigger.

### Assessment of Visual Processing Speed and Accuracy With Cognitive Tasks

Some implementations of this application include a VR-based computer system 300 configured to assess visual processing speed and accuracy through engaging cognitive tasks. The computer system 300 may utilize a high-resolution VR headset that may be equipped with eye-tracking sensors (e.g., eye-tracking cameras 366 in FIG. 3) and a visual assessment application to create interactive cognitive tasks within a virtual environment. Users may wear the VR headset and participate in a series of tasks that require quick visual recognition, decision-making, and motor responses. The eye-tracking sensors may continuously monitor the user's gaze direction, fixation duration, and saccadic movements, while the software analyzes these responses to measure visual processing speed and accuracy in real-time.

In some embodiments, the VR-based computer system 300 may incorporate a range of cognitive tasks, such as identifying specific objects among distractors, following moving targets, and responding to visual cues that change dynamically. These tasks may be applied to challenge the user's visual processing abilities, providing data on rapid visual recognition, swift decision-making, and precise motor responses. A user application 324 (e.g., visual assessment application 328 in FIG. 3) may process the data and evaluate parameters such as reaction time, accuracy of visual recognition, and consistency of responses. Results may be compiled into a report that provides insights into the user's visual processing speed and accuracy, highlighting any deficiencies that could indicate underlying neurological or visual processing disorders. As such, the computer system 300 may offer a dynamic, engaging, and precise approach for assessing visual processing capabilities in a controlled virtual environment.

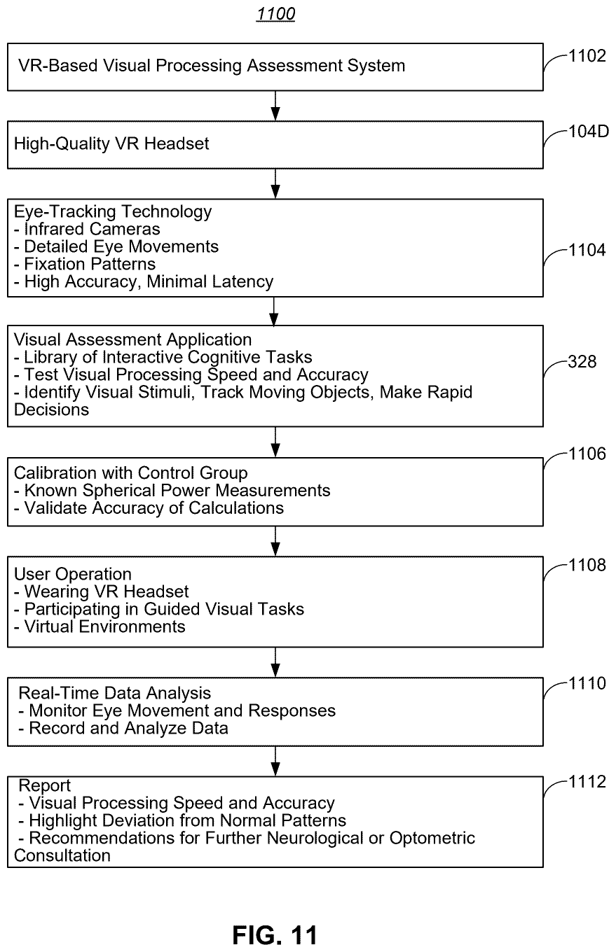

FIG. 11 is a flow diagram of an example vision test process 1100 for determining visual processing speed and accuracy of a user's visual system, in accordance with some embodiments. The VR-based computer system 300 may be configured to enable a VR-based visual processing assessment system 1102. The computer system 300 may include a VR headset 104D that includes an eye-tracking camera 366 (FIG. 3). The eye-tracking technology may include an infrared camera (e.g., camera 366) configured to capture (operation 1104) eye movements and fixation patterns with high accuracy and minimal latency. In some embodiments, when a visual assessment application 328 is executed, a library of interactive cognitive tasks may be applied to test different aspects of visual processing speed and accuracy. These tasks may include scenarios where users may be prompted to identify and respond to visual stimuli, follow and track moving objects, and make decisions based on changing visual information.

In some embodiments, when hardware components and software modules may be integrated to form the spherical power measurement system 1102, the VR-based computer system 300 may be calibrated (operation 1106) using a control group of individuals with known visual processing profiles to establish baseline performance metrics and validate the accuracy of the assessment algorithms. Users can operate (operation 1108) the calibrated computer system 300 by wearing the VR headset and participating in the guided cognitive tasks within the virtual environments. The eye-tracking camera 366 may monitor their eye movements and responses to the visual stimuli. Image or video data recorded by the camera 366 may be analyzed (operation 1110) in real time by the software modules (e.g., visual assessment application 328, data processing module 330 in FIG. 3). In some implementations, the user may receive a report 1112 outlining their visual processing speed and accuracy, highlighting any deviations from normal patterns, and providing recommendations for further neurological or optometric consultation. By these means, the computer system 140 may offer a precise, non-invasive, and user-friendly method for assessing visual processing speed and accuracy, representing a significant advancement over traditional testing techniques and providing substantial benefits for both clinical and research applications.

FIGS. 12A and 12B are flow diagrams of two example vision test processes 1200 and 1250 for determining a visual processing performance factor 1220 of eyes of a user 120, in accordance with some embodiments, respectively. FIG. 13 is a diagram of an example gaze path 1300 on which a gaze of a user's eyes approaches a visual stimulus, in accordance with some embodiments. The vision test processes 1200 and 1250 may be implemented by a computer device 140 (e.g., a headset device 140D) that may further include one or more processors, memory storing instructions to be executed by the one or more processors, and a HMD 312A. The computer device 140 may execute a user application 324 (e.g., a visual assessment application 328 in FIG. 3) configured to enable a virtual vision test and generate a VR user interface 1202 corresponding to a 3D virtual environment. Referring to FIG. 12A, in some embodiments, while a sequence of visual stimuli 1204 are displayed on the user interface 1202, the computer device 140 may obtain a sequence of eye images 1206 of two eyes of the user 120 associated with the computer device 140. The sequence of visual stimuli 1204 corresponds to a sequence of stimulus positions 1208 in the 3D virtual environment. The computer device 140 may determine a sequence of 3D gaze positions 1210 of the eyes in the 3D virtual environment based on the sequence of eye images 1206. The visual processing performance factor 1220 may be determined for the user 120 based on the sequence of stimulus positions 1208 and the sequence of 3D gaze positions 1210.

In some embodiments, the computer device 140 may adaptively determine one or more display parameters 1214 (e.g., a font size, a foreground color, a brightness level, a background style, and caption parameters) for displaying media content 1212 on the HMD 312A based on the visual processing performance factor 1220.

In some embodiments, the computer device 140 may determine the sequence of 3D gaze positions 1210 of the eyes by extracting a region of interest (ROI) image 1216 of the two eyes from each eye image to form a sequence of ROI images 1216 based on the sequence of eye images 1206. A focus tracking model 1218 may be applied to process the sequence of ROI images 1216 jointly and generate an output vector 1222 including the sequence of 3D gaze positions 1210. Alternatively, in some embodiments, each eye image 1206 may be captured at a respective time t, and the computer device 140 may determine the sequence of 3D gaze positions 1210 of the eyes by generating an ROI image 1216 of the two eyes in the respective eye image 1206 and determining a respective 3D gaze position 1210 individually based on the ROI image 1216.

In some embodiments, for each eye image 1206 captured at a respective time, the computer device 140 may identify a left eye center 1224L and a right eye center 1224R, determine a left line of sight 1226L extending from the left eye center 1224L and a right line of sight 1226R extending from the right eye center 1224R, and determine a respective 3D gaze position 1210 as an intersection point of the left line of sight 1226L and the right line of sight 1226R.

In some embodiments, the visual processing performance factor further includes at least one of a visual processing speed 1228 and a visual processing accuracy 1230. The visual processing speed 1228 may correspond to a delay between a first time when a new visual stimulus 1204 appears on the user interface 1202 and a second time when the eyes' 3D gaze position 1210 lands on or substantially close to the stimulus location 1208 of the new visual stimulus 1204. In some embodiments, when the eyes' 3D gaze position 1210 fails to land on or substantially close to the stimulus location 1208, e.g., within a predefined time duration measured from the first time, the visual processing accuracy 1230 is substantially low and does not satisfy a visual processing tolerance. In some embodiments, based on the visual processing performance factor 1220, the computer device 140 may determine a visual processing deficiency condition 1232.

In some embodiments, each pair of two successive visual stimuli of the sequence of visual stimuli 1204 has a respective stimulus position change. A relationship 1234 of the visual processing performance factor 1220 and the respective stimulus position change may be determined.

In some embodiments, for each stimulus position 1208, the computer device 140 may determine the visual processing performance factor 1220 by identifying a respective set of one or more 3D gaze positions 1210 that satisfy a predefined response criterion 1240 (FIG. 13). Referring to FIG. 13, when a first visual stimulus newly shows up at a stimulus position 1208A, the 3D gaze position 1210 may move in the 3D virtual environment to follow the first visual stimulus. For example, the 3D gaze position 1210 may move among the positions P1, P2, . . . , and P11 successively to get close to the stimulus position 1208A. The 3D gaze position 1210 may not land perfectly on the stimulus position 1208A of the first visual stimulus. Instead, the 3D gaze position 1210 may move around the stimulus position 1208A. Further, in some embodiments, the predefined response criterion 1240 may require that, for each stimulus position 1208, the respective set of one or more 3D gaze positions 1210 may be located within a respective physical range 1302 surrounding the respective stimulus position 1208. The respective physical range 1302 may be associated with a depth of the respective stimulus position 1208. The further away the respective visual stimulus 1204, the large the respective physical range 1302. In an example, the stimulus position 1208A of the first visual stimulus is 20 feet away from the user 120, and the respective physical range 1302 associated with the predefined response criterion 1240 is set to be 1 foot. Under some circumstances, the predefined response criterion 1240 may be satisfies, when the 3D gaze position 1210 stabilizes within 1 foot of the stimulus position 1208A within three seconds of displaying the first visual stimulus at the stimulus position 1208A.

In some embodiments, for each visual stimulus 1204, the stimulus position 1208 may correspond to a series of respective gaze points 1210 (e.g., the first stimulus position 1208A may correspond to gaze points P1-P11). Each stimulus position 1208 corresponds to a stimulus time TS, and each gaze position 1210 corresponds to a focal time TG. The visual processing performance factor 1220 may be determined based on the stimulus time TS of each of a subset of stimuli 1204 and the focal times TG of the respective set of one or more 3D gaze positions 1208 corresponding to the respective stimulus position 1208. Additionally, in some embodiments, for each stimulus position 1208, the computer device 140 may identify a respective response time corresponding to a first gaze position (e.g., P3 in FIG. 13) having the earliest focal time among the respective set of one or more 3D gaze positions 1210, and further determine a visual processing speed 1228 based on one or more respective response times of a subset of one or more stimulus positions 1208. Referring to FIG. 13, the gaze position P3 may be selected to measure a response time to the first visual stimulus displayed at the stimulus position 1208A, because the gaze point 1210 corresponding to the gaze position P3 is the first gaze point appearing within the respective physical range 1302 of the first visual stimulus.

In some embodiments, for each stimulus position 1208 (e.g., position 1208A in FIG. 13), the computer device 140 may determine an average gaze position 1304 of the respective set of one or more 3D gaze positions 1210. A position offset 1236 may be determined based on the average gaze position 1304 and the stimulus position 1208, and the visual processing accuracy 1230 may be determined based on the position offsets 1236 of the stimulus positions 1208. In some embodiments, for each visual stimulus 1204, the respective set of one or more 3D gaze positions 1210 applied to determine the average gaze position 1304 may be located within the respective physical range 1302 surrounding the respective stimulus position 1208. For example, gaze positions P3-P7 and P9-P11 (FIG. 13) are applied to determine the average gaze position 1304. In another example (FIG. 13), gaze positions P3-P11 are applied to determine the average gaze position 1304. Alternatively, in some embodiments, for each visual stimulus 1204, the respective set of one or more 3D gaze positions 1210 applied to determine the average gaze position 1304 may include gaze positions 1210 that are started with the first gaze point (e.g., P3 in FIG. 13), which appears earliest within the respective physical range 1302 of the first visual stimulus. For example, gaze positions P3-P11 (FIG. 13) may be applied to determine the average gaze position 1304, and include the gaze point P8 that temporarily falls out of the respective physical range 1302 surrounding the stimulus position 1208A.

In some embodiments, the computer device 140 may determine a sequence of pupil sizes 1238 of at least one eye based on the sequence of eye images 1206. A focus level 1242 of the sequence of pupil sizes 1238 may be determined for the user 120. The computer device 140 may adjust the visual processing performance factor 1220 based on the focus level 1242. In some embodiments, the larger the pupil size 1238, the lower the focus level of the user 120. When the computer device 104 determines that the user 120 is not focused based on the eye images 1206 (e.g., when the focus level is lower than a focus threshold level), the computer device 104 may adjust the visual processing performance factor 1220, issue a reminder message, reduce the complexity level of subsequent visual stimuli 1204. In some embodiments, when the computer device 104 determines that the user 120 is not focused (e.g., when the pupil size 1238 is greater than a pupil threshold or when the focus level is lower than a focus threshold level), the computer device 104 may obtain a low confidence score for the visual processing performance factor 1220.

In some embodiments, the sequence of visual stimuli 1204 may include a known stimulus 1204P that is displayed sequentially at the sequence of stimulus positions 1208. The sequence of stimulus positions 1208 may correspond to different depths measured with respect to a location of the user 120 in the 3D virtual environment, and a size of the known stimulus 1204P may be adjusted based on the different depths associated with the sequence of stimulus positions. The closer the known stimulus to the user 120, the greater the size of the known stimulus.

In some embodiments, the computer device may execute a visual assessment application 328 and display a user interface 1202 to create a 3D virtual environment. While displaying a sequence of visual stimuli 1204 on the user interface 1202, the computer device 140 may obtain a sequence of eye images 1206 of two eyes of a user 120 associated with the computer device 140. The sequence of visual stimuli 1204 corresponds to a sequence of stimulus positions 1208 in the 3D virtual environment. A visual processing assessment model 1244 may be applied to receive the sequence of eye images 1206 and the sequence of stimulus positions 1208 as inputs and generate a visual processing performance factor 1220 for the user 120.

In some embodiments, the sequence of eye images 1206 correspond to a sequence of ROI images 1216 of eye regions, and the computer device 140 may extract each eye image 1206 from tracking images captured by an eye tracking camera 366.

In some embodiments, the computer device 140 may extract an image feature 1246 from each eye image 1206, and generate a model input feature 1248 by arranging the image features 1246 of the sequence of eye images 1206 and the sequence of stimulus positions 1208 according to a predefined input data structure. The model input feature 1248 may be fed into the visual processing assessment model 1244.

### Controller Actuation in Three-dimensional Virtual Vision Tests

Some implementations of this application include a VR-based computer system 300 configured to assess tactile visual response by simulating various textures and depths within a virtual environment. The computer system 300 may utilize a VR headset integrated with precision eye-tracking technology and specialized haptic feedback gloves or controllers. The VR headset generates immersive 3D environments where users can interact with virtual objects that simulate different textures and depths. The eye-tracking camera 366 may monitor the user's gaze direction and focus points, while the haptic feedback devices provide tactile sensations corresponding to the visual stimuli, facilitating assessment of the user's ability to visually perceive and physically interact with various textures and depths.

In some embodiments, the VR-based computer system 300 may incorporate a range of interactive tasks, such as exploring virtual surfaces with different textures (smooth, rough, sticky) and engaging with objects that require depth perception (reaching into containers of varying depths, stacking items). These tasks may be applied to challenge the user's visual-tactile integration, providing data on them to rely on both visual cues and haptic feedback to accurately perceive and interact with the virtual environment. A user application 324 (e.g., visual assessment application 328 in FIG. 3) may process the data in real time, and evaluate the user's tactile visual response, including reaction time, accuracy of texture identification, and depth perception. Results may be compiled into a report that provides insights into the user's tactile visual response capabilities, identifying any deficiencies that could indicate conditions such as sensory integration disorders or visual-tactile dysfunctions. As such, the computer system 300 may offer a dynamic, engaging, and precise approach for assessing tactile visual response in a controlled virtual environment.

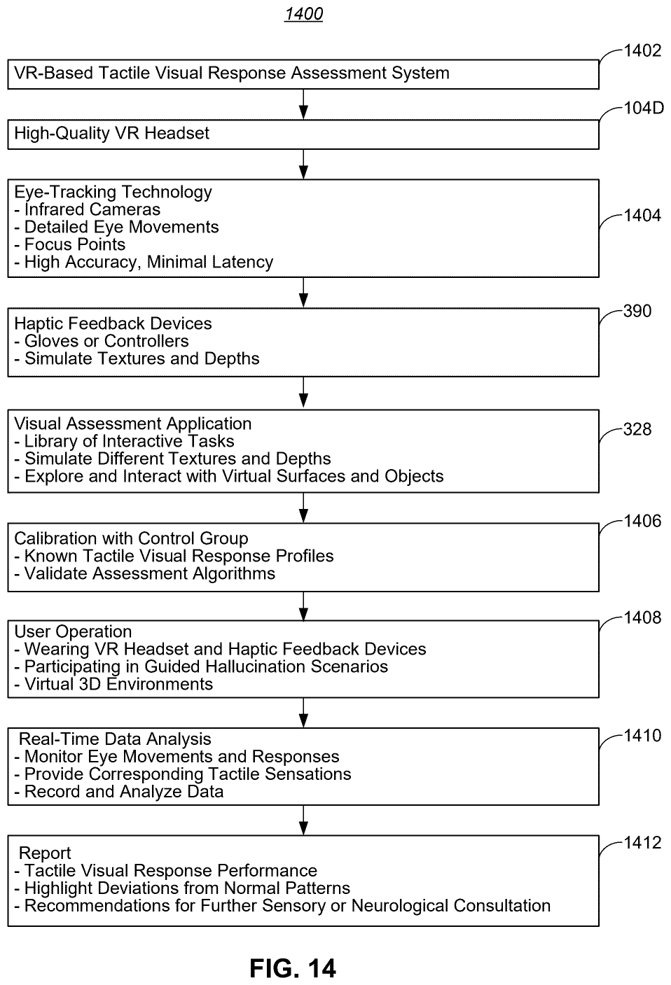

FIG. 14 is a flow diagram of an example process 1400 for actuating controllers in synchronization with a vision test, in accordance with some embodiments. The VR-based computer system 300 may be configured to enable a VR-based tactile-aided visual response assessment system 1402. The computer system 300 may include a VR headset 104D that includes an eye-tracking camera 366 (FIG. 3) paired with haptic feedback gloves or controllers 390 (FIG. 3). The eye-tracking camera 366 may include an infrared camera (e.g., camera 366) configured to capture (operation 1404) eye movements and focus points with high accuracy and minimal latency. In some embodiments, the haptic feedback devices 390 may simulate a wide range of textures and depths to provide realistic tactile sensations. In some embodiments, when a visual assessment application 328 is executed, a library of interactive tasks may be applied to simulate different textures and depths, including scenarios where users may explore and interact with virtual surfaces and objects.

In some embodiments, when hardware components and software modules may be integrated to form the VR-based tactile visual response assessment system 1402, the VR-based computer system 300 may be calibrated (operation 1406) using a control group of individuals with known tactile visual response profiles to establish baseline performance metrics and validate the accuracy of the assessment algorithms. In some embodiments, users may operate (operation 1408) the system by wearing the VR headset and haptic feedback devices, participating in the guided interactive tasks within the virtual environments. The eye-tracking camera 366 may monitor their eye movements and responses to the visual stimuli, and the haptic feedback devices may provide corresponding tactile sensations in synchronization with the camera 366. Image or video data recorded by the camera 366 may be analyzed (operation 1410) in real time by the software modules (e.g., visual assessment application 328, data processing module 330 in FIG. 3). In some implementations, the user may receive a report 1412 outlining their tactile visual response performance, highlighting any deviations from normal patterns, and providing recommendations for further sensory or neurological consultation. By these means, the computer system 140 may offer a precise, non-invasive, and user-friendly method for assessing tactile visual response, representing a significant advancement over traditional testing techniques and providing substantial benefits for both clinical and research applications.

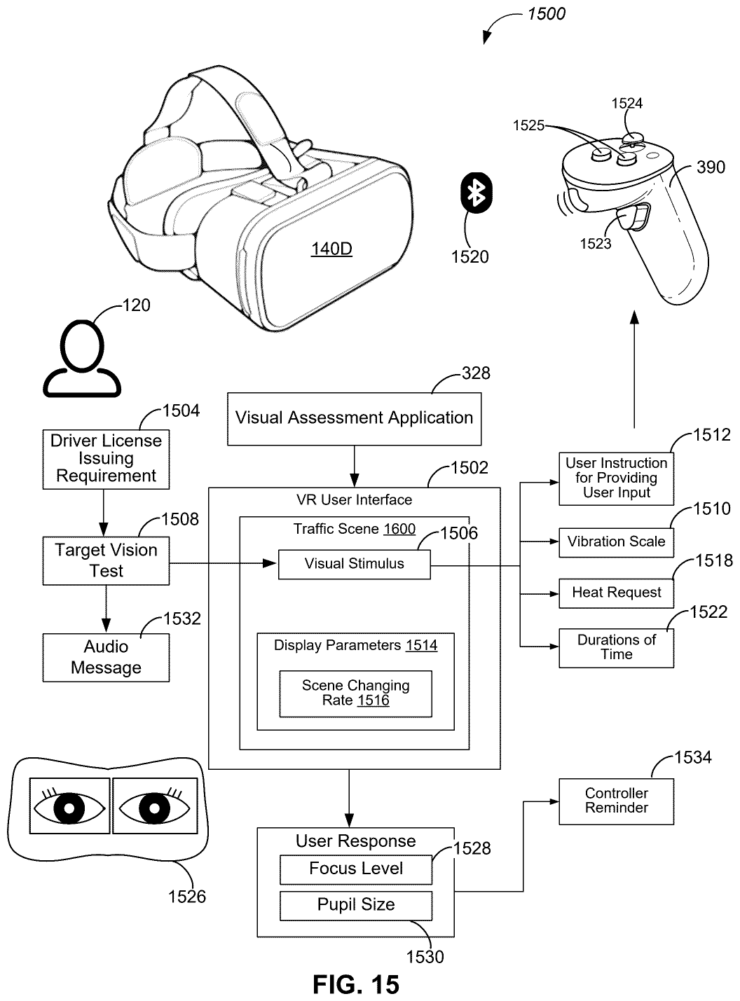

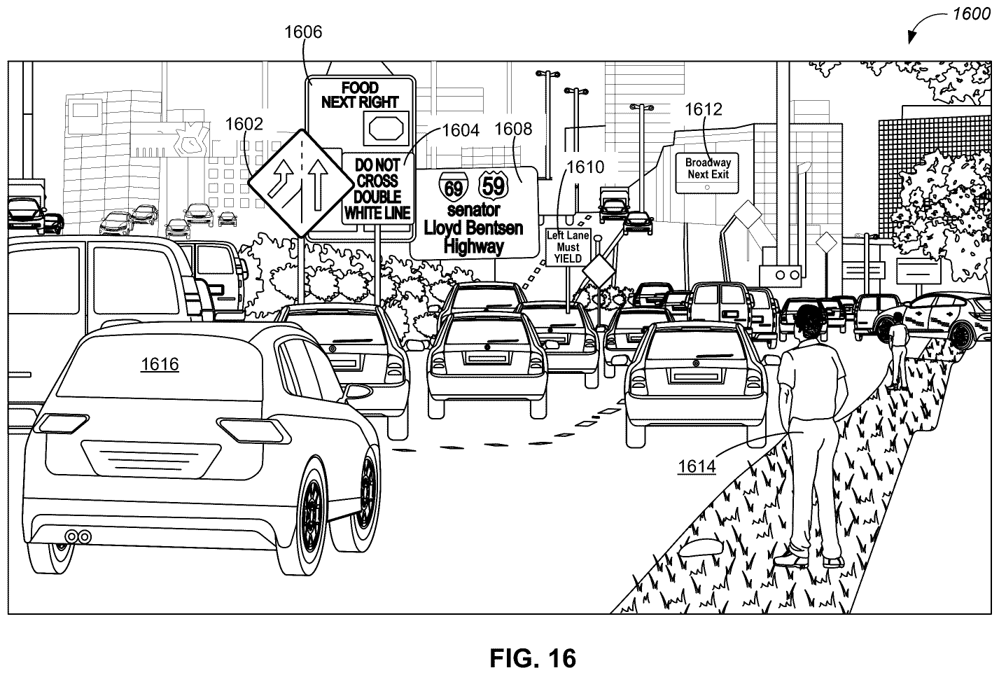

FIG. 15 is a flow diagram of an example vision test process 1500 for facilitating a vision test with a controller 390 in a 3D virtual environment, in accordance with some embodiments, and FIG. 16 is an example traffic scene 1600 enabled in a virtual environment for a vision test facilitated by the controller 390, in accordance with some embodiments. The vision test process 1500 may be implemented by a computer device 140 (e.g., a headset device 140D). The computer device may further include one or more processors, memory storing instructions to be executed by the one or more processors, and a HMD 312A. The computer device 140 may execute a user application 324 (e.g., a visual assessment application 328) configured to enable the vision test and generate a VR user interface 1502 corresponding to a 3D virtual environment. This vision test process 1500 may be facilitated by a controller 390. The controller 390 is a handheld device for interacting with the user application 324 (e.g., video games) by providing input commands to the computer device 140. In some embodiments, the controller 390 may include one or more of buttons 1523, analog sticks 1524, and triggers 1525, and allow a user 120 to control characters, vehicles, or navigate the 3D virtual environment. For example, the user 120 of the headset device 140D may be prompted to apply the controller 390 to provide user inputs during vision tests (e.g., in FIGS. 8A-8B and 10C-10F). In some embodiments, the controller may further include one or more of: motion sensors, haptic feedback, and wireless connectivity, enhancing user experience with more immersive and responsive control. Examples of the motion sensors used in the controller 390 include, but are not limited to, an accelerometer, a gyroscope, a magnetometer, an IR sensor, a touch sensor, a proximity sensor, a pressure sensor. In some implementations, the controller 390 may be ergonomically shaped to offer comfort during extended VR sessions while providing precision and functionality across a wide range of user applications 324.

In some embodiments, a communication link may be established between the computer device 140 (e.g., the headset device 140D) and the controller 390 using a wired connection (e.g., universal serial bus (USB)), Bluetooth communication, WiFi Direct communication, radio frequency communication, IR signal exchange, or other proprietary wireless communication. The controller 390 may be held by a user 120 associated with the computer device 140. The computer device 140 displays a VR user interface 1502 based on a driver license issuing requirement 1504 to implement a target vision test 1508 in a 3D virtual environment, the VR user interface 1502 including a moving traffic scene 1600 (e.g., on which one or more visual stimuli 1506 are displayed). One or more actuators of a controller 390 may be driven in synchronization with displaying the VR user interface 1502. In some embodiments, the one or more actuators of a controller 390 may vibrate the controller 390 with a vibration scale 1510.

In some embodiments, the computer device 140 may generate a user instruction 1512 to request the user 120 to provide a user input via the controller 390 in response to displaying the one or more visual stimuli 1506. Further, in some embodiments, the computer device 140 may identify a presumed speed of a virtual vehicle (e.g., vehicle 1616 in FIG. 16) associated with the vison test 1508, set the vibration scale 1510 based on the presumed speed, and set a scene changing rate based on the presumed speed. During an extended duration of time, the moving traffic scene 1600 is dynamically generated and updated based on the scene changing rate 1516, and the controller 390 is dynamically vibrated based on the vibration scale 1510.

In some embodiments, the computer device 140 may add a virtual road bump effect to the moving traffic scene 1600. For example, the computer device 140 may set a road bumpiness level, and the vibration scale 1510 of the controller 390 is set accordingly based on the road bumpiness level. The scene changing rate 1516 may also be set based on the road bumpiness level. During a shortened duration of time, the moving traffic scene 1600 may be generated based on the scene changing rate 1516, and the controller 390 may be vibrated based on the vibration scale 1510. Stated another way, the computer device 140 may synchronize display parameters 1514 of the HMD 312A and the vibration scale 1510 of the controller 390. The display parameters 1514 of the HMD 312A may include the scene changing rate 1516 associated with the road bumpiness level, and the vibration scale 1510 may include a vibration speed and a vibration amplitude of the controller 390.

In some embodiments, the computer device 140 may generate a heat request 1518, and the one or more actuators of a controller 390 are configured to heat the controller 390 held by the user in response to the heat request 1518.

In some embodiments, the one or more actuators of the controller 390 are driven to send a reminder 1534 to the user 120 indicating a traffic situation. Further, in some embodiments, the computer device may obtain a sequence of eye images 1526, and track a focus level 1528 of the user 120 based on the sequence of eye images 1526 while displaying the VR user interface 1502. The one or more actuators of the controller 390 may be driven in accordance with a determination that the focus level 1528 of the user 120 drops below a predefined focus level. Further, in some embodiments, the computer device 140 may track the focus level 1528 of the user 120 by determining a pupil size 1530 for each of the sequence of eye image 1526. The focus level 1528 may be tracked based on the pupil size 1530 of each eye image 1526.

In some embodiments, the larger the pupil size 1530, the lower a focus level 1528 of the user 120. When the computer device 104 determines that the user 120 is not focused (e.g., when the pupil size 1530 is greater than a pupil threshold or when the focus level 1530 is lower than a focus threshold level), the computer device 104 may obtain a low confidence score for the user response, issue a controller reminder 1534, or reduce the complexity level of subsequent visual stimuli 1506.

In some embodiments, the computer device 140 may determine a disturbance associated with the moving traffic scene based on the driver license issuing requirement 1504. Based on the disturbance, the computer device 140 may play an audio message 1532 in synchronization with driving the one or more actuators of the controller 390 and displaying the VR user interface 1502.

In some embodiments, the driver license issuing requirement 1504 includes a predefined duration of time 1522. The computer device may determine that the moving traffic scene 1600 has been displayed for the predefined duration of time 1522. In accordance with a determination that the moving traffic scene 1600 has been displayed for the predefined duration of time 1522, the one or more actuators of the controller 390 may be driven to remind the user of the predefined duration of time 1522. In some embodiments, the predefined duration of time 1522 may be set based on a type of the moving traffic scene 1600.

In some embodiments, the one or more visual stimuli 1506 include a plurality of visual stimuli, and the driver license issuing requirement 1504 includes a respective duration of time 1522 for each of the plurality of stimuli 1506. For each of the plurality of stimuli 1506, the one or more actuators of the controller 390 may be driven, in accordance with a determination that a length of displaying the moving traffic scene 1600 has reached the respective duration of time 1522 and that no user response (e.g., a user press on the button 1523, a user push onto the analog stick 1524) to the respective stimulus 1506 has been received.

Some implementations of this application are directed to applying a controller 390 390 and a headset device 140D jointly in a target vision test 1508. A communication link 1520 is established between the headset device 140D and a controller 390 held by a user 120 associated with the headset device 140D. The headset device 140D may execute a media play application to enable a 3D user interface 1502, and display media content on the 3D user interface 1502. The headset device 140D obtains media metadata associated with the media content, generate a controller instruction based on the media metadata, and apply the controller instruction to drive one or more actuators of a controller 390 in synchronization with the media content. In some embodiments, the controller instruction includes a vibration scale, and the one or more actuators of the controller 390 are configured to vibrate the controller 390 with the vibration scale. In some embodiments, the one or more actuators of a controller 390 are configured to heat the controller 390 held by the user in response to the controller instruction.

Referring to FIG. 16, a target vision test 1508 may be implemented via the traffic scene 1600, and the visual assessment application 328 may execute the vision test 1508 and facilitate issuance or update of a driver license. The computer device 140 may obtain an instruction to implement a target vision test 1508. In accordance with a determination that the target vision test 1508 corresponds to a driver license issuing requirement 1504, a VR user interface 1502 may be loaded to create a 3D VR environment. The VR user interface 1502 may include the virtual traffic scene 1600, displaying a plurality of traffic signs 1602-1612 at a plurality of distances.

In some embodiments, the computer device 140 may display a plurality of traffic related objects in the virtual traffic scene 1600, the traffic related objects including one or more of: a traffic light, a pedestrian 1614, and a vehicle 1616. At least one of the traffic related objects may be moving in the virtual traffic scene. When a user associated with the HMD 312A takes the target vision test 1508, his or her visual capabilities (e.g., visual acuity, red and green traffic light recognition, visual response time) are tested in a dynamic traffic environment, allowing a government agency (e.g., Department of Motor Vehicle (DMV)) to issue driver licenses in a more reliable manner.

In an example, the traffic signs 1602, 1604, 1606, 1608, 1610, and 1612 are arranged at increasing distances. Each traffic sign is displayed with a set of respective display parameters 1514 (FIG. 15), such as a font size, a foreground color, a brightness level, and a background style. The user 120 associated with the HMD 312A may be prompted to identify what is displayed on each traffic sign. In some embodiments, the controller 390 may be controlled to vibrate with the vibration scale 1510 and remind the user 120 of providing a user input in response to a certain traffic sign. In some embodiments, a light condition of the virtual traffic scene 1600 is adjusted to test whether the user may still recognize what is displayed on each traffic sign. For example, the light condition may correspond to a sunset time, and the user may be prompted to recognize what is displayed on each traffic sign.

### Assessment of Visual Endurance

Some implementations of this application include a VR-based computer system 300 configured to test visual endurance by simulating long-duration visual tasks. The computer system 300 may utilize a high-resolution VR headset that may be equipped with eye-tracking sensors (e.g., eye-tracking cameras 366 in FIG. 3) and a visual assessment application to create extended visual scenarios. Users may wear the VR headset and engage in a series of tasks that require sustained visual attention and focus over prolonged periods. The eye-tracking camera 366 may monitor the user's gaze direction, blink rate, and fixation stability, while the software analyzes these responses to assess visual endurance, including metrics such as visual fatigue, attention drift, and overall performance over time.

In some embodiments, the VR-based computer system 300 may incorporate a range of long-duration tasks, such as reading extensive passages of text, identifying subtle changes in complex visual scenes, and performing repetitive visual-motor tasks that mimic real-world activities like assembly line work or detailed craftwork. These tasks may be applied to challenge the user's ability to maintain visual focus and accuracy over extended periods, simulating conditions that test the limits of visual endurance. A user application 324 (e.g., visual assessment application 328 in FIG. 3) may process the data in real time, and evaluate parameters such as sustained visual attention, fatigue onset, and performance degradation. Results may be compiled into a report that provides insights into the user's visual endurance capabilities, identifying any deficiencies that could indicate conditions such as digital eye strain, chronic fatigue syndrome, or other visual endurance impairments. As such, the computer system 300 may offer a dynamic, engaging, and precise approach for assessing visual endurance in a controlled virtual environment.

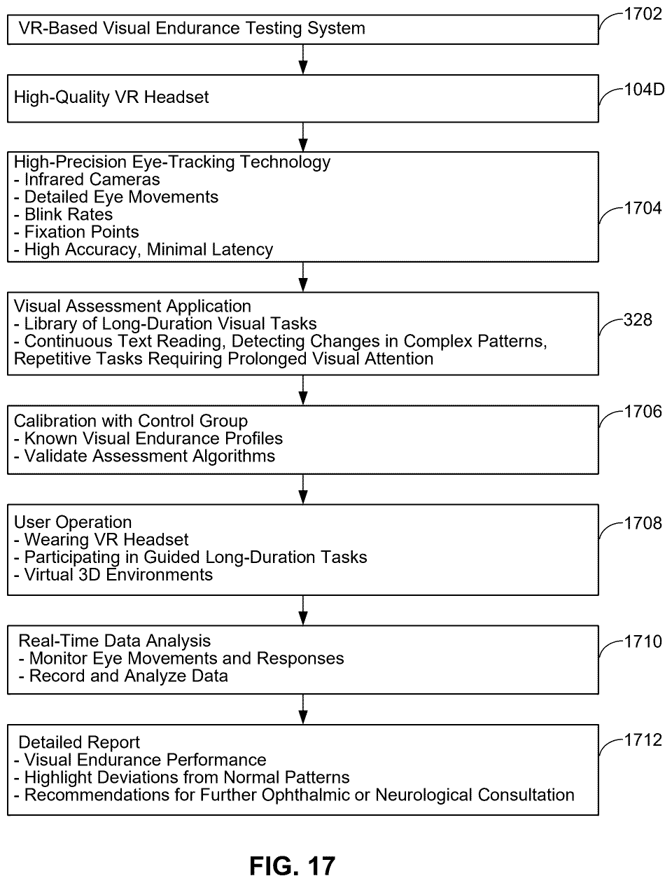

FIG. 17 is a flow diagram of an example vision test process 1700 for assessing visual endurance of a user's visual system, in accordance with some embodiments. The VR-based computer system 300 may be configured to enable a VR-based visual endurance testing system 1702. The computer system 300 may include a VR headset 104D that includes an eye-tracking camera 366 (FIG. 3). The eye-tracking technology may include an infrared camera (e.g., camera 366) configured to capture (operation 1704) eye movements, blink rates, and fixation points with high accuracy and minimal latency. In some embodiments, when a visual assessment application 328 is executed, a library of long-duration visual tasks may be used to test different aspects of visual endurance. These tasks include scenarios where users must read continuous text, detect changes in complex visual patterns, and perform repetitive tasks that require prolonged visual attention.

In some embodiments, when hardware components and software modules may be integrated to form the VR-based visual endurance testing system 1702, the VR-based computer system 300 may be calibrated (operation 1706) using a control group of individuals with known visual endurance profiles to establish baseline performance metrics and validate the accuracy of the assessment algorithms. In some embodiments, users may operate (operation 1708) the system by wearing the VR headset and participating in the guided long-duration tasks within the virtual environments. The eye-tracking camera 366 may monitor their eye movements and responses to the visual stimuli. Image or video data recorded by the camera 366 may be analyzed (operation 1710) in real time by the software modules (e.g., visual assessment application 328, data processing module 330 in FIG. 3). In some implementations, the user may receive a report 1712 outlining their visual endurance performance, highlighting any deviations from normal patterns, and providing recommendations for further ophthalmic or neurological consultation. By these means, the computer system 140 may offer a precise, non-invasive, and user-friendly method for assessing visual endurance, representing a significant advancement over traditional testing techniques and providing substantial benefits for both clinical and research applications.

FIG. 18A is a flow diagram of an example vision test process 1800 for assessing an eye endurance level 1820 in a 3D virtual environment, in accordance with some embodiments. The vision test process 1800 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface corresponding to a 3D virtual environment. The computer device 140 may display a body of text 1802 on the user interface for an extended duration of time 1804. A sequence of eye images 1810 may be obtained, and each eye image 1810 may include a respective infrared image of an ROI 1806 corresponding to at least one eye. Based on the sequence of eye images 1810, the computer device 140 may determine an eye endurance level 1820 of the at least one eye of a user 120 associated with the computer device 140.

In some embodiments, the computer device 140 may select a predefined brightness level 1808 and a predefined font size 1812, and the body of text 1802 may be displayed with the predefined brightness level 1808 and the predefined font size 1812. The predefined brightness level 1808 may be substantially high, and the predefined font size 1812 may be substantially smaller, thereby expediting the associated vision test for assessing the eye endurance level 1820 of the user's eyes. For example, an ambient brightness level of the 3D virtual environment is substantially low (e.g., equal to 100 lumens per square meter (Lux)), and the predefined brightness level 1808 of the body of text 1802 is brighter than the ambient brightness level by at least a scale factor (e.g., by 100 times). As the predefined brightness level 1808 of the body of text 1802 does not match the ambient brightness level, eye strain increases. Alternatively, in another example, the ambient brightness level of the 3D virtual environment is substantially high (e.g., equal to 30,000 Lux)), and the predefined brightness level 1808 of the body of text 1802 is darker than the ambient brightness level by at least a scale factor (e.g., by 100 times).

In some embodiments, the computer device 140 may direct an infrared camera (e.g., eye-tracking camera 366 in FIG. 3) towards the at least one eye of the user 120. The infrared camera may capture a sequence of camera images 1814 including the ROI 1806 corresponding to the at least one eye. Each camera image 1814 may be cropped based on the ROI 1806 to generate the respective eye image 1810.

In some embodiments, the computer device 140 may determine the eye endurance level 1820 by applying an eye endurance model 1816 to process the sequence of eye images 1810 and generate a model output 1822 including the eye endurance level 1820. Further, in some embodiments, the model output 1822 may include a diagnosis indicator identifying a dry eye severity level 1818 associated with the eye endurance level 1820. In some embodiments, the eye endurance model 1816 may include a feature extraction model 1824 and an endurance assessment model 1826. The computer device 140 may apply the feature extraction model 1824 to extract a respective eye feature vector from each of the sequence of eye images 1810 and apply the endurance assessment model 1826 to process respective eye feature vectors of the sequence of eye images 1810 and generate the model output 1822.

In some embodiments, the eye endurance level 1820 is determined with respect to a predefined temporal length 1828 (e.g., 5 hours) that is greater than the extended duration of time 1804 (e.g., 15 minutes). Stated another way, the vision test may be implemented in a relatively shorter time duration to provide endurance information associated with a relatively long time duration. Further, in some embodiments, the computer device 140 receives an eye endurance model 1816 from a server 102 communicatively coupled to the computer device 140. At the server 102, the eye endurance model 1816 may be trained using training data including a sequence of eye images and a ground truth eye endurance level corresponding to the predefined temporal length 1828.

In some embodiments, the computer device 140 may execute a media play application 1830 to display multimedia content on the computer device 140. Execution of the media play application 1830 may be controlled based on the eye endurance level 1820. For example, based on the eye endurance level 1820, the user may reach a fatigue level of 50% within 30 minutes. Play of the multimedia content may be automatically paused in the media play application 1830 after one hour, and a reminder message may be displayed to request the user 120 to take a break before continuing to review the multimedia content.

In some embodiments, the computer device 140 may detect one or more eye blinking events and determine one or more eye blinking times 1832. The computer device 140 may determine a sequence of eye lid positions 1834 and a sequence of pupil sizes 1836. Each eye lid position 1834 corresponds to a respective pupil size 1836. Both the lid position 1834 and the respective pupil size 1836 are determined based on a respective eye image 1810. The eye endurance level 1820 may be determined based on the one or more eye blinking times 1832, the sequence of eye lid positions 1834, and the sequence of pupil sizes 1836.

Further, in some embodiments, the computer device 140 may determine the eye endurance level 1820 by tracking the on the one or more eye blinking times 1832, the sequence of eye lid positions 1834, and the sequence of pupil sizes 1836 with reference to a start time of displaying the body of text 1802. Additionally, in some embodiments, the computer device 140 may apply an eye endurance model 1816 to process the one or more eye blinking times 1832, the sequence of eye lid positions 1834, and the sequence of pupil sizes 1836 and determine the model output 1822 including the eye endurance level 1820.

In some embodiments, the computer device 140 may extract a sclera feature from each of the sequence of eye images 1810. The sclera is the white outer coating of an eye, and includes tough, fibrous tissue that extends from the cornea (the clear front section of the eye) to the optic nerve at the back of the eye. The sclera gives an eyeball its white color. An eye endurance model 1816 is applied to determine an eye dryness feature based on the respective sclera features of the sequence of eye images 1810, and the eye endurance level 1820 is determined based on respective sclera features. In some embodiments, the sclera feature indicates that the sclera is red, and the eye may have a dry eye condition that can comprise the eye endurance level 1820.

FIG. 18B is a flow diagram of an example vision test process 1850 for assessing a dry eye condition in a 3D virtual environment, in accordance with some embodiments. The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may display a visual pattern 1852 on the user interface for an extended duration of time. The computer device 140 may obtain a sequence of eye images 1810 from an eye-tracking camera 366 (e.g., a visible light camera, an infrared camera). Each eye image 1814 may include a sclera area. An eye endurance model 1816 may be applied to process the sequence of eye images 1810 and generate a model output 1822 including a dry eye indicator 1854 associated with a dry eye condition. In some embodiments, the dry eye indicator 1854 may indicate whether there is a dry eye condition and includes a dry eye severity level 1818. In some embodiments, the computer device 140 may select a predefined brightness level 1808, and the visual pattern 1852 is displayed with the predefined brightness level 1808. In some embodiments, the visual pattern 1852 may include a body of text 1802 (FIG. 18A). In some embodiments, the visual pattern 1852 may include a sequence of image frames configured to show accelerated motion. In some embodiments, the computer device 140 may crop the sequence of eye images 1810 to extract the sclera area from each eye image 1810 and generate a sequence of sclera images, and the eye endurance model 1816 is applied to generate the model output based on the sequence of sclera images.

### Assessment of Convergence Capabilities

Some implementations of this application include a VR-based computer system 300 configured to assess convergence insufficiency through dynamic focus tasks. The computer system 300 may utilize a VR headset integrated with precision eye-tracking technology and a visual assessment application configured to generate interactive visual tasks that challenge and measure the user's ability to converge their eyes effectively. Users may wear the VR headset and participate in a series of tasks that require focusing on virtual objects moving along different planes and distances. The eye-tracking sensors may continuously monitor the user's eye movements, convergence angles, and focus adjustments, while the software analyzes these responses to assess the user's convergence efficiency or highlight potential convergence insufficiency.

In some embodiments, the VR-based computer system 300 may incorporate a range of dynamic focus tasks, such as following a moving object from far to near, focusing on objects that shift rapidly between different depths, and maintaining focus on a converging target while peripheral stimuli are introduced. These tasks may be applied to simulate real-world scenarios that challenge the user's ability to maintain proper eye alignment and focus. A user application 324 (e.g., visual assessment application 328 in FIG. 3) may process the data in real time and evaluate parameters such as convergence speed, accuracy, and stability. Results may be compiled into a report that provides insights into the user's convergence performance, identifying any deficiencies that could indicate convergence insufficiency, a common binocular vision disorder that affects the ability to maintain eye alignment on near tasks. As such, the computer system 300 may offer a dynamic, engaging, and precise approach for assessing convergence insufficiency in a controlled virtual environment.

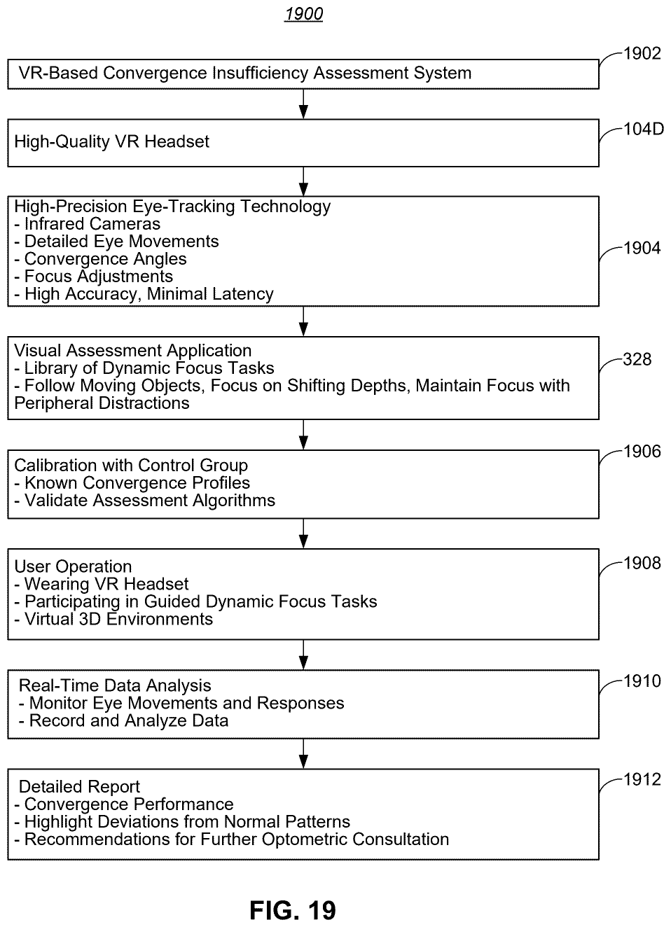

FIG. 19 is a flow diagram of an example vision test process 1900 for assessing convergence performance of a user's visual system, in accordance with some embodiments. The VR-based computer system 300 may be configured to enable a VR-based convergence insufficiency assessment system 1902. The computer system 300 may include a VR headset 104D that includes an eye-tracking camera 366 (FIG. 3). The eye-tracking technology may include an infrared camera (e.g., camera 366) configured to capture (operation 1904) eye movements, convergence angles, and focus adjustments with high accuracy and minimal latency. In some embodiments, when a visual assessment application 328 is executed, a library of dynamic focus tasks may be used to test different aspects of eye convergence. These tasks may be implemented to request the user to follow a moving object that changes distance, focus on objects that shift between various depths, and maintain focus on a converging target, while peripheral distractions may be presented.

In some embodiments, when hardware components and software modules may be integrated to form the VR-based convergence insufficiency assessment system 1902, the VR-based computer system 300 may be calibrated (operation 1906) using a control group of individuals with known convergence profiles to establish baseline performance metrics and validate the accuracy of the assessment algorithms. In some embodiments, users may operate (operation 1908) the system by wearing the VR headset and participating in the guided dynamic focus tasks within the virtual environments. The eye-tracking camera 366 may monitor their eye movements and responses to the visual stimuli. Image or video data recorded by the camera 366 may be analyzed (operation 1910) in real time by the software modules (e.g., visual assessment application 328, data processing module 330 in FIG. 3). In some implementations, the user may receive a report 1912 outlining their convergence performance, highlighting any deviations from normal patterns, and providing recommendations for further optometric consultation. By these means, the computer system 140 may offer a precise, non-invasive, and user-friendly method for assessing convergence insufficiency, representing a significant advancement over traditional testing techniques and providing substantial benefits for both clinical and research applications.

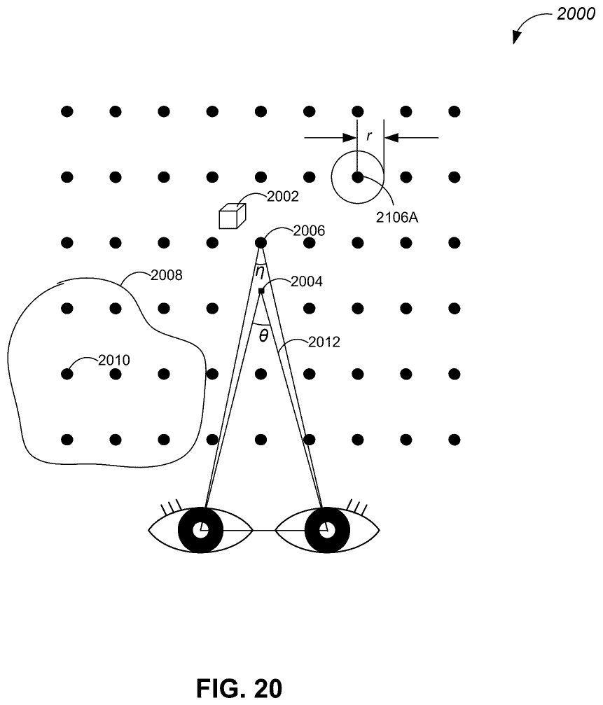

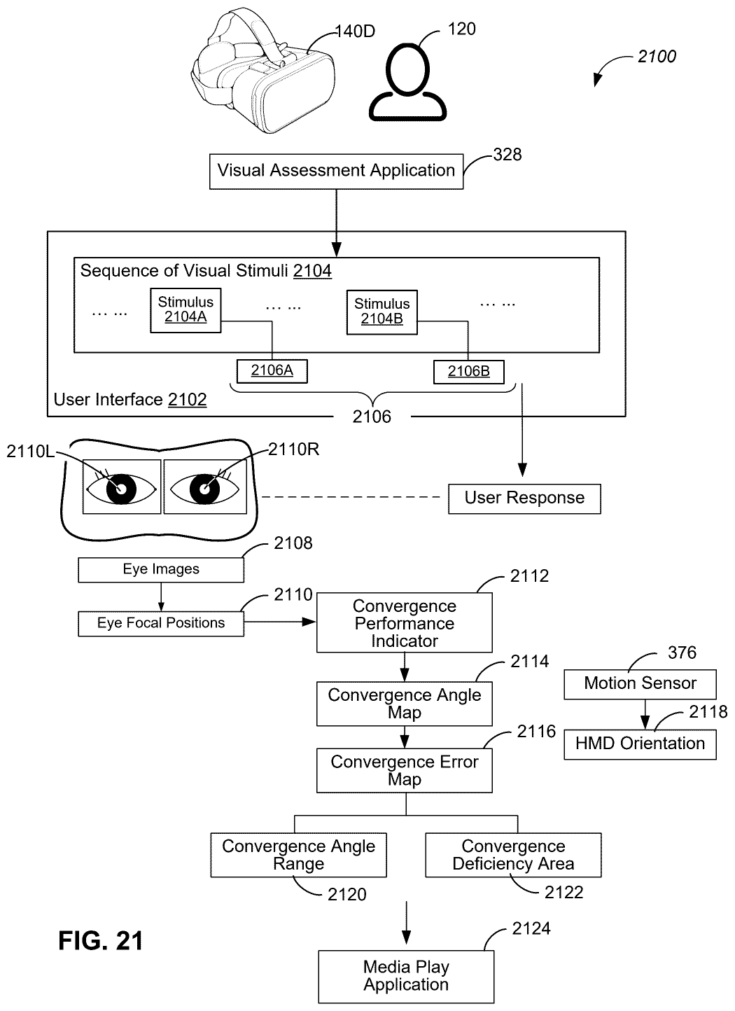

FIG. 20 is a diagram 2000 illustrating a convergence error of a user's eyes, in accordance with some embodiments. When a user's eyes focus on an object 2002 (e.g., a visual stimulus 2104 in FIG. 21), the eyes may turn towards each other, and a level of inward turning of the eyes may be represented by a convergence angle θ. The convergence angle θ is created as each eye rotates toward the nose to align respective visual axes on the object 2002 of interest, allowing for clear, single vision. The closer the object 2002, the greater the convergence angle θ. The convergence angle θ decreases as the object 2002 moves farther away. This process is an essential component of binocular vision, enabling depth perception and accurate distance judgment. Disruptions in convergence may occur and lead to double vision or eye strain, when a gaze point 2004 (also called focal point) of the eyes has an offset from an object location 2006 where the object 2002 is located. In some embodiments, disruptions in convergence may be measured by a convergence error Δ representing a difference between the convergence angle θ and an object angle η. The convergence angle θ is measured between two lines connecting the gaze point 2004 to the two eyes of the user, and the object angle η is measured between two lines connecting the object location 2006 is located to the two eyes of the user.

In some embodiments, the computer device 140 may generate a convergence error map (e.g., map 2116 in FIG. 21) quantitatively indicating convergence errors Δ for different locations in the field of view. Further, in some embodiments, the gaze point 2004 of healthy eyes may land within a tolerance range r of the object location 2006, and the tolerance range r corresponds to an error tolerance for the convergence error Δ (e.g., 0-10°). In accordance with a determination that a convergence error Δ exceeds the error tolerance, the computer device 140 may determine that convergence performance of the user's eyes at the objection location 2006 is compromised or impaired. For example, a field of view of the eyes may include a region 2008 having a plurality of locations 2010 where the respective convergence errors Δ are measured to exceed the error tolerance of the convergence error Δ. The computer device 140 may identify the region 2008 of the field of view as having impaired convergence performance, e.g., on a convergence error map.

FIG. 21 is a flow diagram of an example vision test process 2100 for assessing convergence performance of a user's eyes in a 3D virtual environment, in accordance with some embodiments. The vision test process 1200 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may display a sequence of visual stimuli 2106 on the user interface 2102. The sequence of visual stimuli 2104 (e.g., stimuli 2104A and 2104B) corresponds to a plurality of stimulus positions (e.g., locations 2106A and 2106B) distributed in the 3D virtual environment. The computer device may obtain a sequence of eye images 2108 of two eyes of a user 120 associated with the computer device 140 (e.g., a user 120 wearing a headset device 140D). A sequence of eye focal positions 2110 may be determined in the sequence of eye images 2108. The computer device 140 may determine a convergence performance indicator 2112 for the two eyes of the user 120 based on at least the sequence of eye focal positions 2110.

In some embodiments, the convergence performance indicator 2112 includes a map 2114 of convergence angles θ of the two eyes measured with respect to the plurality of stimulus positions 2106. The computer device may determine a plurality of convergence angles θ of the two eyes, and each convergence angle θ corresponds to a respective one of the plurality of stimulus positions 2106. The map of convergence angles θ of the two eyes may be generated with respect to the plurality of stimulus positions 2106.

Further, in some embodiments, for each stimulus position 2106, the computer device 140 may determine a respective convergence angle θ based on a left eye focal position 2110L of a left eye, a right eye focal position 2110R of a right eye, and a gaze point 2004 (also called a focal point). Additionally, in some embodiments, the computer device 140 may determine the left eye focal position 2110L and the right eye focal position 2110R associated with each stimulus position 2106, and derive the gaze point 2004 based on the left focal position 2110L and the right focal position 2110R. In some embodiments, when the visual stimulus 2104 is displayed (e.g., substantially close to the eyes), the eye focal positions 2110L and 2110R may shift towards each other, and a shift of the eye focal position 2110L or 2110R may be discernible in the eye images 2108. The eye focal position 2110L or 2110R and the gaze point 2004 form a triangle 2012 (FIG. 20), and the computer device 140 may further derive the convergence angle θ corresponding to each visual stimulus 2104 based on the triangle 2012. In some embodiments, for each stimulus position 2106, the respective convergence angle θ is determined, when a convergence angle model is applied to process a respective eye image 2108 or the eye focal position 2110L or 2110R extracted from the respective eye image 2108.

In some embodiments, the convergence performance indicator 2112 includes a convergence error map 2116 of the two eyes measured with respect to the stimulus positions 2106. The computer device 140 may determine a plurality of convergence errors Δ of the two eyes corresponding to the plurality of stimulus positions, and generate the convergence error map 2116 of the two eyes with respect to the stimulus positions 2106. Further, in some embodiments, for each stimulus position 2106, the computer device 140 may determine a respective convergence angle θ (FIG. 20) based on a left eye focal position 2110L of a left eye, a right eye focal position 2110R of a right eye, and a gaze point 2004, determine a reference convergence angle η (FIG. 20) based on the left focal position 2110L, the right focal position 2110R, and the respective stimulus position 2106, and determine a respective convergence error Δ based on the respective convergence angle θ and the reference convergence angle η.

In some embodiments, the computer device 140 may select a subset of stimulus position 2106 based on the eye focal positions 2110 of the two eyes. Each eye may have a nominal position substantially in the middle of the eye when the eye looks forward. The greater a shift of the eye focal position 2110 from the nominal position, the closer the corresponding stimulus position 2106 to the user 120. In some embodiments, when the shift of the eye focal position 2110 from the nominal position increases, a larger number of visual stimuli 2104 are applied, allowing a higher density of convergence angles θ to be measured for a portion of the field of view located near the user 120.

In some embodiments, the computer device 140 may include a motion sensor 376 coupled to the HMD 312A, and obtain motion data captured by the motion sensor 376. For each of the plurality of stimulus positions 2106, the computer device 140 may determine an orientation 2118 of the HMD 312A based on the motion data, and adjust the convergence performance indicator 2112 for the two eyes based on the orientation 2118 of the HMD 312A. During the vision test, the user 120 may be guided to keep the orientation 2118 of the HMD by facing forward, so that the convergence angle θ may be properly scanned and map for a target portion of the field of view of the user 120. For example, a peripheral area (e.g., region 2008 in FIG. 20) of the user may have a larger convergence error, and the user 120 is used to varying the orientation 2118 to face the peripheral area for the purposes of getting an accurate convergence angle θ. Tracking the orientation 2118 of the HMD 312A may allow the peripheral area having impaired convergence performance to be property identified.

In some embodiments, the convergence performance indicator 2112 may include a convergence angle range 2120 identifying a range of convergence angles θ measured in response to the plurality of visual stimuli 2104. The plurality of stimulus positions 2106 may be located in a targeted portion of the field of view of the user 120, and the convergence angle range 2120 may be associated with the target portion of the field of view.

In some embodiments, the convergence performance indicator 2112 may include a convergence deficiency area 2122 (e.g., region 2008). The convergence angles θ corresponding to the visual stimuli 2104 displayed in the convergence deficiency area 2122 may have convergence errors Δ greater than an error tolerance.

In some embodiments, for a first stimulus position (e.g., 1208A in FIG. 13), the computer device 140 may identify a respective set of one or more eye focal positions 2110L or 2110R corresponding to a respect set of one or more gaze points 2004 (e.g., P3, P4, P5) that satisfy a predefined response criterion. Further, in some embodiments, the predefined response criterion requires that, for the first stimulus position, the respective set of one or more gaze points 2004 are located within a respective physical range 1302 (FIG. 13) surrounding the respective stimulus position 2106. In some embodiments, the respective physical range 1302 may correspond to a tolerance range r defined based on an error tolerance for a convergence error Δ (e.g., 0-10°), and the gaze points 2004 located in the respective physical range 1302 corresponding to the tolerance range r of the stimulus location 2106A may be determined to satisfy the predefined response criterion.

In some embodiments, after a visual stimulus 2104 is displayed at the first stimulus position (e.g., 1208A in FIG. 13), a sequence of intermediate gaze points (e.g., P1-P11 in FIG. 13) may be extracted from the eye images 2108, e.g., moving from external to the physical range 1302 into the physical ranges 1302 and stabilizing at the respective set of one or more gaze points (e.g., P9-P11). The respective stimulus position 2106 (e.g., position 1208A in FIG. 13) corresponds to a stimulus time, and each gaze point 2004 corresponds to a focal time measured with respect to the stimulus time. The focal time may indicate a time duration taken by the respective gaze point 2004 to stabilize in response to the respective visual stimulus 2104. The computer device 140 may determine an average gaze point 1304 (FIG. 13) of the respective set of one or more gaze points, and the average gaze point 1304 may be used to represent the gaze point 2004 used to assess convergence performance of the eyes (e.g., determining the convergence error Δ or the focal time). Referring to FIG. 13, a position offset may be determined based on the average gaze point 1304 and the stimulus position 1208A. Referring to FIG. 20, the position offset may be determined based on the gaze point 2004 and the object location 2006. A respective convergence error Δ may be determined based on the respective stimulus position and the average gaze point.

In some embodiments, the computer device 140 may execute a media play application 2124 for playing media content. Media data associated with the media content are adjusted based on the convergence performance indicator 2112. The media content may be displayed based on the adjusted media data. For example, a convergence deficiency area 2122 (e.g., region 2008) may be identified, and the media data are adjusted to compensate convergence deficiency in the area 2122. In some embodiments, a refresh rate or a display area is adjusted based on the convergence performance indicator 2112.

Some implementations of this application are directed to implementing a vision test for convergence performance of a user's eyes. The vision test process 1200 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may display a sequence of visual stimuli 2104 on the user interface 2102, and obtain a sequence of eye images 2108 of two eyes of a user 120 associated with the computer device 140 (e.g., a user 120 wearing a headset device 140D). A sequence of eye focal positions 2110 may be determined in the sequence of eye images 2108. The computer device 140 may generate a convergence angle map 2114 of the two eyes based on at least the sequence of eye focal positions 2110.

In some embodiments, the sequence of visual stimuli 2104 may correspond to a plurality of stimulus positions 2106 distributed in the 3D virtual environment. Further, in some embodiments, the computer device 140 may determine a plurality of convergence angles θ of the two eyes corresponding to the plurality of stimulus positions 2106. Additionally, in some embodiments, for each stimulus position, the computer device 140 may determine a left eye focal position 2110L of a left eye and a right eye focal position 2110R of a right eye. A gaze point 2004 may be determined based on the left focal position 2110L and the right focal position 2110R, and a respective convergence angle θ may be further determined based on the left eye focal position 2110L, the right eye focal position 2110R, and the gaze point 2004.

### Assessment of Visual Hallucination Conditions

Some implementations of this application include a VR-based computer system 300 configured to simulate and test responses to visual hallucinations for neurological assessment. The computer system 300 may utilize a high-resolution VR headset that may be equipped with eye-tracking sensors (e.g., eye-tracking cameras 366 in FIG. 3) and a visual assessment application to create controlled, realistic visual hallucinations within an immersive virtual environment. Users wear the VR headset and engage in a series of tasks and scenarios where visual hallucinations are introduced in a controlled manner. The eye-tracking camera 366 may monitor the user's gaze direction, fixation points, and eye movements, and the visual assessment application may analyze these responses to assess the user's cognitive and neurological reactions to the hallucinations. The VR-based computer system 300 may diagnose and monitor neurological disorders that manifest with visual hallucinations, such as schizophrenia, Parkinson's disease, and certain types of dementia.

In some embodiments, the VR-based computer system 300 may incorporate a range of scenarios where users encounter different types of visual hallucinations, such as floating objects, shifting patterns, and unreal visual distortions. These scenarios may be crafted to be engaging and non-threatening, ensuring that users can interact with the virtual environment in which the hallucinations are visually presented. A user application 324 (e.g., visual assessment application 328 in FIG. 3) may process the data in real time, and evaluate parameters such as reaction time, gaze stability, and the user's ability to distinguish between real and hallucinatory stimuli. Results may be compiled into a report that provides insights into the user's neurological health, highlighting any atypical responses that could indicate underlying conditions. As such, the computer system 300 may offer a dynamic, precise, and non-invasive approach for assessing the impact of visual hallucinations on cognitive function, representing a significant advancement over traditional diagnostic methods.

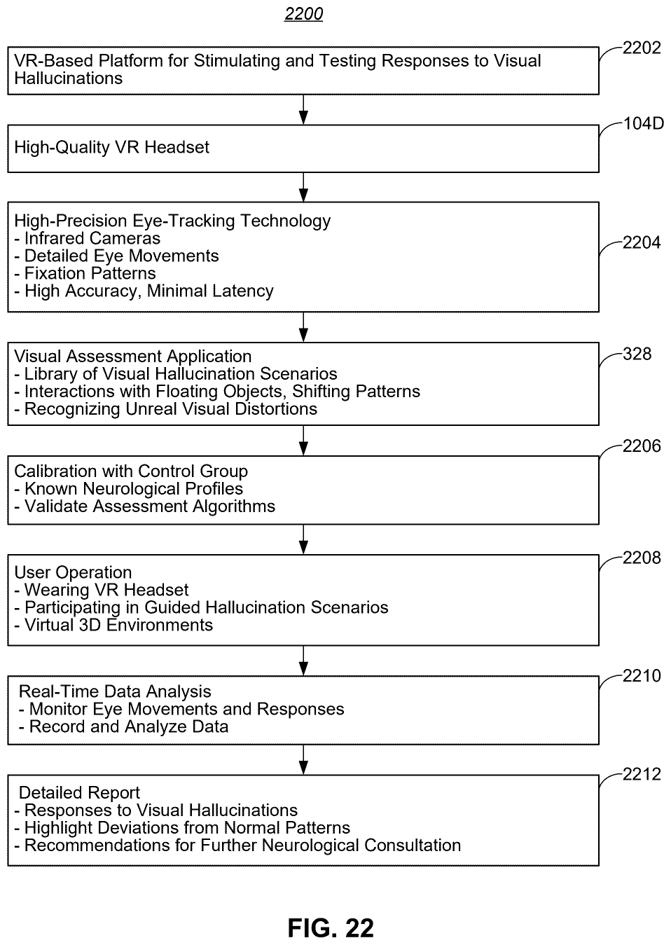

FIG. 22 is a flow diagram of an example vision test process 2200 for assessing a hallucination condition of a user's visual system, in accordance with some embodiments. The VR-based computer system 300 may be configured to enable a VR-based platform 2202 for simulating and testing responses to visual hallucinations. The computer system 300 may include a VR headset 104D that includes an eye-tracking camera 366 (FIG. 3). The eye-tracking technology may include an infrared camera (e.g., camera 366) configured to capture (operation 2204) eye movements and fixation patterns with high accuracy and minimal latency. In some embodiments, when a visual assessment application 328 is executed, a library of visual hallucination scenarios may be used to test different aspects of cognitive and neurological responses. These scenarios include interactions with floating objects, navigating through environments with shifting patterns, and recognizing unreal visual distortions within the virtual world.

In some embodiments, when hardware components and software modules may be integrated to form the VR-based platform 2202, the VR-based computer system 300 may be calibrated (operation 2206) using a control group of individuals with known neurological profiles to establish baseline performance metrics and validate the accuracy of the assessment algorithms. In some embodiments, users may operate (operation 2208) the system by wearing the VR headset and participating in the guided hallucination scenarios within the virtual environments. The eye-tracking camera 366 may monitor their eye movements and responses to the hallucinations. Image or video data recorded by the camera 366 may be analyzed (operation 2210) in real time by the software modules (e.g., visual assessment application 328, data processing module 330 in FIG. 3). In some implementations, the user may receive a report 2212 outlining their responses to the visual hallucinations, highlighting any deviations from typical patterns, and providing recommendations for further neurological consultation. By these means, the computer system 140 may offer a precise, non-invasive, and user-friendly method for assessing the neurological impact of visual hallucinations, representing a significant advancement over traditional testing techniques and providing substantial benefits for both clinical and research applications.

FIG. 23 is a flow diagram of an example vision test process 2300 for assessing a hallucination condition of a user's visual system in a 3D virtual environment, in accordance with some embodiments. The vision test process 2300 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2302 corresponding to a 3D virtual environment. The computer device 140 may display a sequence of visual hallucination patterns 2304. While displaying the visual hallucination patterns 2304, the computer device 140 may obtain a stream of sensor data from the one or more sensors 360 (FIG. 3). A plurality of user responses 2306 to the sequence of visual hallucination patterns 2304 may be determined based on the stream of sensor data. The computer device may determine a type 2308T and a severity level 2308S of a first visual hallucination condition 2308 of a user 120 associated with the computer device 140.

In some embodiments, the computer device 140 may extract a plurality of response feature vectors 2310 from the plurality of user responses 2306 and apply a hallucination diagnosis model 2312 to process the plurality of response feature vectors 2310 and generate an output vector 2314. Further, in some embodiments, the output vector 2314 may include a plurality of output elements (e.g., P1, P2, . . . , PN) each of which represents a respective severity level of a respective one of a plurality of known hallucination conditions 2316. For example, an output element P1 represents a severity level of a first known hallucination condition 2316A. Additionally, in some embodiments, the computer device 140 may identify a first output element greater than a threshold severity and determine that the first output element corresponds to the type 2308T of the first visual hallucination condition 2308, which corresponds to one of the plurality of known hallucination conditions 2316.

Alternatively, in some embodiments, the hallucination diagnosis model 2312 may include a classifier neural network, and the output vector 2314 may include a plurality of output elements (e.g., P1-PN) each of which represents a probability of having a respective one of a plurality of known hallucination conditions 2316. Further, in some embodiments, the computer device 140 may identify a first output element (e.g., P2) having the greatest value among the plurality of output elements, and determine that the first output element corresponds to the type 2308T of the first visual hallucination condition 2308. Additionally, in some embodiments, the computer device 140 may identify two or more output elements (e.g., P1 and P2) that have the greatest values among the plurality of output elements (e.g., P1-PN) and are greater than a threshold probability. The two or more output elements (e.g., P1 and P2) include the first output element (e.g., P2). The computer device 140 may determine that the first output element corresponds to the type 2308T of the first visual hallucination condition 2308. For example, the first visual hallucination condition 2308 of the user 120 corresponds to a known hallucination condition 2316B.

In some embodiments, the user 120 may have more than one visual hallucination conditions. The computer device 140 may determine a type 2318T and a severity level 2318S of each remainder visual hallucination condition 2318 distinct from the first visual hallucination condition 2308.

In some embodiments, each hallucination pattern 2304 of the sequence of visual hallucination patterns may correspond to a respective type and a respective severity level of a respective known hallucination condition 2316. The respective type and the respective severity level of the respective known hallucination condition 2316 may be processed by the hallucination diagnosis model 2312 jointly with the plurality of response feature vectors 2310.

In some embodiments, the sequence of visual hallucination patterns 2304 includes an ordered sequence of known hallucination patterns corresponding to a set of known hallucination conditions 2316, and each known hallucination condition 2316 corresponds to a subset of a collection of respective known hallucination patterns 2304. Further, in some embodiments, for each known hallucination condition 2316, the subset of respective known hallucination patterns may be arranged according to severity levels of the respective known hallucination condition 2316 to the respective known hallucination patterns 2304 correspond.

In some embodiments, the plurality of user responses 2306 may include a user input 2306A captured by a subset of one or more first sensors of the computer device 140, and the one or more first sensors may include a forward facing camera 378 (FIG. 3) for detecting a hand gesture, a microphone 380 (FIG. 3) for collecting an audio response, or a controller 390 (FIG. 3) for receiving a user physical force.

In some embodiments, the user responses 2306 may include a spontaneous user response 2306S monitored by one or more second sensors of the computer device 140. The one or more second sensors include one or more of: an eye tracking camera 366, a heart rate sensor, a body temperature sensor, a blood oxygen level, a Galvanic skin response sensor, a hand gesture camera (e.g., camera 378), a body gesture camera (e.g., camera 378), a microphone 380, a motion sensor 376, and a set of one or more brain activity electrodes 362. In some embodiments, the eye tracking camera 366 may monitor gaze point, pupil size, and saccadic movements (quick, simultaneous movements of both eyes in the same direction). The spontaneous user response 2306S may be automatically determined based on image data captured by the eye tracking camera 366. More specifically, in some embodiments, the image data captured by the eye tracking camera 366 may be processed (e.g., by a machine learning model 350 in FIGS. 3 and 4) to determine a focal point of the user's eyes, a pupil size variation, a reaction time, and a consistency level across a plurality of vision tests.

More specifically, in some embodiments, the stream of sensor data may include a stream of image data captured by an eye-tracking camera 366, and each respective visual hallucination pattern 2304 may correspond to a subset of image data indicating a user's spontaneous response 2306S to the respective visual hallucination pattern 2304. Further, in some embodiments, the computer device 140 may extract eye position 2320, pupil dilation information 2322, and retinal responses 2324 from the stream of image data and determine a focus level 2326 of the user 120 associated with the computer device 140.

Some implementations of this application are directed to implementing a vision test for assessing hallucination conditions of a user's eyes. The vision test process 1200 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may display a sequence of visual hallucination patterns 2304 and obtain a stream of sensor data from the one or more sensors 360 (FIG. 3). The computer device 140 may extract a plurality of spontaneous response feature vectors 2310 from the sensor data, and apply a hallucination diagnosis model 2312 to process at least the plurality of spontaneous response feature vectors and generate an output vector. A type and a severity level of a first visual hallucination condition 2308 may be determined for a user 120 associated with the computer device 140.

### Assessment of Selective Attention Capabilities

Some implementations of this application include a VR-based computer system 300 configured to evaluate selective attention capabilities in vision by using attention-demanding stimuli within a virtual environment. The computer system 300 may utilize a high-resolution VR headset that may be equipped with eye-tracking sensors (e.g., eye-tracking cameras 366 in FIG. 3) and a visual assessment application to create complex, engaging visual stimuli that require focused attention. Users wear the VR headset and engage in a series of tasks that involve identifying, tracking, and responding to specific stimuli while ignoring distracting elements. The eye-tracking camera 366 may monitor the user's gaze direction, fixation duration, and saccadic movements, while the software analyzes these responses to assess the user's selective attention capabilities.

In some embodiments, the VR-based computer system 300 may incorporate a range of tasks designed to challenge selective attention, such as detecting target objects among distractors, following specific moving targets in a busy environment, and responding to changing visual cues while maintaining focus on a primary task. These scenarios are applied to simulate real-world situations that demand high levels of selective attention, such as driving in heavy traffic or reading in a noisy environment. A user application 324 (e.g., visual assessment application 328 in FIG. 3) may process the data in real time, and evaluate parameters such as reaction time, accuracy, and the ability to maintain focus amidst distractions. Results may be compiled into a report that provides insights into the user's selective attention performance, identifying any deficiencies that could indicate conditions such as ADHD, visual processing disorders, or age-related cognitive decline. As such, the computer system 300 may offer a dynamic, engaging, and precise approach for assessing selective attention in a controlled virtual environment.

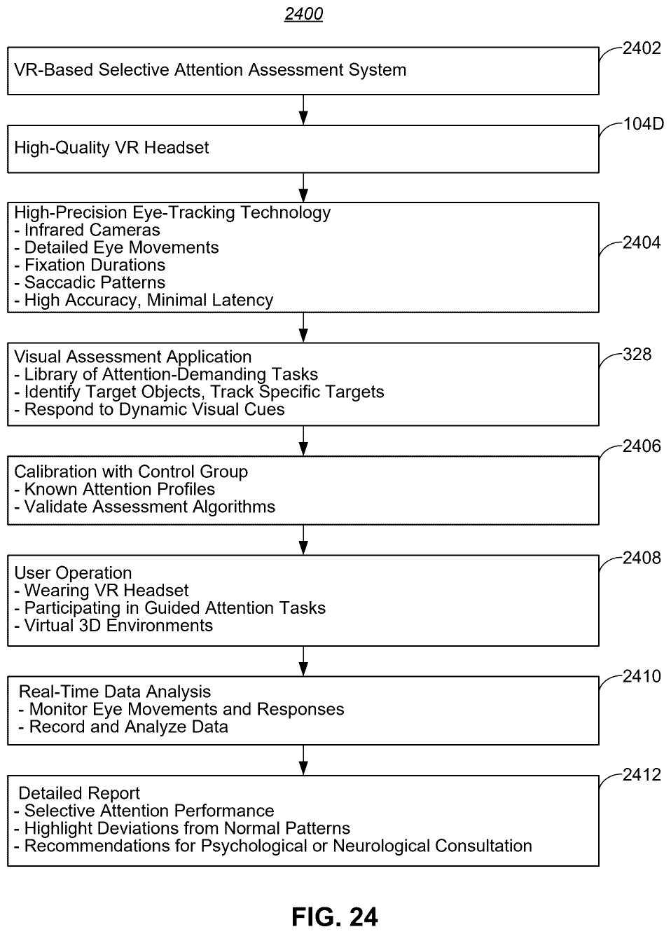

FIG. 24 is a flow diagram of an example vision test process 2400 for determining visual processing speed and accuracy of a user's visual system, in accordance with some embodiments. The VR-based computer system 300 may be configured to enable a VR-based selective attention assessment system 2402. The computer system 300 may include a VR headset 104D that includes an eye-tracking camera 366 (FIG. 3). The eye-tracking technology may include an infrared camera (e.g., camera 366) configured to capture (operation 2404) eye movements, fixation durations, and saccadic patterns with high accuracy and minimal latency. In some embodiments, when a visual assessment application 328 is executed, a library of attention-demanding tasks may be used to test different aspects of selective attention. These tasks include scenarios where users must identify target objects among numerous distractors, track specific targets moving within a complex visual field, and respond to visual cues that change dynamically while ignoring irrelevant stimuli.

In some embodiments, when hardware components and software modules may be integrated to form the VR-based selective attention assessment system 2402, the VR-based computer system 300 may be calibrated (operation 2406) using a control group of individuals with known attention profiles to establish baseline performance metrics and validate the accuracy of the assessment algorithms. In some embodiments, users may operate (operation 2408) the system 2402 by wearing the VR headset and participating in the guided attention tasks within the virtual environments. The eye-tracking camera 366 may monitor their eye movements and responses to the attention-demanding stimuli. Image or video data recorded by the camera 366 may be analyzed (operation 2410) in real time by the software modules (e.g., visual assessment application 328, data processing module 330 in FIG. 3). In some implementations, the user may receive a report 2412 outlining their selective attention performance, highlighting any deviations from normal patterns, and providing recommendations for further psychological or neurological consultation. By these means, the computer system 140 may offer a precise, non-invasive, and user-friendly method for assessing selective attention capabilities, representing a significant advancement over traditional testing techniques and providing substantial benefits for both clinical and research applications.

FIG. 25A is a flow diagram of an example vision test process 2500 for assessing a user's visual attention in a 3D virtual environment, in accordance with some embodiments. The vision test process 2500 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2502 corresponding to a 3D virtual environment. In some embodiments, the computer device 140 may display a plurality of visual stimuli 2504 concurrently in a 3D virtual environment, and each visual stimulus 2504 may be displayed at a position in the 3D virtual environment according to a display scheme 2506A or 2506B. The computer device 140 may obtain a stream of sensor data measured by one or more sensors 360 (FIG. 3) and determine a plurality of sequential user responses 2508 to the plurality of visual stimuli 2504 based on the stream of sensor data. Based on the plurality of sequential user responses 2508, the computer device 140 may determine an attention indicator 2510 indicating an attention capability of the user 120 associated with the computer device 140 to different visual stimuli 2504.

In some embodiments, for each of a first subset of visual stimuli 2504A, in accordance with the respective display scheme 2508A, the respective visual stimulus 2504A may be displayed with a respective flickering frequency f_{K} and an active duty cycle DC and configured to fade away and emerge at a varying rate R_{V}. Further, in some embodiments, for each of a second subset of visual stimuli 2504B, in accordance with the respective display scheme 2508B, the respective visual stimulus 2504B is continuously displayed without flickering.

In some embodiments, each of the plurality of visual stimuli 2504 is surrounded by a local context 2512, and the local context 2512 is displayed with a context scheme configured to create a respective level of distraction to the respective visual stimuli 2504. In some embodiments, each visual stimulus 2504 may be displayed with a set of display parameters 2514 including a display size, a resolution, a contrast level, and a brightness level. In some embodiments, the computer device 140 may generate an instruction 2516 to guide the user to identify the plurality of visual stimuli 2504 according to a sequential order.

In some embodiments, each of the plurality of sequential user responses 2508 includes a respective temporal variation of: a left eye position, a right eye position, a gaze point, a pupil size, a saccadic movement level, and a head orientation.

In some embodiments, the plurality of visual stimuli 2504 include a sequential order of visual stimuli 2504, and each of the plurality of sequential user responses 2508 corresponds to a respective sensor 360. The plurality of visual stimuli 2504 may include an ordered sequence of user response segments 2518 each of which corresponding to a respective visual stimulus 2504. Further, in some embodiments, for each sequential user response 2508, the computer device 140 may extract a plurality of user response feature vectors 2520 from the ordered sequence of user response segments 2518. Additionally, in some embodiments, each user response segment 2518 of a first sequential response may include a respective number of sensor data samples. For a subset set of user response segments of the first sequential response, the computer device may convert the respective number of sensor data samples to a predefined number of sensor data samples (e.g., by data sample interpolation or by sampling the respective number of sensor data samples). The predefined number of sensor data samples may be processed, e.g., by a feature extraction model 2522, which may extract a respective user response feature vector 2520 from the respective user response segment 2518.

In some embodiments, the computer device 140 may determine the attention indicator 2510 by applying an attention tracking model to process the plurality of sequential user responses 2508 and generate an output vector 2524 corresponding to the attention indicator 2510. Further, in some embodiments, the attention tracking model includes a feature extraction model 2522 and a feature tracking model 2526. In some embodiments, for each sequential user response 2508, the computer device 140 may extract a plurality of user response feature vectors 2520 corresponding to the respective sequential user response 2508. For each visual stimulus 2504, the computer device 140 may apply a feature extraction model 2528 and generate a stimulus feature vector 2530 based on characteristics of the respective visual stimulus 2504 and an associated local context 2512. The plurality of user response feature vectors 2520 of the plurality of sequential user responses 2508 and the stimulus feature vectors 2530 of the plurality of visual stimuli 2504 may be organized in a predefined data structure, forming an input feature structure 2532. The feature tracking model 2526 may be applied to process the input feature structure and generate the output vector 2524.

Additionally, in some embodiments, the output vector 2524 includes a plurality of output elements, and each output element may correspond to an attention related performance level 2534 selected from: a fixation duration, a display size limit, a flickering rate limit, and a distraction susceptibility level.

FIG. 25B is a flow diagram of another example vision test process 2550 for assessing a user's visual attention in a 3D virtual environment, in accordance with some embodiments. The vision test process 2550 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may display a plurality of visual stimuli 2504 concurrently in a 3D virtual environment. While displaying the visual stimuli 2504, the computer device 140 may obtain infrared video data 2560 recorded by the infrared eye tracking camera 366, and determine a plurality of sequential user responses 2508 to the plurality of visual stimuli 2504 based on the infrared video data 2560. The computer device 140 may determine a severity level 2552S and a type 2552T of an attention deficiency condition 2552 for the user 120 associated with the computer device 140 based on the plurality of sequential user responses 2508.

In some embodiments, the computer device may apply an attention tracking model 2554 to process the plurality of sequential user responses 2508 and generate an output vector 2556 including a plurality of output elements. The plurality of output elements may include the severity level 2552S and the type 2552T of the attention deficiency condition 2552 and one or more of: a deviation from a normal attention profile and a recommendation for further consultation.

### Assessment of Spatial Awareness and Balance

Some implementations of this application include a VR-based computer system 300 configured to assess the impact of head and body movement on spatial awareness and balance. The computer system 300 may utilize a VR headset integrated with motion tracking technology and a visual assessment application configured for generating immersive virtual environments. Users may wear the VR headset and engage in a series of tasks that involve dynamic head and body movements within the virtual space. The motion tracking sensors may continuously monitor the user's head and body movements, balance, and spatial orientation, and the visual assessment application may analyze these responses to assess the users' spatial awareness and balance capabilities under varying movement conditions.

In some embodiments, the VR-based computer system 300 may incorporate a range of interactive tasks, such as navigating through obstacle courses, maintaining balance on virtual narrow target paths, and responding to spatial cues while performing head and body movements. These tasks may be applied to simulate real-world scenarios that require precise spatial awareness and balance, such as walking on uneven terrain or adjusting posture while moving. A user application 324 (e.g., visual assessment application 328 in FIG. 3) may process the data in real time, and evaluate parameters such as movement accuracy, reaction time, and balance stability. Results may be compiled into a report that provides insights into the user's spatial awareness and balance performance, identifying any deficiencies that could indicate conditions such as vestibular disorders, balance impairments, or proprioceptive dysfunction. As such, the computer system 300 may offer a dynamic, engaging, and precise approach for assessing the impact of head and body movement on spatial awareness and balance in a controlled virtual environment.

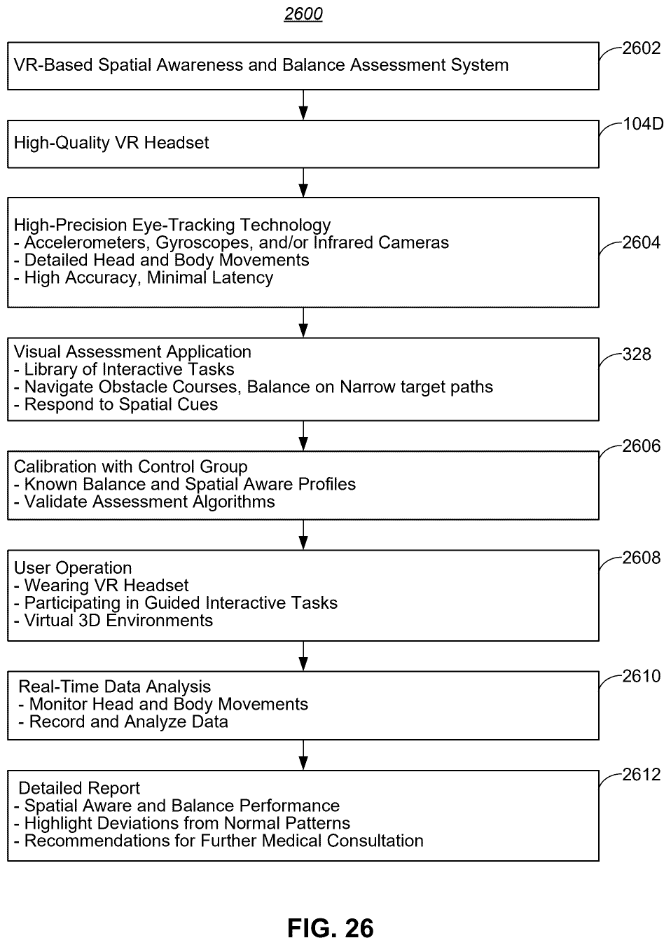

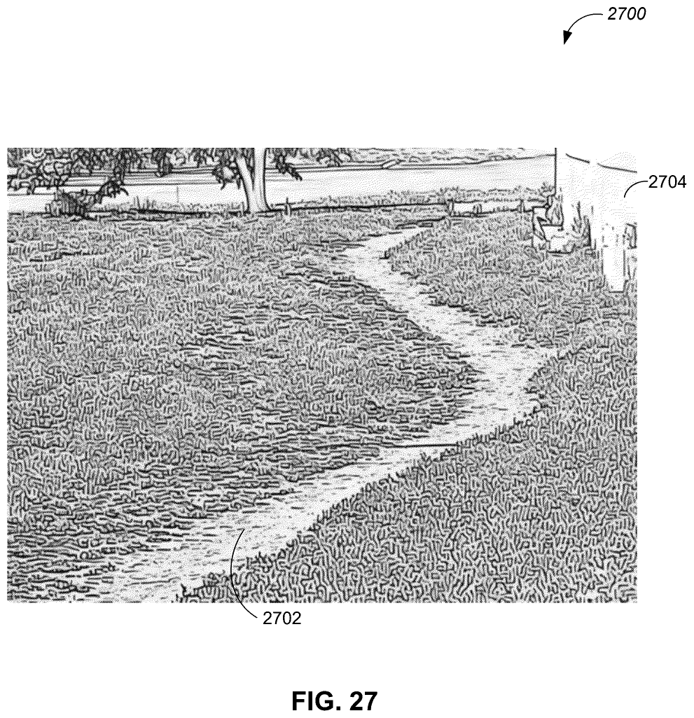

FIG. 26 is a flow diagram of an example vision test process 2600 for assessing spatial awareness and balance associated with a user's visual system, in accordance with some embodiments. The VR-based computer system 300 may be configured to enable a VR-based spatial awareness and balance assessment system 2602. The computer system 300 may include a VR headset 104D that includes motion tracking sensors. The motion tracking sensors may include accelerometers, gyroscopes, or infrared cameras configured to capture (operation 2604) head and body movements with high accuracy and minimal latency. In some embodiments, when a visual assessment application 328 is executed, a library of interactive tasks may be used to test different aspects of spatial awareness and balance. These tasks may include scenarios where users must navigate through virtual obstacle courses, maintain balance on simulated narrow target paths, and respond to spatial cues while performing coordinated head and body movements.

In some embodiments, when hardware components and software modules may be integrated to form the VR-based spatial awareness and balance assessment system 2602, the VR-based computer system 300 may be calibrated (operation 2606) using a control group of individuals with known balance and spatial awareness profiles to establish baseline performance metrics and validate the accuracy of the assessment algorithms. In some embodiments, users may operate (operation 2608) the system 2602 by wearing the VR headset and participating in the guided interactive tasks within the virtual environments. The motion tracking sensors may monitor a user's head and body movements, balance, and spatial orientation. Image or video data recorded by the camera 366 may be analyzed (operation 2610) in real time by the software modules (e.g., visual assessment application 328, data processing module 330 in FIG. 3). In some implementations, the user may receive a report 2612 outlining their spatial awareness and balance performance, highlighting any deviations from normal patterns, and providing recommendations for further medical consultation. By these means, the computer system 140 may offer a precise, non-invasive, and user-friendly method for assessing the impact of head and body movement on spatial awareness and balance, representing a significant advancement over traditional testing techniques and providing substantial benefits for both clinical and research applications.

FIG. 27 is a diagram of a 3D visual environment 2700 including a target path 2702, in accordance with some embodiments. The target path 2702 may include a narrow trail that winds from a nearby vantage point and stretches farther. The target path 2702 may curve and fade into the distance, eventually disappearing after turning around a corner of a building 2704. When the target path 2702 is displayed in the 3D visual environment 2700, a user 120 wearing a headset device 140 where the visual environment 2700 is rendered may be prompted to move along the target path 2702 according to an instructed manner (e.g., “increasing your speed,” “slowing down,” “jump”). As the user 120 moves along the target path 2702, a view rendered in the 3D visual environment 2700 may be updated with an extended portion of the target path 2702 (e.g., hidden behind the building 2704) emerging in the environment 2700.

FIG. 28 is a flow diagram of an example vision test process 2500 for assessing spatial awareness of a user's visual system in a 3D virtual environment, in accordance with some embodiments. The vision test process 2800 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2802 corresponding to a 3D virtual environment. The computer device 140 may display a destination 2804 (e.g., building 2704 in FIG. 27) and a target path 2806 (e.g., path 2702 in FIG. 27) leading to the destination 2804 in the 3D virtual environment. The target path 2806 may follow at least one direction. In some embodiments, the target path 2806 may have one or more turns heading to different directions. The computer device 140 may render a request 2808 (e.g., by way of a visual sign or an audio message) for a user 120 associated with the computer device 140 to follow the target path 2806 to reach the destination 2804. A stream of sensor data 2810 may be collected from the one or more motion sensors 2812, while the user 120 moves along the target path 2806. Based on the stream of sensor data 2810, the computer device 140 may determine a directionality indicator 2820 of the user's visual system, and the directionality indicator 2820 may quantitatively represent a capability of the user's visual system following the at least one direction.

In some embodiments, the one or more motion sensors 2812 of the computer device 140 includes an outward camera 378, a set of accelerometers 2814, and a set of a gyroscopes 2816. The accelerometers 2814 and gyroscopes 2816 may be included in the 6DOF position and motion sensors 376 (FIG. 3) of the computer device 140. In some embodiments, the computer device 140 may further include one or two controllers 390 (FIG. 3) configured to be held by the user's hands, and the one or more motion sensors 2812 may further include supplemental sensors 2818 located in the one or two controllers 390.

In some embodiments, the computer device 140 may reconstruct a user path 2822 based on the stream of sensor data 2810 and compare the user path 2822 with the target path 2806 to determine a path fitting level 2824. The directionality indicator 2820 is determined based on the path fitting level 2824. Further, in some embodiments, the user path 2822 includes a plurality of positions 2826. The computer device 140 may determine a speed 2828 of the user 120 associated with each of the plurality of positions 2826 based on the stream of sensor data 2810. The directionality indicator 2820 may be determined based on the path fitting level 2824 and the speeds 2828 of the user 120 at the plurality of positions 2826.

In some embodiments, the directionality indicator 2820 of the user's visual system is generated after the user reaches the destination 2804. Further, in some embodiments, the computer device 140 may extract a sensor feature vector 2830 based on a subset of sensor data corresponding to each of the one or more motion sensors 2812, and select a directionality analysis model 2832 from a plurality of predefined model options 2834 based on the destination 2804 and the target path 2806. The selected directionality analysis model 2832 may be applied to process the sensor feature vectors 2830 of the one or more motion sensors 2812 and generate an output vector 2836, and the directionality indicator 2820 may be determined based on the output vector 2836. Additionally, in some embodiments, the output vector 2836 may include a subset of respective elements corresponding to each of a plurality of directions 2840 (e.g., forward, backward, left, right, forward shifted to the left by 45 degrees), and the directionality indicator 2820 may include a plurality of directionality scores 2838 (e.g., Score 1, Score 2, . . . , Score N) corresponding to the plurality of directions 2840. The computer device 140 may generate the plurality of directionality scores 2838 based on the respective elements of the output vector corresponding to the plurality of directions 2840. In an example, the plurality of directions 2840 include eight directions (e.g., forward, backward, left, right, 45 degrees to the left from the front, 45 degrees to the right from the front, 45 degrees to the left from the rear, and 45 degrees to the right from the rear), and every two directions are separated by 45 degrees.

In some embodiments, the directionality indicator 2820 of the user's visual system may be generated concurrently while the user moves along the target path 2806. Further, in some embodiments, each sensor 2830 may have a sensor sampling frequency f_{S}, and the directionality indicator 2820 may be generated at a directionality assessment frequency f_{D} corresponding to a directionality time window. The directionality assessment frequency f_{D} is smaller than the sensor sampling frequency f_{S}. Additionally, in some embodiments, after the computer device 140 may determine a set of first directionality indicator samples, the computer device 140 may dynamically adjust at least the target path 2806 associated with the destination 2804 for one or more subsequent directionality time windows. More specifically, in some embodiments, the computer device 140 may dynamically adjust at least the target path 2806 by determining whether the set of first directionality indicator samples satisfies a directionality criterion and adjusting a difficulty level 2842 of the target path 2806 for the one or more subsequent directionality time windows.

In some embodiments, the computer device 140 may execute a sport training application 2844 configured to manage training of athletes. The visual assessment application 328 is coupled to the sport training application 2844 via an application programming interface (API) 2846, and the sport training application 2844 is executed within the visual assessment application 328 via the API 2846. The computer device 140 may feed the directionality indicator 2820 of the user's visual system to the sport training application via the API.

Some implementations of this application are directed to implementing a vision test for assessing hallucination conditions of a user's eyes. The vision test process 1200 may be implemented by a computer device 140 (e.g., a headset device 140D). The computer device 140 may further include one or more processors, memory storing instructions to be executed by the one or more processors, and a HMD 312A. The computer device 140 may execute a user application 324 (e.g., a visual assessment application 328) configured to enable the vision test and generate a VR user interface 2102 corresponding to a 3D virtual environment. The computer device 140 may execute a sport training application for athlete training display, and display a destination 2804 and a target path 2806 leading to the destination 2804 in the 3D virtual environment. The computer device 140 may obtain a stream of sensor data 2810 collected from the one or more motion sensors 2812 while the user 120 moves along the target path 2806. Based on the stream of sensor data 2810, the computer device 140 may determine a directionality indicator 2820 of the user's visual system quantitatively representing a direction managing capability of the user's visual system. In some embodiments, the sport training application 328 is applied to train basketball players. In some embodiments, the directionality indicator 2820 includes a plurality of first scores each of which corresponds to a respective one of a plurality of directions. Each first score indicates how well the user 120 may follow the respective one of the plurality of directions. In some embodiments, the directionality indicator includes a plurality of second scores each of which corresponds to a respective speed to respond to a change between respective two of a plurality of directions.

### Illustration of the Subject Technology As Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for implementing a vision test, comprising: at an electronic device having a head-mounted display (HMD), one or more processors, and memory: executing a visual assessment application, including displaying a user interface to create a 3D virtual environment; while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment; determining a sequence of 3D gaze positions of the eyes in the 3D virtual environment based on the sequence of eye images; and determining a visual processing performance factor for the user based on the sequence of stimulus positions and the sequence of 3D gaze positions.

Clause 2. The method of Clause 1, further comprising: adaptively determining one or more display parameters for displaying media content on the HMD based on the visual processing performance factor.

Clause 3. The method of Clause 1 or 2, determining the sequence of 3D gaze positions of the eyes further comprising: extracting a region of interest (ROI) image of the two eyes from each eye image to form a sequence of ROI images based on the sequence of eye images; and applying a focus tracking model to process the sequence of ROI images and generate an output vector including the sequence of 3D gaze positions.

Clause 4. The method of any of Clauses 1-3, determining the sequence of 3D gaze positions of the eyes further comprising, for each eye image captured at a respective time: generating a region of interest (ROI) image of the two eyes in the respective eye image; determining a respective 3D gaze position based on the ROI image.

Clause 5. The method of any of Clauses 1-4, determining the sequence of 3D gaze positions of the eyes further comprising, for each eye image captured at a respective time: identifying a left eye center and a right eye center; determining a left line of sight and a right line of sight; and determining a respective 3D gaze position as an intersection point of the left line of sight and the right line of sight.

Clause 6. The method of any of Clauses 1-5, wherein determining the visual processing performance factor further comprising: determining at least one of a visual processing speed and a visual processing accuracy.

Clause 7. The method of any of Clauses 1-6, further comprising: based on the visual processing performance factor, determining a visual processing deficiency condition.

Clause 8. The method of any of Clauses 1-7, wherein each pair of two successive visual stimuli of the sequence of visual stimuli has a respective stimulus position change, determining the visual processing performance factor further comprising: determining a relationship of the visual processing performance factor and the respective stimulus position change.

Clause 9. The method of any of Clauses 1-8, determining the visual processing performance factor further comprising: for each stimulus position, identifying a respective set of one or more 3D gaze positions that satisfy a predefined response criterion.

Clause 10. The method of Clause 9, wherein the predefined response criterion requires that for each stimulus position, the respective set of one or more 3D gaze positions are located within a respective physical range surrounding the respective stimulus position.

Clause 11. The method of Clause 9 or 10, wherein each stimulus position corresponds to a stimulus time, and each eye focal position corresponds to a focal time, wherein the visual processing performance factor is determined based on the stimulus time of each of a subset of stimuli and the focal time of each of the respective set of one or more 3D gaze positions corresponding to each stimulus position.

Clause 12. The method of Clause 11, determining the visual processing performance factor further comprising: for each stimulus position, identifying a respective response time corresponding to a first focal point having the earliest focal time among the respective set of one or more 3D gaze positions; and determining a visual processing speed based on one or more respective response times of a subset of one or more stimulus positions.

Clause 13. The method of Clause 11 or 12, determining the visual processing performance factor further comprising; for each stimulus position, determining an average eye focal position of the respective set of one or more 3D gaze positions, and determining a position offset based on the average eye focal position and the stimulus position; and determining a visual processing accuracy based on the position offsets of the stimulus positions.

Clause 14. The method of any of Clauses 1-13, further comprising: determining a sequence of pupil sizes of at least one eye based on the sequence of eye images; determining a focus level of the sequence of pupil sizes; and adjusting the visual processing performance factor based on the focus level.

Clause 15. The method of any of Clauses 1-14, wherein the sequence of visual stimuli includes a known stimulus that is displayed sequentially at the sequence of stimulus positions.

Clause 16. A method for implementing a vision test, comprising: at an electronic device having a HMD, one or more processors, and memory: executing a visual assessment application, including displaying a user interface to create a 3D virtual environment; while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment; applying a visual processing assessment model to receive the sequence of eye images and the sequence of stimulus positions as inputs and generate a visual processing performance factor for the user.

Clause 17. The method of Clause 16, wherein the sequence of eye images corresponds to a sequence of ROI images of eye regions, obtaining the sequence of eye images further comprising extracting each eye image from tracking images captured by an eye tracking camera.

Clause 18. The method of Clause 16 or 17, applying the visual processing assessment model further comprising: extracting an image feature from each eye image; and generating a model input feature by arranging the image features of the sequence of eye images and the sequence of stimulus positions according to a predefined input data structure, wherein the model input feature is fed into the visual processing assessment model.

Clause 19. A method for implementing a vision test, comprising: at an electronic device including a head-mounted display (HMD), one or more processors, and memory: establishing a communication link between the electronic device and a controller held by a user associated with the electronic device; executing a user application configured to enable the vision test; displaying a VR user interface based on a driver license issuing requirement to create a 3D virtual environment, the VR user interface including a moving traffic scene on which one or more visual stimuli are displayed; and driving one or more actuators of a controller in synchronization with displaying the VR user interface.

Clause 20. The method of Clause 19, further comprising: generating a user instruction to request a user to provide a user input via the controller in response to displaying the one or more visual stimuli.

Clause 21. The method of Clause 19 or 20, wherein the one or more actuators of a controller are configured to vibrate the controller with a vibration scale.

Clause 22. The method of Clause 21, further comprising: identifying a presumed speed of a virtual vehicle associated with the vison test; setting the vibration scale based on the presumed speed; and setting a scene changing rate based on the presumed speed; wherein during an extended duration of time, the moving traffic scene is dynamically generated based on the scene changing rate, and the controller is dynamically vibrated based on the vibration scale.

Clause 23. The method of Clause 21 or 22, further comprising adding a virtual road bump effect to the moving traffic scene, including: setting a road bumpiness level; setting the vibration scale based on the road bumpiness level; and setting a scene changing rate based on the road bumpiness level; wherein during a shortened duration of time, the moving traffic scene is generated based on the scene changing rate, and the controller is vibrated based on the vibration scale.

Clause 24. The method of any of Clauses 19-23, wherein the one or more actuators of a controller are configured to heat the controller held by the user.

Clause 25. The method of any of Clauses 19-24, wherein the one or more actuators of the controller are driven to send a reminder to the user indicating a traffic situation.

Clause 26. The method of Clause 25, further comprising: obtaining a sequence of eye images; while displaying the VR user interface, tracking a focus level of the user based on the sequence of eye images, wherein the one or more actuators of the controller are driven in accordance with a determination that the focus level of the user drops below a predefined focus level.

Clause 27. The method of Clause 26, wherein tracking the focus level of the user further comprises: determining a pupil size for each of the sequence of eye images, wherein the focus level is tracked based on the pupil size of each eye image.

Clause 28. The method of any of Clauses 19-27, further comprising: based on the driver license issuing requirement, determining a disturbance associated with the moving traffic scene; based on the disturbance, playing an audio message in synchronization with driving the one or more actuators of the controller and displaying the VR user interface.

Clause 29. The method of any of Clauses 19-28, wherein the driver license issuing requirement includes a predefined duration of time, the method further comprising: determining that the moving traffic scene has been displayed for a predefined duration of time, wherein in accordance with a determination that the moving traffic scene has been displayed for the predefined duration of time, the one or more actuators of the controller are driven to remind the user of the predefined duration of time.

Clause 30. The method of Clause 29, further comprising selecting the predefined duration of time based on a type of the moving traffic scene.

Clause 31. The method of any of Clauses 19-30, wherein: the one or more visual stimuli include a plurality of visual stimuli, and the driver license issuing requirement includes a respective duration of time for each of the plurality of stimuli; and for each of the plurality of stimuli, the one or more actuators of the controller are driven, in accordance with a determination that a length of displaying the moving traffic scene has reached the respective duration of time and that no user response to the respective stimulus has been received.

Clause 32. A method for implementing a vision test, comprising: at an electronic device including a head-mounted display (HMD), one or more processors, and memory: establishing a communication link between the electronic device and a controller held by a user associated with the electronic device; executing a media play application to enable a 3D user interface; displaying media content on the 3D user interface; obtaining media metadata associated with the media content; generating a controller instruction based on the media metadata; and applying the controller instruction to drive one or more actuators of a controller in synchronization with the media content.

Clause 33. The method of Clause 32, wherein the controller instruction includes a vibration scale, and the one or more actuators of the controller are configured to vibrate the controller with the vibration scale.

Clause 34. The method of Clause 32 or 33, wherein the one or more actuators of a controller are configured to heat the controller held by the user in response to the controller instruction.

Clause 35. A method of implementing a vision test: at an electronic device including a HMD and an infrared camera: executing a visual assessment application, including displaying a user interface to create a 3D virtual environment; displaying a body of text on the user interface for an extended duration of time; obtaining a sequence of eye images, each eye image including a respective infrared image of a region of interest (ROI) corresponding to at least one eye; based on the sequence of eye images, determining an eye endurance level of the at least one eye of a user associated with the electronic device.

Clause 36. The method of Clause 35, further comprising: selecting a predefined brightness level and a predefined font size, wherein the body of text is displayed with the predefined brightness level and the predefined font size.

Clause 37. The method of Clause 35 or 36, further comprising: directing the infrared camera towards the at least one eye; capturing by the infrared camera a sequence of camera images including the ROI corresponding to the at least one eye; and for each camera image, cropping a respective one of the sequence of camera images based on the ROI to generate the respective eye image.

Clause 38. The method of any of Clauses 35-37, determining the eye endurance level further comprising: applying an eye endurance model to process the sequence of eye images and generate a model output including the eye endurance level.

Clause 39. The method of Clause 38, wherein the model output includes a diagnosis indicator identifying a dry eye severity level associated with the eye endurance level.

Clause 40. The method of Clause 38 or 39, wherein the eye endurance model includes a feature extraction model and an endurance assessment model, applying the eye endurance model further comprising: applying the feature extraction model to extract a respective eye feature vector from each of the sequence of eye images; applying the endurance assessment model to process respective eye feature vectors of the sequence of eye images and generate the model output.

Clause 41. The method of any of Clauses 35-40, wherein the eye endurance level is determined with respect to a predefined temporal length that is greater than the extended duration of time.

Clause 42. The method of Clause 41, further comprising: receiving an eye endurance model from a server communicatively coupled to the electronic device; and at the server, training the eye endurance model using training data including a sequence of eye images and a ground truth eye endurance level corresponding to the predefined temporal length.

Clause 43. The method of any of Clauses 35-42, further comprising: executing a media play application to display multimedia content on the electronic device; and controlling execution of the media play application based on the eye endurance level.

Clause 44. The method of any of Clauses 35-43, determining the eye endurance level further comprising: detecting one or more eye blinking events and one or more eye blinking times; determining a sequence of eye lid positions, each eye lid position corresponding to a respective eye image of the sequence of eye images; and determining a sequence of pupil sizes, each pupil size corresponding to a respective eye image of the sequence of eye images; wherein the eye endurance level is determined based on the one or more eye blinking times, the sequence of eye lid positions, and the sequence of pupil sizes.

Clause 45. The method of Clause 44, determining the eye endurance level further comprising: tracking the on the one or more eye blinking times, the sequence of eye lid positions, and the sequence of pupil sizes with reference to a start time of displaying the body of text.

Clause 46. The method of Clause 44 or 45, further comprising applying an eye endurance model to process the one or more eye blinking times, the sequence of eye lid positions, and the sequence of pupil sizes and determine the model output including the eye endurance level.

Clause 47. The method of any of Clauses 35-46, determining the eye endurance level further comprising: extracting a sclera feature from each of the sequence of eye images; applying an eye endurance model to determine an eye dryness feature based on the respective sclera features of the sequence of eye images, the eye endurance level is determined based on respective sclera features.

Clause 48. A method of implementing a vision test: at an electronic device including a HMD and an infrared camera: displaying a visual pattern on the user interface for an extended duration of time; obtaining a sequence of eye images from an eye-tracking camera, each eye diagram including a sclera area; and applying an eye endurance model to process the sequence of eye images and generate a model output including a dry eye indicator associated with a dry eye condition.

Clause 49. The method of Clause 48, wherein the dry eye indicator indicates whether there is a dry eye condition and includes a dry eye severity level.

Clause 50. The method of Clause 48 or 49, further comprising selecting a predefined brightness level, wherein the visual pattern is displayed with the predefined brightness level.

Clause 51. The method of any of Clauses 48-50, wherein the visual pattern includes a body of text.

Clause 52. The method of any of Clauses 48-51, wherein the visual pattern includes a sequence of image frames configured to show accelerated motion.

Clause 53. The method of any of Clauses 48-52, further comprising cropping the sequence of eye images to extract the sclera area from each eye image and generating a sequence of sclera images, and the eye endurance model is applied to generate the model output based on the sequence of sclera images.

Clause 54. A method of implementing a vision test, comprising: at an electronic device having a head-mounted display (HMD), one or more processors, and memory: executing a visual assessment application, including displaying a user interface to create a 3D virtual environment; displaying a sequence of visual stimuli on the user interface, wherein the sequence of visual stimuli corresponds to a plurality of stimulus positions distributed in the 3D virtual environment; obtaining a sequence of eye images of two eyes of a user associated with the electronic device; determining a sequence of eye focal positions of the eyes in the sequence of eye images; and determining a convergence performance indicator for the two eyes of the user based on at least the sequence of eye focal positions.

Clause 55. The method of Clause 54, wherein the convergence performance indicator includes a map of convergence angles of the two eyes measured with respect to the plurality of stimulus positions, determining the convergence performance indicator further comprising: determining a plurality of convergence angles of the two eyes corresponding to the plurality of stimulus positions; and generating the map of convergence angles of the two eyes with respect to the plurality of stimulus positions.

Clause 56. The method of Clause 55, determining the plurality of convergence angles of the two eyes further comprising, for each stimulus position: determining a respective convergence angle based on a left eye focal position of a left eye, a right eye focal position of a right eye, and a gaze point.

Clause 57. The method of Clause 56, determining the plurality of convergence angles of the two eyes further comprising, for each stimulus position, determining the left eye focal position and the right eye focal position; and determining a gaze point based on the left focal position and the right focal position.

Clause 58. The method of Clause 54 or 55, wherein the convergence performance indicator includes a convergence error map of the two eyes measured with respect to the plurality of stimulus positions, determining the convergence performance indicator further comprising: determining a plurality of convergence errors of the two eyes corresponding to the plurality of stimulus positions; generating the map of convergence errors of the two eyes with respect to the plurality of stimulus positions.

Clause 59. The method of Clause 58, determining the plurality of convergence errors of the two eyes further comprising, for each stimulus position: determining a respective convergence angle based on a left eye focal position of a left eye, a right eye focal position of a right eye, and a gaze point; determining a reference convergence angle based on the left focal position, the right focal position, and the respective stimulus position; and determining a respective convergence error based on the respective convergence angle and the reference convergence angle.

Clause 60. The method of any of Clauses 54-59, further comprises setting a subset of stimulus position based on the eye focal positions of the two eyes.

Clause 61. The method of any of Clauses 54-60, wherein the electronic device includes a motion sensor coupled to the HMD, further comprising: obtaining motion data captured by the motion sensor; and for each of the plurality of stimulus positions: determining an orientation of the HMD based on the motion data; and adjusting the convergence performance indicator for the two eyes based on the orientation of the HMD.

Clause 62. The method of any of Clauses 54-61, wherein the convergence performance indicator includes a convergence angle range.

Clause 63. The method of any of Clauses 54-62, wherein the convergence performance indicator includes a convergence deficiency area.

Clause 64. The method of any of Clauses 54-63, further comprising, for a first stimulus position, identifying a respective set of one or more eye focal positions corresponding to a respect set of one or more gaze points that satisfy a predefined response criterion.

Clause 65. The method of Clause 64, wherein the predefined response criterion requires that for the first stimulus position, the respective set of one or more gaze points are located within a respective physical range surrounding the respective stimulus position.

Clause 66. The method of Clause 65, determining the convergence performance indicator further comprising, for the first stimulus position: determining an average gaze point of the respective set of one or more gaze points; determining a position offset based on the average gaze point and the stimulus position; and determining a respective convergence error based on the respective stimulus position and the average gaze point.

Clause 67. The method of any of Clauses 54-66, further comprising: executing a media play application for playing media content; adjusting media data associated with the media content based on the convergence performance indicators; and displaying the media content based on the adjusted media data.

Clause 68. A method of implementing a vision test, comprising: at an electronic device having a head-mounted display (HMD), one or more processors, and memory: executing a visual assessment application, including displaying a user interface to create a 3D virtual environment; displaying a sequence of visual stimuli on the user interface; obtaining a sequence of eye images of two eyes of a user associated with the electronic device; determining a sequence of eye focal positions of the two eyes in the sequence of eye images; and generating a map of convergence angles of the two eyes based on at least the sequence of eye focal positions.

Clause 69. The method of Clause 68, wherein the sequence of visual stimuli corresponds to a plurality of stimulus positions distributed in the 3D virtual environment.

Clause 70. The method of Clause 68 or 69, further comprising: determining a plurality of convergence angles of the two eyes corresponding to the plurality of stimulus positions.

Clause 71. The method of Clause 70, determining the plurality of convergence angles of the two eyes further comprising, for each stimulus position: determining a left eye focal position of a left eye and a right eye focal position of a right eye; determining a gaze point based on the left focal position and the right focal position; and determining a respective convergence angle based on the left eye focal position, the right eye focal position, and the gaze point.

Clause 72. A method of implementing a vision test, comprising: at an electronic device having a head-mounted display (HMD), one or more sensors, one or more processors, and memory: executing a visual assessment application, including displaying a user interface to create a 3D virtual environment; while displaying a sequence of visual hallucination patterns, obtaining a stream of sensor data from the one or more sensors; determining a plurality of user responses to the sequence of visual hallucination patterns based on the stream of sensor data; and determining a type and a severity level of a first visual hallucination condition of a user associated with the electronic device.

Clause 73. The method of Clause 72, further comprising: extracting a plurality of response feature vectors from the plurality of user responses; and applying a hallucination diagnosis model to process the plurality of response feature vectors and generate an output vector.

Clause 74. The method of Clause 73, wherein the output vector includes a plurality of output elements each of which represents a respective severity level of a respective one of a plurality of known hallucination conditions.

Clause 75. The method of Clause 74, further comprising: identifying a first output element greater than a threshold severity; and determining that the first output element corresponds to the type of the first visual hallucination condition.

Clause 76. The method of any of Clauses 73-75, wherein the hallucination diagnosis model includes a classifier neural network, and the output vector includes a plurality of output elements each of which represents a probability of having a respective one of a plurality of known hallucination conditions.

Clause 77. The method of Clause 76, further comprising: identifying a first output element having the greatest value among the plurality of output elements; and determining that the first output element corresponds to the type of the first visual hallucination condition.

Clause 78. The method of Clause 76 or 77, further comprising: identifying two or more output elements that have the greatest values among the plurality of output elements and are greater than a threshold probability, the two or more output elements include a first output element; and determining that the first output element corresponds to the type of the first visual hallucination condition.

Clause 79. The method of Clause 78, further comprising: determining a type and a severity level of each remainder visual hallucination condition distinct from the first visual hallucination condition.

Clause 80. The method of any of Clauses 73-79, wherein each hallucination pattern of the sequence of visual hallucination patterns corresponds to a respective type and a respective severity level of a respective known hallucination condition, and the respective type and the respective severity level of the respective known hallucination condition are processed by the hallucination diagnosis model jointly with the plurality of response feature vectors.

Clause 81. The method of any of Clauses 72-80, wherein the sequence of visual hallucination patterns includes an ordered sequence of known hallucination patterns corresponding to a set of known hallucination conditions, and each known hallucination condition corresponds to a subset of a collection of respective known hallucination patterns.

Clause 82. The method of Clause 81, wherein for each known hallucination condition, the subset of respective known hallucination patterns are arranged according to severity levels of the respective known hallucination condition to the respective known hallucination patterns correspond.

Clause 83. The method of any of Clauses 72-82, wherein the plurality of user response include a user input captured by a subset of one or more first sensors of the electronic device, and the one or more first sensors include a forward facing camera for detecting a hand gesture and a microphone for collecting an audio response.

Clause 84. The method of any of Clauses 72-83, wherein the plurality of user responses includes a spontaneous user response monitored by a subset of one or more second sensors of the electronic device, and the one or more second sensors include one or more of: an eye tracking camera, a heart rate sensor, a body temperature sensor, a blood oxygen level, a Galvanic skin response sensor, a hand gesture camera, a body gesture camera, a microphone, a motion sensor, and a set of one or more brain activity electrodes.

Clause 85. The method of any of Clauses 72-84, wherein the stream of sensor data includes a stream of image data captured by an eye-tracking camera, each respective visual hallucination pattern corresponding to a subset of image data indicating a user's spontaneous response to the respective visual hallucination pattern.

Clause 86. The method of Clause 85, further comprising: extracting eye positions, pupil dilation information, and retinal responses from the stream of image data; and determining a focus level of the user associated with the electronic device.

Clause 87. A method of implementing a vision test, comprising: at an electronic device having a head-mounted display (HMD), one or more sensors, one or more processors, and memory: executing a visual assessment application, including displaying a user interface to create a 3D virtual environment; while displaying a sequence of visual hallucination patterns, obtaining a stream of sensor data from the one or more sensors; extracting a plurality of spontaneous response feature vectors from the sensor data; applying a hallucination diagnosis model to process at least the plurality of spontaneous response feature vectors and generate an output vector. determining a type and a severity level of a first visual hallucination condition of a user associated with the electronic device.

Clause 88. A method of implementing a vision test, comprising: at an electronic device having a head-mounted display (HMD), one or more sensors, one or more processors, and memory: displaying a plurality of visual stimuli concurrently in a 3D virtual environment, each visual stimulus being displayed at a position in the 3D virtual environment according to a display scheme; obtaining a stream of sensor data measured by the one or more sensors; determining a plurality of sequential user responses to the plurality of visual stimuli based on the stream of sensor data; and based on the plurality of sequential user responses, determining an attention indicator indicating an attention capability of the user associated with the electronic device to different visual stimuli.

Clause 89. The method of Clause 88, wherein for each of a first subset of visual stimuli, in accordance with the respective display scheme, the respective visual stimulus is displayed with a respective flickering frequency and an active duty cycle and configured to fade away and emerge at a varying rate.

Clause 90. The method of Clause 89, for each of a second subset of visual stimuli, in accordance with the respective display scheme, the respective visual stimulus is continuously displayed without flickering.

Clause 91. The method of any of Clauses 88-90, wherein each of the plurality of visual stimuli is surrounded by a local context, and the local context is displayed with a context scheme configured to create a respective level of distraction to the respective visual stimuli.

Clause 92. The method of any of Clauses 88-91, wherein each visual stimulus is displayed with a set of display parameters including a display size, a resolution, a contrast level, and a brightness level.

Clause 93. The method of any of Clauses 88-92, further comprising generating an instruction to guide the user to identify the plurality of visual stimuli according to a sequential order.

Clause 94. The method of any of Clauses 88-93, wherein each of the plurality of sequential user responses includes a respective temporal variation of: a left eye position, a right eye position, a gaze point, a pupil size, a saccadic movement level, and a head orientation.

Clause 95. The method of any of Clauses 88-94, wherein the plurality of visual stimuli includes a sequential order of visual stimuli, and each of the plurality of sequential user responses corresponds to a respective sensor, and includes an ordered sequence of user response segments each of which corresponding to a respective visual stimulus.

Clause 96. The method of Clause 95, further comprising for each sequential user response, extracting a plurality of user response feature vectors from the ordered sequence of user response segments.

Clause 97. The method of Clause 96, wherein each user response segment of a first sequential response includes a respective number of sensor data samples, the method further comprising, for a subset set of user response segments of the first sequential response: converting the respective number of sensor data samples to a predefined number of sensor data samples, wherein the predefined number of sensor data samples are processed to extract a respective user response feature vector from the respective user response segment.

Clause 98. The method of any of Clauses 88-95, determining the attention indicator further comprising: applying an attention tracking model to process the plurality of sequential user responses and generate an output vector corresponding to the attention indicator.

Clause 99. The method of Clause 98, wherein the attention tracking model includes a feature extraction model and a feature tracking model, and applying the attention tracking model further comprises: for each sequential user response, extracting a plurality of user response feature vectors corresponding to the respective sequential user response; for each visual stimulus, generating a stimulus feature vector based on characteristics of the respective visual stimulus and an associated local context; and organizing the plurality of user response feature vectors of the plurality of sequential user responses and the stimulus feature vectors of the plurality of visual stimuli in a predefined data structure, forming an input feature structure; and applying the feature tracking model to process the input feature structure and generate the output vector.

Clause 100. The method of Clause 98 or 99, wherein the output vector includes a plurality of output elements, and each output element corresponds to an attention related performance level selected from: a fixation duration, a display size limit, a flickering rate limit, and a distraction susceptibility level.

Clause 101. The method of any of Clauses 88-100, further comprising executing a visual assessment application, including displaying a user interface to create a 3D virtual environment.

Clause 102. A method of implementing a vision test, comprising: at an electronic device having a head-mounted display (HMD), an infrared eye tracking camera, one or more processors, and memory: while displaying a plurality of visual stimuli concurrently in a 3D virtual environment, obtaining infrared video data recorded by the infrared eye tracking camera; determining a plurality of sequential user responses to the plurality of visual stimuli based on the infrared video data; and determining a severity level and a type of an attention deficiency condition for the user associated with the electronic device based on the plurality of sequential user responses.

Clause 103. The method of Clause 102, further comprising executing a visual assessment application, including displaying a user interface to create the 3D virtual environment.

Clause 104. The method of Clause 102 or 103, further comprising: applying an attention tracking model to process the plurality of sequential user responses and generate an output vector including a plurality of output elements, the plurality of output elements including the severity level and the type of the attention deficiency condition and further including one or more of: a deviation from a normal attention profile and a recommendation for further consultation.

Clause 105. A method of implementing a vision test, comprising: at an electronic device having a head-mounted display (HMD), one or more motion sensors, one or more processors, and memory: displaying a destination and a target path leading to the destination in a 3D virtual environment, the target path following at least one direction; rendering a request for a user associated with the electronic device to follow the target path to reach the destination; obtaining a stream of sensor data from the one or more motion sensors, the stream of sensor data being collected from the one or more motion sensors while the user moves along the target path; and based on the stream of sensor data, determining a directionality indicator of the user's visual system quantitatively representing a capability of the user's visual system following the at least one direction.

Clause 106. The method of Clause 105, wherein the one or more motion sensors of the electronic device includes an outward camera, a set of accelerometers, and a set of a gyroscopes.

Clause 107. The method of Clause 105 or 106, wherein the electronic device further includes one or two controllers configured to be held by the user's hands, and the one or more motion sensors further includes supplemental sensors located in the one or two controllers.

Clause 108. The method of any of Clauses 105-107, further comprising: reconstructing a user path based on the stream of sensor data; and comparing the user path with the target path to determine a path fitting level, wherein the directionality indicator is determined based on the path fitting level.

Clause 109. The method of Clause 108, wherein the user path includes a plurality of positions, further comprising: determining a speed of the user associated with each of the plurality of positions based on the stream of sensor data, wherein the directionality indicator is determined based on the path fitting level and the speeds of the user at the plurality of positions.

Clause 110. The method of any of Clauses 105-109, wherein the directionality indicator of the user's visual system is generated after the user reaches the destination.

Clause 111. The method of Clause 110, determining the directionality indicator of the user's visual system further comprising: extracting a sensor feature vector based on a subset of sensor data corresponding to each of the one or more motion sensors; selecting a directionality analysis model from a plurality of predefined model options based on the destination and the target path; and applying the selected directionality analysis model to process the sensor feature vectors of the one or more motion sensors and generate an output vector, wherein the directionality indicator is determined based on the output vector.

Clause 112. The method of Clause 111, wherein the output vector includes a subset of respective elements corresponding to each of a plurality of directions, and the directionality indicator includes a plurality of directionality scores corresponding to the plurality of directions, determining the directionality indicator of the user's visual system further comprising: generating the plurality of directionality scores based on the respective elements of the output vector corresponding to the plurality of directions.

Clause 113. The method of any of Clauses 105-112, wherein the directionality indicator of the user's visual system is generated concurrently while the user moves along the target path.

Clause 114. The method of Clause 113, wherein each sensor has a sensor sampling frequency, and the directionality indicator is generated at a directionality assessment frequency corresponding to a directionality time window, the directionality assessment frequency is smaller than the sensor sampling frequency.

Clause 115. The method of Clause 114, further comprising: after determining a set of first directionality indicator samples, dynamically adjusting at least the target path associated with the destination for one or more subsequent directionality time windows.

Clause 116. The method of Clause 115, wherein dynamically adjusting at least the target path further comprises: determining whether the set of first directionality indicator samples satisfies a directionality criterion; and adjusting a difficulty level of the target path for the one or more subsequent directionality time windows.

Clause 117. The method of any of Clauses 105-116, further comprising executing a visual assessment application, including displaying a user interface to create the 3D virtual environment.

Clause 118. The method of Clause 117, further comprising: executing a sport training application configured to manage training of athletes, wherein the visual assessment application is coupled to the sport training application via an application programming interface (API), and the sport training application is executed within the visual assessment application via the API; and feeding the directionality indicator of the user's visual system to the sport training application via the API.

Clause 119. A method of implementing a vision test, comprising: at an electronic device having a head-mounted display (HMD), one or more motion sensors, one or more processors, and memory: executing a sport training application for athlete training; displaying a destination and a target path leading to the destination in a 3D virtual environment; obtaining a stream of sensor data from the one or more motion sensors, the stream of sensor data being collected while the user moves along the target path; and based on the stream of sensor data, determining a directionality indicator of the user's visual system quantitatively representing a direction managing capability of the user's visual system.

Clause 120. The method of Clause 119, wherein the sport training application is applied to train basketball players.

Clause 121. The method of Clause 119 or 120, wherein the directionality indicator includes a plurality of first scores each of which corresponds to a respective one of a plurality of directions.

Clause 122. The method of Clause 121, wherein the directionality indicator includes a plurality of second scores each of which corresponds to a respective speed to respond to a change between respective two of a plurality of directions.

Clause 123. An interactive virtual-reality method for performing a virtual vision test and displaying media, as discussed in any of Clauses 1-122.

Clause 124. A non-transitory computer readable storage medium, storing one or more programs for execution by one or more processors of a computer system, the one or more programs including instructions for implementing a method in any of Clauses 1-122.

Clause 125. A computer system, comprising: one or more processors; and memory for storing one or more programs for execution by the one or more processors, the one or more programs including instructions for implementing a method in any of Clauses 1-122.

In some embodiments, any of the above clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

### Further Considerations

As used herein, the word module refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase at least one of preceding a series of items, with the term and or or to separate any of the items, modifies the list as a whole, rather than each member of the list (i.e., each item). The phrase at least one of does not require selection of at least one of each item listed; rather, the phrase allows a meaning that includes at least one of any one of the items, and/or at least one of any combination of the items, and/or at least one of each of the items. By way of example, the phrases at least one of A, B, and C or at least one of A, B, or C each refer to only A, only B, or only C; any combination of A, B, and C; and/or at least one of each of A, B, and C.

Terms such as top, bottom, front, rear and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term include, have, or the like is used in the description or the claims, such term is intended to be inclusive in a manner similar to the term comprise as comprise is interpreted when employed as a transitional word in a claim.

As used herein, the term about is relative to the actual value stated, as will be appreciated by those of skill in the art, and allows for approximations, inaccuracies and limits of measurement under the relevant circumstances. In one or more aspects, the terms about, substantially, and approximately may provide an industry-accepted tolerance for their corresponding terms and/or relativity between items.

As used herein, the term comprising indicates the presence of the specified integer(s), but allows for the possibility of other integers, unspecified. This term does not imply any particular proportion of the specified integers. Variations of the word comprising, such as comprise and comprises, have correspondingly similar meanings.

The word exemplary is used herein to mean serving as an example, instance, or illustration. Any embodiment described herein as exemplary is not necessarily to be construed as preferred or advantageous over other embodiments.

A reference to an element in the singular is not intended to mean one and only one unless specifically stated, but rather one or more. Pronouns in the masculine (e.g., his) include the feminine and neuter gender (e.g., her and its) and vice versa. The term some refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope. In addition, it is not necessary for a device or method to address every problem that is solvable (or possess every advantage that is achievable) by different embodiments of the disclosure in order to be encompassed within the scope of the disclosure. The use herein of can and derivatives thereof shall be understood in the sense of possibly or optionally as opposed to an affirmative capability.

What is claimed is:

1. A method for implementing a vision test, comprising:
at an electronic device having a head-mounted display (HMD), one or more processors, and memory:
executing a visual assessment application, including displaying a user interface to create a 3D virtual environment;
while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment;
determining a sequence of 3D gaze positions of the eyes in the 3D virtual environment based on the sequence of eye images; and
determining a visual processing performance factor for the user based on the sequence of stimulus positions and the sequence of 3D gaze positions.

2. The method of claim 1, further comprising:
adaptively determining one or more display parameters for displaying media content on the HMD based on the visual processing performance factor.

3. The method of claim 1, determining the sequence of 3D gaze positions of the eyes further comprising:
extracting a region of interest (ROI) image of the two eyes from each eye image to form a sequence of ROI images based on the sequence of eye images; and
applying a focus tracking model to process the sequence of ROI images and generate an output vector including the sequence of 3D gaze positions.

4. The method of claim 1, determining the sequence of 3D gaze positions of the eyes further comprising, for each eye image captured at a respective time:
generating a region of interest (ROI) image of the two eyes in the respective eye image;
determining a respective 3D gaze position based on the ROI image.

5. The method of claim 1, determining the sequence of 3D gaze positions of the eyes further comprising, for each eye image captured at a respective time:
identifying a left eye center and a right eye center;
determining a left line of sight and a right line of sight; and
determining a respective 3D gaze position as an intersection point of the left line of sight and the right line of sight.

6. The method of claim 1, wherein determining the visual processing performance factor further comprising:
determining at least one of a visual processing speed and a visual processing accuracy.

7. The method of claim 1, further comprising:
based on the visual processing performance factor, determining a visual processing deficiency condition.

8. The method of claim 1, wherein each pair of two successive visual stimuli of the sequence of visual stimuli has a respective stimulus position change, determining the visual processing performance factor further comprising:
determining a relationship of the visual processing performance factor and the respective stimulus position change.

9. The method of claim 1, determining the visual processing performance factor further comprising:
for each stimulus position, identifying a respective set of one or more 3D gaze positions that satisfy a predefined response criterion.

10. The method of claim 9, wherein the predefined response criterion requires that for each stimulus position, the respective set of one or more 3D gaze positions are located within a respective physical range surrounding the respective stimulus position.

11. The method of claim 9, wherein each stimulus position corresponds to a stimulus time, and each eye focal position corresponds to a focal time, wherein the visual processing performance factor is determined based on the stimulus time of each of a subset of stimuli and the focal time of each of the respective set of one or more 3D gaze positions corresponding to each stimulus position.

12. The method of claim 11, determining the visual processing performance factor further comprising:
for each stimulus position, identifying a respective response time corresponding to a first focal point having the earliest focal time among the respective set of one or more 3D gaze positions; and
determining a visual processing speed based on one or more respective response times of a subset of one or more stimulus positions.

13. The method of claim 11, determining the visual processing performance factor further comprising;
for each stimulus position, determining an average eye focal position of the respective set of one or more 3D gaze positions, and determining a position offset based on the average eye focal position and the stimulus position; and
determining a visual processing accuracy based on the position offsets of the stimulus positions.

14. The method of claim 1, further comprising:
determining a sequence of pupil sizes of at least one eye based on the sequence of eye images;
determining a focus level of the sequence of pupil size; and
adjusting the visual processing performance factor based on the focus level.

15. The method of claim 1, wherein the sequence of visual stimuli includes a known stimulus that is displayed sequentially at the sequence of stimulus positions.

16. The method of claim 1, further comprising:
applying a visual processing assessment model to receive at least the sequence of eye images and the sequence of stimulus positions as inputs and generate the visual processing performance factor for the user.

17. The method of claim 16, wherein the sequence of eye images correspond to a sequence of ROI images of eye regions, obtaining the sequence of eye images further comprising extracting each eye image from tracking images captured by an eye tracking camera.

18. The method of claim 16, applying the visual processing assessment model further comprising:
extracting an image feature from each eye image; and
generating a model input feature by arranging the image features of the sequence of eye images and the sequence of stimulus positions according to a predefined input data structure, wherein the model input feature is fed into the visual processing assessment model.

19. A non-transitory computer readable storage medium, storing one or more programs for execution by one or more processors of an electronic device having a HMD, the one or more programs including instructions for:
executing a visual assessment application, including displaying a user interface to create a 3D virtual environment;
while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment;
determining a sequence of 3D gaze positions of the eyes in the 3D virtual environment based on the sequence of eye images; and
determining a visual processing performance factor for the user based on the sequence of stimulus positions and the sequence of 3D gaze positions.

20. A computer system, comprising:
an HMD;
one or more processors; and
memory for storing one or more programs for execution by the one or more processors, the one or more programs including instructions for:
executing a visual assessment application, including displaying a user interface to create a 3D virtual environment;
while displaying a sequence of visual stimuli on the user interface, obtaining a sequence of eye images of two eyes of a user associated with the electronic device, wherein the sequence of visual stimuli corresponds to a sequence of stimulus positions in the 3D virtual environment;
determining a sequence of 3D gaze positions of the eyes in the 3D virtual environment based on the sequence of eye images; and
determining a visual processing performance factor for the user based on the sequence of stimulus positions and the sequence of 3D gaze positions.
